SEQUENCE LISTING (41 sequences)

SEQ ID NO: 1 - DNA, 54 bp, synthetic construct
  source 1..54: mol_type = other DNA
gattcatcagattcttctgattcatccgactcttctgacagttcagacagctct

SEQ ID NO: 2 - DNA, 42 bp, synthetic construct
  source 1..42: mol_type = other DNA
gatgatgatgatgatgatgatgatgatgatgatgatgatgat

SEQ ID NO: 3 - DNA, 327 bp, synthetic construct
  source 1..327: mol_type = other DNA
tttgtcttttatttcaggtcccagatctagggctctgcgtttgctccaggtagtccgctgctcccttgggcctgggcccactgacagccctggtgcctctggccggctgcacacctcctggcgggcagctgtgttactgtaggatcgagagggatgttctggcaatacctgtccctctccttactacagtaacacggaggcctgccctgactgcccacggtgccgtggccaaagaggatctaagggcaccgctgagggcctacctaaccatcgtggggaataaggacagtgtcacccctgcaggggatccggtggtggtgcaaatca

SEQ ID NO: 4 - DNA, 279 bp, synthetic construct
  source 1..279: mol_type = other DNA
gatctggcagccttggagtgggttcctgccccctcgggcacacaaacagagctgaagaccaccctgggcacctccttggctggccgcatacctcctggcgggcagctgtgtacaaactacttgagagcaggtgttctggtggtacccacctgctctgtaatagtttgtacacagaggcctgcctggccctcgagagactgccctgactgaaggccctatcaggtgggggaggggatcctgatagagggcactgctgccactgttggggcccaagctgca

SEQ ID NO: 5 - DNA, 327 bp, synthetic construct
  source 1..327: mol_type = other DNA
tttgtcttttatttcaggtcccagatctagggctctgcgtttgctccaggtagtccgctgctcccttgggcctgggcccactgacagccctggtgcctctggccggctgcacacctcctggcgggcagctgtgtacaaactacttgagagcaggtgttctggcaatacctgcctgctctgtaatagtttgtacacggaggcctgccctgactgcccacggtgccgtggccaaagaggatctaagggcaccgctgagggcctacctaaccatcgtggggaataaggacagtgtcacccctgcaggggatccggtggtggtgcaaatca

SEQ ID NO: 6 - DNA, 325 bp, synthetic construct
  source 1..325: mol_type = other DNA
gtcttttatttcaggtcccagatcttagggctctgcgtttgctccaggtagtccgctgctcccttgggcctgggcccactgacagccctggtgcctctggccggctgcacacctcctggcgggcagctgtgtttccatggtaagttcaaggctgttctggcaatacctggccttgaagatgccatggaaacacggaggcctgccctgactgcccacggtgccgtggccaaagaggatctaagggcaccgctgagggcctacctaaccatcgtggggaataaggacagtgtcacccccctgcaggggatccggtggtggtgcaaat

SEQ ID NO: 7 - DNA, 279 bp, synthetic construct
  source 1..279: mol_type = other DNA
gatctagggctctgcgtttgctccaggtagtccgctgctcccttgggcctgggcccactgacagccctggtgcctctggccggctgcacacctcctggcgggcagctgtgtgacctctgtggcatcattcctgttctggcaatacctgggaatgatcgcgcagaggtcacacggaggcctgccctgactgcccacggtgccgtggccaaagaggatctaagggcaccgctgagggcctacctaaccatcgtggggaataaggacagtgtcacccctgca

SEQ ID NO: 8 - DNA, 693 bp, synthetic construct
  source 1..693: mol_type = other DNA
gatctagggctctgcgtttgctccaggtagtccgctgctcccttgggcctgggcccactgacagccctggtgcctctggccggctgcacacctcctggcgggcagctgtgtgacctctgtggcatcattcctgttctggcaatacctgggaatgatcgcgcagaggtcacacggaggcctgccctgactgcccacggtgccgtggccaaagaggatctaagggcaccgctgagggcctacctaaccatcgtggggaataaggacagtgtcacccctgcaggggatccggtggtggtgcaaatcaaagaactgctcctcagtggatgttgcctttacttctaggcctgtacggaagtgttacttctgctctaaaagctgcggaattgtacccgcggccgatccaccggtcgccaccatggggcagccttggagtgggttcctgccccctcgggcacacaaacagagctgaagaccaccctgggcacctccttggctggccgcatacctcctggcgggcagctgtgtacaaactacttgagagcaggtgttctggtggtacccacctgctctgtaatagtttgtacacagaggcctgcctggccctcgagagactgccctgactgaaggccctatcaggtgggggaggggatcctgatagagggcactgctgccactgttggggcccaagaagct

SEQ ID NO: 9 - DNA, 269 bp, synthetic construct
  source 1..269: mol_type = other DNA
ggcagccttggagtgggttcctgccccctcgggcacacaaacagagctgaagaccaccctgggcacctccttggctggccgcatacctcctggcgggcagctgtgtttccatggtaagttcaaggctgttctggtggtacccagccttgaagatgccatggaaacacagaggcctgcctggccctcgagagactgccctgactgaaggccctatcaggtgggggaggggatcctgatagagggcactgctgccactgttggggcccaag

SEQ ID NO: 10 - DNA, 269 bp, synthetic construct
  source 1..269: mol_type = other DNA
ggcagccttggagtgggttcctgccccctcgggcacacaaacagagctgaagaccaccctgggcacctccttggctggccgcatacctcctggcgggcagctgtgtccatggtaagttcaaggctgtgttctggtggtacccacagccttgttcctaccatggacacagaggcctgcctggccctcgagagactgccctgactgaaggccctatcaggtgggggaggggatcctgatagagggcactgctgccactgttggggcccaag

SEQ ID NO: 11 - protein, 18 aa, synthetic construct
DSSDSSDSSDSSDSSDSS

SEQ ID NO: 12 - protein, 19 aa, synthetic construct
  MOD_RES 2: note = is a gamma-carboxylated glutamic acid
  MOD_RES 6: note = is a gamma-carboxylated glutamic acid
  MOD_RES 9: note = is a gamma-carboxylated glutamic acid
  MOD_RES 11: note = is a gamma-carboxylated glutamic acid
  MOD_RES 15: note = is a gamma-carboxylated glutamic acid
  MOD_RES 18: note = is a gamma-carboxylated glutamic acid
CEPRREVAELEPRREVAEL

SEQ ID NO: 13 - protein, 5 aa, synthetic construct
GGGGS

SEQ ID NO: 14 - protein, 25 aa, synthetic construct
  VARIANT 3..25: note = may be absent
GGGGGGGGGGGGGGGGGGGGGGGGG

SEQ ID NO: 15 - protein, 26 aa, synthetic construct
  VARIANT 3..25: note = may be absent
GGGGGGGGGGGGGGGGGGGGGGGGGS

SEQ ID NO: 16 - protein, 50 aa, synthetic construct
  VARIANT 5..50: note = may be absent
GSGSGSGSGSGSGSGSGSGSGSGSGSGSGSGSGSGSGSGSGSGSGSGSGS

SEQ ID NO: 17 - protein, 100 aa, synthetic construct
  VARIANT 9..100: note = may be absent
GGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSG

SEQ ID NO: 18 - DNA, 57 bp, synthetic construct
  source 1..57: mol_type = other DNA
ccgggccttgaacttaccatggaaactcgagtttccatggtaagttcaaggcttttt

SEQ ID NO: 19 - DNA, 22 bp, synthetic construct
  source 1..22: mol_type = other DNA
gccctatgtgtgcaagcactgt

SEQ ID NO: 20 - DNA, 21 bp, synthetic construct
  source 1..21: mol_type = other DNA
agaggccattcagacgagtgt

SEQ ID NO: 21 - DNA, 20 bp, synthetic construct
  source 1..20: mol_type = other DNA
actgagcaagagaggcccta

SEQ ID NO: 22 - DNA, 20 bp, synthetic construct
  source 1..20: mol_type = other DNA
agggaaatcgtgcgtgacat

SEQ ID NO: 23 - DNA, 24 bp, synthetic construct
  source 1..24: mol_type = other DNA
ccaagcaaggcatgtccagacacc

SEQ ID NO: 24 - DNA, 22 bp, synthetic construct
  source 1..22: mol_type = other DNA
gcagatgaacctgaaggatacc

SEQ ID NO: 25 - DNA, 24 bp, synthetic construct
  source 1..24: mol_type = other DNA
cttcaggaatgatgccacagaggt

SEQ ID NO: 26 - DNA, 23 bp, synthetic construct
  source 1..23: mol_type = other DNA
ctggtgaaaaggacctctcgaag

SEQ ID NO: 27 - DNA, 20 bp, synthetic construct
  source 1..20: mol_type = other DNA
agcaaagaccccaacgagaa

SEQ ID NO: 28 - DNA, 22 bp, synthetic construct
  source 1..22: mol_type = other DNA
agtcctggaacaggtcgtcact

SEQ ID NO: 29 - DNA, 20 bp, synthetic construct
  source 1..20: mol_type = other DNA
ctgcggaagctgagagatgt

SEQ ID NO: 30 - DNA, 20 bp, synthetic construct
  source 1..20: mol_type = other DNA
tatgggggtctgggatggaa

SEQ ID NO: 31 - DNA, 20 bp, synthetic construct
  source 1..20: mol_type = other DNA
gaaccgctcgttgccaatag

SEQ ID NO: 32 - DNA, 24 bp, synthetic construct
  source 1..24: mol_type = other DNA
gcctgacagtgaggatgggtaggg

SEQ ID NO: 33 - DNA, 22 bp, synthetic construct
  source 1..22: mol_type = other DNA
ttgatgccatctcgtatgtagg

SEQ ID NO: 34 - DNA, 24 bp, synthetic construct
  source 1..24: mol_type = other DNA
atctttggcgtcatagggatggtg

SEQ ID NO: 35 - DNA, 24 bp, synthetic construct
  source 1..24: mol_type = other DNA
ccagtttcactaatgacacaaacg

SEQ ID NO: 36 - DNA, 15 bp, synthetic construct
  source 1..15: mol_type = other DNA
ggcggcggtcacgaa

SEQ ID NO: 37 - DNA, 22 bp, synthetic construct
  source 1..22: mol_type = other DNA
cgcgatcacatggtcctgctgg

SEQ ID NO: 38 - RNA, 69 nt, synthetic construct
  source 1..69: mol_type = other RNA
ctgtggtgcattgtagttgcattgcatgttctggcaatacctgtgcaatgtttccacagtgcatcacgg

SEQ ID NO: 39 - RNA, 69 nt, synthetic construct
  source 1..69: mol_type = other RNA
ctgtggtgcattgtagttgcattgcatgttctggtggtacccatgcaatgtttccacagtgcatcacag

SEQ ID NO: 40 - protein, 6 aa, synthetic construct
RGDCYK

SEQ ID NO: 41 - protein, 8 aa, synthetic construct
  SITE 1..8: note = D-Asp
DDDDDDDD

(19) United States
(12) Patent Application Publication
(10) Pub. No.: US 2025/0388925 A1
(43) Pub. Date: Dec. 25, 2025

(54) WNT-MODULATING GENE SILENCERS AS BONE ANABOLIC THERAPY FOR OSTEOPOROSIS AND CRITICAL-SIZED BONE DEFECT

(71) Applicant: University of Massachusetts, Boston, MA (US)

(72) Inventors: Jae-Hyuck Shim, Worcester, MA (US); Guangping Gao, Worcester, MA (US)

(73) Assignee: University of Massachusetts, Boston, MA (US)

(21) Appl. No.: 18/707,207

(22) PCT Filed: Nov. 3, 2022
(86) PCT No.: PCT/US2022/079238

§ 371 Date: May 3, 2024

Related U.S. Application Data
(60) Provisional application No. 63/275,888, filed on Nov. 4, 2021.

(51) Int. Cl. C12N 15/86; A61K 33/06; A61L 27/36; A61L 27/54; C07K 14/005; C12N 15/113

(52) U.S. Cl. CPC C12N 15/86; A61K 33/06; A61L 27/365; A61L 27/54; C07K 14/005; C12N 15/113; A61L 2300/258; A61L 2300/432; A61L 2430/02; C12N 2310/141; C12N 2750/14122; C12N 2750/14143

(57) ABSTRACT

Aspects of the disclosure relate to compositions and methods for treating diseases or disorders associated with bone fracture and critical-sized bone defect. In some embodiments, the disclosure provides isolated nucleic acids and expression constructs (e.g., rAAVs, etc.) that encode one or both of the inhibitory nucleic acids targeting Schnurri 3 (SHN3) and the inhibitory nucleic acids targeting sclerostin (SOST).

### RELATED APPLICATIONS

This application is a national stage filing under 35 U.S.C. § 371 of international PCT application PCT/US2022/079238, filed Nov. 3, 2022, which claims priority under 35 U.S.C. § 119(e) to U.S. provisional patent application, U.S. Ser. No. 63/275,888, filed Nov. 4, 2021, the entire contents of each of which are incorporated by reference herein.

### REFERENCE TO AN ELECTRONIC SEQUENCE LISTING

The contents of the electronic sequence listing (U012070167US01-SUBSEQ-KZM.xml; Size: 44,305 bytes; and Date of Creation: Apr. 30, 2024) is herein incorporated by reference in its entirety.

### BACKGROUND

Osteoporosis is a disease characterized by loss of bone mass and is a major source of frailty and suffering associated with aging. An estimated 10 million Americans over age 50 have osteoporosis, and osteoporosis-related fractures occur in approximately 1.5 million individuals per year, with serious health consequences. Most existing therapeutic agents for osteoporosis inhibit resorption of bone by osteoclasts (OCs) and this inhibition is accompanied by numerous side effects, including atypical fractures and osteonecrosis of the jaw.

### SUMMARY OF INVENTION

Aspects of the disclosure relate to compositions and methods for treating osteoporosis and critical-sized bone defect in a subject. The disclosure is based, in part, on isolated nucleic acids and expression constructs encoding at least one transgene, such as inhibitory nucleic acids or proteins, that can prevent bone formation, reverse bone loss, and promote fracture union, while limiting side effects in bone tissues and non-target tissues.

Accordingly, in some aspects, the disclosure provides a bone graft substitute comprising a recombinant adeno-associated virus (rAAV) and hydroxyapatite (HA) attached to the bone graft substitute. In some embodiments, the rAAV comprises a capsid protein comprising a peptide motif and an isolated nucleic acid comprising a nucleic acid sequence encoding an inhibitory nucleic acid targeting sclerostin (SOST), schnurri-3 (SHN3), or SOST and SHN3. In some embodiments, the bone graft substitute is for the implantation to a subject.

In some embodiments, the bone graft substitute is an allogeneic bone graft. In some embodiments, the capsid protein is an AAV9 capsid protein.

In some embodiments, the bone graft substitute is incubated ex vivo with the rAAV prior to implantation to the subject. In some embodiments, the bone graft substitute is incubated ex vivo with human bone marrow-derived stromal cells prior to implantation to the subject. In some embodiments, the bone graft substitute is incubated ex vivo with a composition comprising cells of osteoblastic lineage prior to implantation to the subject.

In some aspects, the disclosure provides an isolated nucleic acid comprising a transgene comprising a chicken β-actin (CB) promoter operably linked to a nucleic acid sequence encoding an inhibitory nucleic acid targeting sclerostin (SOST), schnurri-3 (SHN3), or SOST and SHN3.

In some embodiments, the transgene encodes an inhibitory nucleic acid selected from the group consisting of dsRNA, siRNA, shRNA, miRNA, and artificial miRNA (amiRNA). In some embodiments, the inhibitory nucleic acid is an ami-RNA comprising a human miRNA backbone. In some embodiments, the inhibitory nucleic acid is an ami-RNA comprising a mouse miRNA backbone. In some embodiments, the mouse miRNA backbone is a mouse miR-33 backbone.

In some embodiments, the inhibitory nucleic acid targets SHN3. In some embodiments, the inhibitory nucleic acid is encoded by a nucleic acid comprising the sequence set forth in any one of SEQ ID NOs: 4, 5, 6, 9 and 10.

In some embodiments, the inhibitory nucleic acid targets SOST. In some embodiments, the inhibitory nucleic acid is encoded by a nucleic acid comprising the sequence set forth in SEQ ID NO: 7.

In some embodiments, the inhibitory nucleic acids target SHN3 and SOST. In some embodiments, the inhibitory nucleic acid is encoded by a nucleic acid comprising the sequence set forth in SEQ ID NO: 8.

In some embodiments, the transgene further comprises a CMV enhancer sequence.

In some embodiments, the transgene is flanked by adeno-associated virus (AAV) inverted terminal repeats (ITRs). In some embodiments, the AAV ITRs are AAV2 ITRs.

In some embodiments, the present disclosure provides an isolated nucleic acid comprising or encoding a sequence set forth in any one of SEQ ID NOs: 1-37.

In some embodiments, the present disclosure provides a vector comprising an isolated nucleic acid as described herein. In some embodiments, the vector is a plasmid, bacmid, cosmid, viral, closed-ended linear DNA (ceDNA), or Baculovirus vector. In some embodiments, the vector is a recombinant adeno-associated virus (rAAV) vector, retroviral vector, or adenoviral vector.

In some aspects, the present disclosure provides a recombinant adeno-associated virus (rAAV) comprising: (i) an isolated nucleic acid as described herein; and (ii) at least one AAV capsid protein. In some embodiments, the AAV capsid protein is of a serotype selected from AAV1, AAV2, AAV3, AAV4, AAV5, AAV6, AAV6.2, AAV7, AAV8, AAV9, AAV.rh8, AAV.rh10, AAV.rh39, AAV.rh43, AAV2/2-66, AAV2/2-84, and AAV2/2-125, or a variant of any of the foregoing.

In some embodiments, the AAV capsid protein comprises the amino acid sequence DSSDSSDSSDSSDSSDSS (SEQ ID NO: 11).

In some embodiments, the rAAV comprises an attachment to a hydroxyapatite (HA) scaffold. In some embodiments, the attachment to the HA scaffold improves the bone-specific tropism of the rAAV. In some embodiments, the rAAV is rAAV9.

In some embodiments, the present disclosure provides a composition comprising the rAAV and a pharmaceutically acceptable excipient.

In some embodiments, the present disclosure provides a bone graft substitute comprising the composition as described herein. In some embodiments, the bone graft substitute is selected from an autologous bone graft, an allogeneic bone graft, a decellularized bone matrix, a hydroxyapatite scaffold, a calcium-phosphate scaffold, and a skeletal bone organoid obtained from mesenchymal stem cells (MSCs). In some embodiments, the bone graft substitute is incubated ex vivo, prior to implantation, with a composition comprising cells of osteoblastic lineage, an rAAV vector as disclosed herein, or a cell comprising the rAAV vector as disclosed herein.

In some aspects, the disclosure provides a method for delivering a transgene to a bone tissue in a subject, the method comprises administering to the subject an isolated nucleic acid, vector, rAAV, or bone graft substitute as described herein.

In some aspects, the disclosure provides a method for treating a disease or disorder associated with bone fracture and critical-sized bone defect in a subject, the method comprising administering to the subject an isolated nucleic acid, vector, rAAV, or bone graft substitute as described herein.

In some aspects, the disclosure provides a method for treating a disease or disorder associated with osteoporosis in a subject, the method comprising administering to the subject an isolated nucleic acid, vector, rAAV, or bone graft substitute as described herein.

In some aspects, the disclosure provides a method for improving bone formation and/or bone healing in a subject, the method comprising administering to the subject an isolated nucleic acid, vector, rAAV, or bone graft substitute as described herein.

In some aspects, the disclosure provides a method for stimulating bone regeneration and/or reversing bone loss in a subject, the method comprising administering to the subject an isolated nucleic acid, vector, rAAV, or bone graft substitute as described herein.

In some embodiments, the administration occurs by injection. In some embodiments, the injection is systemic injection or local injection. In some embodiments, the systemic injection comprises intravenous injection. In some embodiments, the local injection comprises intramuscular (IM) injection, knee injection, or femoral intramedullary injection.

In some embodiments, the administration results in an increase of receptor activator of nuclear factor kappa-B ligand (RANKL), osteoprotegerin (OPG), Axin2 and/or Lef1.

In some embodiments, the subject is a human.

### BRIEF DESCRIPTION OF DRAWINGS

FIGS. 1A-1S show generation of bone-targeting AAVs carrying WNT-modulating gene silencers. FIG. 1A shows mRNA levels of Shn3 and Sost in in the tibial bone RNA of 2- and 22-month-old mice were measured by qPCR analysis and normalized to Hprt (n=5/group). FIGS. 1B and 1C show pictures and X-ray images showing the kinetics of tibial fracture healing (FIG. 1B) and tissue RNA harvested from the injured sites were subjected to measure mRNA levels of Shn3 and Sost (FIG. 1C, n=5/group). FIGS. 1D-1F show mRNA levels of Shn3 and Sost in the tibias treated with rAAV9 carrying amiR-ctrl, amiR-shn3 (FIG. 1D) or amiR-sost 2 (FIG. 1F) or in the tibias of 5-month-old Shn3^{PrxI} mice (FIG. 1E, n=5-8/group). FIG. 1G shows diagram of the AAV vector genome containing a CMV enhancer/chicken β-actin promoter (CBA), amiR-sost, hs-amiR-shn3, or amiR-Sost/hs-amiR-shn3, an Egfp reporter gene (EGFP), β-globin polyA sequence (polyA), and terminal repeat (TR). FIG. 1H shows Nucleotide sequences of mouse and human miR-33 that include mature and seed sequences of miR-33 (SEQ ID NOs: 38-39). FIGS. 11 and J show a single dose (5×10¹³ vg/kg) of rAAV9.egfp carrying amiR-ctrl, amiR-shn3, or hs-amiR-shn3 was i.v. injected into 2-month-old mice and two months later, mRNA levels of shn3 in the tibias were measured by qPCR analysis and normalized to Bact (FIG. 1I). MicroCT analysis showing femoral bone mass in AAV-treated mice. Relative quantification (FIG. 1J, left) and representative 3D-reconstruction (FIG. 1J, right) are displayed. Trabecular bone volume/total volume (Tb. BV/TV) were measured. Scale bar, 500 μm (f). FIG. 1K shows Ocy454 osteocytic cell line was incubated with AAV vectors, cultured for 6 days, and immunoblotted with the indicated antibodies. FIGS. 1L and 1M show AAV-treated Ocy454 cells were transfected with β-catenin-responsive reporter gene (Top-flash Luc), cultured for 6 days in the presence of rWNT3a, and luciferase activity was measured (FIG. 1L). Alternatively, mRNA levels of β-catenin target genes, Axin2 and Lef1, were assessed by qPCR analysis (FIG. 1M, n=5/group). FIG. 10 shows Flag-SHN3-expressing plasmid was transfected into HEK293 cells along with vector control or a plasmid encoding hs-amiR-shn3 or amiR-ctrl and two days later, cell lysates were immunoblotted with anti-Flag antibody. Hsp90 was used as a loading control. FIG. 1P shows Ocy454 osteocytic cell line was incubated with rAAV9 carrying amiR-ctrl, amiR-shn3, amiR-sost, or amiR-shn3/sost, cultured under differentiation conditions for 6 days, and Shn3 and Sost mRNA levels were measured by RT-PCR analysis. Values represent mean±SD by an unpaired two-tailed Student's t-test and one-way ANOVA test. ns, not significant. Values represent mean±SD by an unpaired two-tailed Student's t-test (FIGS. 1A, 1C, and 1D-F) and one-way ANOVA test (FIGS. 11, 1J, 1L, and 1M). ns: not significant. FIG. 1N shows 1-month-old TCF/LEF1-GFP reporter mice were i.v. injected with a single dose of rAAV9.mCherry (5×10¹³ vg/kg) and two weeks later, expression of GFP and/or mCherry in the cryosectioned femurs was monitored by fluorescence microscopy (n=3/group). FIGS. 1Q-1S show 1-month-old TCF/LEF1-GFP reporter mice (gray box) were i.v. injected with a single dose of rAAV9 carrying amiR-ctrl or amiR-sost/shn3 (5×10¹³ vg/kg) and two weeks later, mRNA levels of Shn3 and Sost (FIG. 1Q) and egfp and Lef1 (FIG. 1R) in the tibia were measured by RT-PCR and normalized to b-actin (n=5/group). Alternatively, protein lysates from the femur were immunoblotted for b-catenin. Gapdh was used as a loading control (FIG. 1S). 1-month-old wildtype mice were used as a negative control. Values represent mean±SD by one-way ANOVA test (FIGS. 1Q, 1R). CB: cortical bone, TB: trabecular bone, BM: bone marrow.

FIGS. 2A-2D show AAV-mediated silencing of Sost expression increases bone mass in mice. FIG. 2A shows diagram of the rAAV9 construct containing a CMV enhancer/chicken β-actin promoter (CB), amiR-ctrl, amiR-Sost1, or amiR-Sost2, an Egfp reporter gene (EGFP), β-globin polyA sequence (polyA), and terminal repeat (TR). FIGS. 2B-2D show a single dose (5×10¹³ vg/kg) of rAAV9.egfp carrying amiR-ctrl, amiR-Sost1, or amiR-Sost2 was i.v. injected into 12-week-old mice and 2 months later, EGFP expression was assessed by fluorescence microscopy of cryo-sectioned femurs (FIG. 2B). Scale bar, 250 μm (FIG. 2B, left) and 25 m (B, right). BM, bone marrow; M, muscle; Cb, cortical bone. Sost mRNA levels in the tibia of AAV-treated mice were assessed by RT-PCR (FIG. 2C). MicroCT analysis showing femoral bone mass in AAV-treated mice. Relative quantification (FIG. 2D, left) and representative 3D-reconstruction (FIG. 2D, right) are displayed. Trabecular bone volume/total volume (Tb. BV/TV), trabecular number (Tb. N), and trabecular thickness (Tb.Th) were measured. Scale bar, 500 μm. Values represent mean±SD by an unpaired two-tailed Student's t-test and one-way ANOVA test. ns, not significant.

FIGS. 3A and 3B show generation of bone-targeting AAVs carrying WNT-modulating dual silencers targeting Shn3 and Sost. FIG. 3A shows HEK293 cells were transiently transfected with the plasmids expressing hs-amiR-shn3 or amiR-ctrl along with Flag-SHN3-expressing plasmid. 2 days later, cells were lysed and immunoblotted with anti-Flag antibody. Immunoblotting for Hsp90 was used as a loading control. FIG. 3B shows Ocy454 osteocytic cell line was incubated with AAV vectors, cultured for 6 days, and mRNA levels of Sost and Shn3 were measured by qPCR analysis. Values represent mean±SD by an unpaired two-tailed Student's t-test and one-way ANOVA test. ns, not significant.

FIGS. 4A-4J show WNT-modulating gene silencers increase bone accrual in mice. A single dose (5×10¹³ vg/kg) of rAAV9.DSS carrying amiR-ctrl, amiR-shn3, amiR-sost, or amiR-sost/shn3 were i.v. injected into three-month-old male mice. 2 months later, mRNA levels of Shn3 and Sost (FIG. 4A) and β-catenin target genes Axin2 and Lef1 (FIG. 4B) were assessed in the tibial bone RNA and normalized to Gapdh (FIG. 4A, n=8/group). FIGS. 4C, 4D and 4G show femoral trabecular bone mass was assessed by microCT. Representative 3D-reconstruction (FIG. 4C) and relative quantification (FIG. 4D) are displayed (n=8/group). Trabecular bone volume/total volume (Tra.BV/TV), trabecular number (Tra.N), trabecular thickness (Tra.Th), and cortical thickness (Cort.Th) were measured. Scale bar, 1 mm. Values represent mean±SD by an unpaired two-tailed Student's t-test and one-way ANOVA test. ns, not significant. FIGS. 4E-4F show 1-month-old TCF/LEF1-GFP reporter mice (gray box) were i.v. injected with a single dose of rAAV9 carrying amiR-ctrl or amiR-sost/shn3 (5×10¹³ vg/kg) and two weeks later, mRNA levels of Opg and RankL in the tibia were measured by RT-PCR and normalized to b-actin (FIG. 4A, n=5/group). FIG. 4F shows bone accrual in the epiphyseal areas of humerus, hindlimb, and vertebrae was assessed by X-radiography (red arrows). Alternatively, mice were i.p. injected with OPG-Fc (1 mg/kg) weekly (n=3/group, FIG. 4F). FIG. 4H shows that dynamic histomorphometry was performed in the metaphysis of treated femurs and representative calcein/alizarin red labeling (FIG. 4H, left) and relative histomorphometric quantification of BFR/BS and MAR (FIG. 4H, right). FIG. 41 shows relative histomorphometric quantification of N.OC/B.Pm and ES/BS (n=10/group).

FIGS. 5A-5I show WNT-modulating gene silencers reverse bone loss in osteoporosis. FIG. 5A shows a diagram of the study and treatment methods. FIGS. 5B-5F show the sham or ovariectomized (OVX) surgery was performed on 3-month-old female mice and 6 weeks later, a single dose (5×10¹³ vg/kg) of rAAV9.DSS carrying amiR-ctrl, amiR-shn3, amiR-sost, or amiR-sost/shn3 was i.v. injected. 7 weeks after injection, mRNA levels of Shn3 and Sost in the tibial bone RNA are displayed after normalization to Bact (n=9/group) (FIG. 5B). Femoral trabecular bone mass was assessed by microCT. Representative 3D-reconstruction (FIG. 5C) and relative quantification (FIG. 5D) are displayed (n=5-10/group). Representative images of calcein/alizarin red labeling and relative histomorphometric quantification of BFR/BS and MAR (FIG. 5E). Arrows indicate the distance between calcein and alizarin red labeling. (FIGS. 5F-5I) A single dose (5×10¹³ vg/kg) of rAAV9.DSS carrying amiR-ctrl, amiR-shn3, amiR-sost, or amiR-sost/shn3 was i.v. injected into 18-month-old male mice. 2 months later, mRNA levels of Shn3 and Sost in the tibia bone RNA were assessed by qPCR and normalized to hprt (n=6/group) (FIG. 5F). Trabecular bone mass in femurs (FIGS. 5G and 5H) and lumbar vertebrae (L4, J) were assessed by microCT. Representative 3D-reconstruction (FIG. 5H) and relative quantification (FIGS. 5G and 51) are displayed (n=8-10/group). Scale bars: 1 mm. Scale bars: 1 mm. Values represent mean±SD by an unpaired two-tailed Student's t-test and one-way ANOVA test (FIGS. 5B, 5D, 5E, 5F, 5G, and 5I). ns, not significant.

FIG. 6 shows WNT-modulating dual gene silencers reverse bone loss in osteoporosis. Sham or OVX surgery was performed on 3-month-old female mice and 6 weeks later, a single dose (5×10¹³ vg/kg) of rAAV9.DSS carrying amiR-ctrl, amiR-shn3, amiR-sost, or amiR-sost/shn3 was i.v. injected. 7 weeks after injection, femoral trabecular thickness was assessed by microCT (n=5-10/group). Values represent mean±SD by an unpaired two-tailed Student's t-test and one-way ANOVA test. ns, not significant.

FIGS. 7A-7H show WNT-modulating AAV gene silencers promote bone regeneration in uni-cortical bone defect. FIG. 7A shows a diagram of the study and treatment methods. FIGS. 7B-7H show a single dose (5×10¹³ vg/kg) of rAAV9.DSS-amiR-ctrl, amiR-shn3, amiR-sost, or amiR-sost/shn3 was i.v. injected into 2-month-old female mice. 2 weeks following the injection, a 3 mm-length of uni-cortical bone defect was generated on the lateral aspect of the left femur (FIG. 7B). 2 weeks after the surgery, EGFP expression on the cryo-sectioned femurs was assessed by fluorescence microscopy (FIG. 7C). Scale bars: 500 μm. mRNA levels of Shn3 and Sost in the tibial bone RNA are displayed after normalization to Actb. Bone regeneration in the uni-cortical defect areas was assessed by microCT. Representative sagittal section images (FIG. 7D, left) and relative quantification of bone volume and region of interest (ROI, FIG. 7D, right) are displayed (n=8-11/group). Representative H&E-(FIG. 7E), toluidine blue-(FIG. 7F, top), and TRAP (FIG. 7F, bottom)-stained longitudinal sections of femurs in the areas of uni-cortical bone defect and relative quantification of osteoblast surface per bone surface (Ob.S/BS) (n=5/group) and osteoclast numbers per bone surface (Oc.N/B.S) (n=6/group) (FIG. 7G) were displayed. Scale bars: FIG. 7E, 200 μm; FIG. 7F, 100 μm. Values represent mean±SD by an unpaired two-tailed Student's t-test and one-way ANOVA test. ns, not significant.

FIGS. 8A-8J show WNT-modulating AAV gene silencers promote bone fracture union. Femoral osteotomy and intramedullary fixation were performed on 3-month-old female mice 2 weeks after the i.v. injection of rAAV9.DSS vectors (5×10¹³ vg/kg). To monitor AAV-transduced cells in the fracture areas, EGFP expression on the cryo-sectioned femurs was assessed by fluorescence microscopy 1, 2, and 4 weeks postoperatively (FIG. 8A). mRNA levels of shn3, sost (FIG. 8B) and b-catenin target gene Axin2 (FIG. 8C) in the tibial bone RNA were assessed and normalized to Gapdh (n=7-8/group). Trabecular bone mass of contralateral femurs was assessed by microCT (n=6-9/group) (FIG. 8D). Representative X-radiography and microCT images of the injured femurs at different time points after the fracture (FIG. 8E). Representative microCT images showing callus formation at the fracture sites (FIG. 8F) and relative quantification of callus total volume and callus mineral density (FIG. 8I) were displayed (n=4-7/group). Representative H&E-stained longitudinal sections of femurs at the fracture sites 6 weeks post-fracture (FIG. 8H). * indicates nonunion fibrous tissues. Union rate at the fracture sites was quantitated by microCT (FIG. 8I). Static histomorphometric quantification of osteoblast surface per bone surface (Ob.S/BS) (n=7-8/group) and osteoclast surface per bone surface (Oc.S/B.S) (n=5/group) were displayed (FIG. 8J). Scale bars: FIG. 8A, 25 m; FIG. 8E, 1 mm; FIG. 8F, 1 mm; FIG. 8H, 200 m. Values represent mean±SD by an unpaired two-tailed Student's t-test and one-way ANOVA test. ns, not significant.

FIGS. 9A-9D show effects of WNT-modulating AAV gene silencers on bone fracture healing. FIG. 9A shows a diagram of the study and treatment methods. FIG. 9B shows femoral osteotomy and intramedullary fixation were performed on 3-month-old female mice 2 weeks after the i.v. injection of rAAV9.DSS-amiR-ctrl, amiR-shn3, amiR-sost, or amiR-sost/shn3. To monitor AAV-transduced cells in the fracture areas, EGFP expression on the cryo-sectioned femurs was assessed by fluorescence microscopy 1, 2, 3, and 4 weeks postoperatively. Scale bar, 600 μm. FIG. 9C shows mRNA levels of β-catenin target gene Lef1 in the tibial bone RNA was assessed and normalized to Gapdh (n=6/group). FIG. 9D shows trabecular bone mass of contralateral femurs was assessed by microCT (n=7-8/group). Trabecular number (Tra.N), trabecular thickness (Tra.Th), and cortical thickness (Cort.Th).

FIGS. 10A-10M show development of a human skeletal organoid to treat critical-sized bone defect. FIG. 10A shows rAAV vectors (109 GC) were incubated with decellularized mouse bone or HA-scaffold for 1 hour at 37° C. and unbound rAAV vectors were removed by centrifugation. Vector titers were measured by ddPCR and normalized to PBS control. FIG. 10B shows mouse and human BMSCs were treated with PBS or rAAV9.DSS.egfp (5×10⁶ MOI) and 2 days later, EGFP expression was assessed by fluorescence microscopy. Scale bar, 500 μm. FIG. 10C shows PBS or rAAV9.DSS.egfp (4×10¹¹ GC) was incubated with freshly harvested human bone tissue and 2 days later, EGFP expression was measured by qPCR analysis and normalized to hprt (n=4/group). FIG. 10D shows a diagram of the study and treatment methods. FIG. 10E shows the hydroxyapatite (HA)-scaffold was incubated with rAAV9.egfp, rAAV9.DSS.egfp, or rAAV9.D14.egfp and 1 hour later, mouse BMSCs were cultured on the AAV-treated HA-scaffold. 2 days later, EGFP expression was assessed by immunoblotting with anti-EGFP antibody (left) and fluorescence microscopy (right). Scale bar, 500 μm (top) and 100 m (bottom). FIG. 10F shows human BMSCs were cultured on the AAV-treated HA-scaffold and 2 days later, EGFP expression was assessed by fluorescence microscopy (left) and qPCR analysis (right). Scale bar, 100 μm (top) and 25 μm (bottom). FIG. 10F shows human BMSCs were cultured on the AAV-treated HA-scaffold and 2 days later, EGFP expression was assessed by fluorescence microscopy (left) and qPCR analysis (right). Scale bar, 100 μm (top) and 25 m (bottom). FIG. 10G shows mRNA levels of BGLAP and SHN3 in freshly harvested human bone tissue (n=6/group). FIGS. 10H and 101 show human BMSCs were transduced with lentiviruses expressing vector control (Vec), SHN3, control-shRNA (Sh-con), or Shn3-shRNA (Sh-shn3), cultured under osteogenic conditions, and mRNA levels of SHN3 and IBSP were measured by qPCR analysis. Alternatively, mineralization activity was assessed by alizarin red staining (n=3/group). FIG. 10J shows a diagram of the rAAV9.DSS construct containing a CBA promoter, hs-amiR-hSHN3, an Egfp reporter gene, and β-globin polyA sequence. FIG. 10K shows that human BMSCs were incubated with rAAV9.DSS carrying hs-amiR-ctrl or hs-amiR-hSHN3 for two days and cultured under osteogenic conditions for four days. EGFP expression and mRNA levels of SHN3, BGLAP, or IBSP were assessed by fluorescence microscopy (left) and RT-PCR analysis (right, n=4/group), respectively. Scale bar, 100 μm (top) and 25 m (bottom). FIGS. 10L and 10M show HA-scaffold was incubated with rAAV9.DSS carrying hs-amiR-ctrl or hs-amiR-hSHN3 and one hour later, human BMSCs were cultured on the AAV-treated HA-scaffold under osteogenic conditions for two days. The treated HA-scaffold was implanted into the interscapular fat pads of immune-compromised SCID mice and four weeks later, bone anabolic activity was assessed by microCT. Representative 2D images and relative quantification are displayed (n=5/group). Non-treated HA-scaffold was used as a negative control (HA only, FIG. 10L). Alternatively, longitudinal section of HA-scaffold was stained for H&E (top), trichrome (middle), and BGLAP (bottom). Values represent mean±SD by an unpaired two-tailed Student's t-test (FIGS. 10K and 10L). Values represent mean±SD by an unpaired two-tailed Student's t-test (FIGS. 10C, 10F., 10G, 10H, and 10I) and one-way ANOVA test (FIG. 10A). ns, not significant.

FIGS. 11A and 11B show attachment of WNT-modulating AAVs to allograft and HA-scaffold. FIG. 11A shows representative pictures of mouse decellularized bone graft and HA-scaffold. FIG. 11B shows HA-scaffold was incubated with rAAV9.egfp, rAAV9.DSS.egfp, or rAAV9.D14.egfp and 1 hour later, mouse BMSCs were cultured on the AAV-treated HA-scaffold. 2 days later, EGFP mRNA levels were measured by qPCR analysis. Values represent mean±SD by an unpaired two-tailed Student's t-test and one-way ANOVA test.

FIGS. 12A-12J show local delivery of WNT-modulating AAV gene silencers promotes healing of critical-sized bone defect. FIGS. 12A-12C show either PBS-treated or rAAV9.DSS.egfp-attached allogenous bone (AB) was implanted into the areas of critical-sized femoral bone defect, and 3 weeks later, EGFP expression in individual tissues was monitored by IVIS-100 optical imaging (FIG. 12A) and fluorescence microscopy (FIGS. 12B and 12C). As a positive control, a single dose of rAAV9.DSS.egfp (5×10¹³ vg/kg) was i.v. injected (n=3/group). FIGS. 12D and 12F show implantation of the rAAV9.DSS-attached AB (isograft) into the areas of critical-sized femoral bone defect was performed in 3-month-old male mice and 12 weeks later, unionization of the implanted AB to the host bone was assessed by X-radiography and microCT. As a positive control, autogenous bone graft (ABG) was implanted into the injured sites. Representative images (FIG. 12D) and percentage of total bridging to autogenous bone graft (FIG. 12E) were displayed (n=15/group). H&E-stained longitudinal sections in the areas of critical-sized femoral bone defect (FIG. 12F). FIGS. 12G-12H show implantation of AB into the areas of critical-sized femoral bone defect was performed in 3-month-old male mice 2 weeks after a single dose (5×10¹³ vg/kg) of i.v. injection of rAAV9.DSS-amiR-ctrl, amiR-shn3, amiR-sost, or amiR-sost/shn3. 12 weeks later, unionization of the implanted AB to the host bone was assessed by microCT. Representative images (FIG. 12G) and percentage of total bridging (FIG. 12H) were displayed (n=5-6/group). FIG. 121 shows implantation of isograft into the osteotomy sites was performed in 3-month-old male mice two weeks after a single dose (5×10¹³ vg/kg) of i.v. injection of rAAV9.DSS carrying amiR-ctrl, amiR-shn3, amiR-sost, or amiR-sost/shn3. Twelve weeks later, total bridging of the implanted isograft to the host bone was assessed by microCT. Representative images (FIG. 121) and percentage of total bridging (FIG. 12J) are displayed (n=5-6/group). Scale bar, 500 μm. Scale bar: FIG. 12B, 25 m; FIG. 12C, 100 m; FIG. 12D, 1 mm; FIG. 12E top, 400 m; FIG. 12E bottom, 100 m; FIGS. 121 and 12J, 1 mm. Values represent mean±SD by an unpaired two-tailed Student's t-test and one-way ANOVA test.

FIGS. 13A-13C show the effects of WNT-modulating gene silencers on healing of critical-sized bone defect. FIG. 13A shows the preparation of allogenous bone attached with rAAV.DSS vectors. Allogenous bone was treated with DSS.rAAV9 (2.5×10¹¹ GC) for 30 min. FIG. 13B shows either PBS-treated or rAAV9.DSS.egfp-attached allogenous bone was implanted into the areas of critical-sized femoral bone defect, and 3 weeks later, EGFP expression in whole body was monitored by IVIS-100 optical imaging. As a positive control, a single dose of rAAV9.DSS.egfp (5×10¹³ vg/kg) was i.v. injected (n=3/group). FIG. 13C shows the implantation of allogenous bone into the areas of critical-sized femoral bone defect was performed in 3-month-old male mice 2 weeks after a single dose (5×10¹³ vg/kg) of i.v. injection of rAAV9.DSS-amiR-ctrl, amiR-shn3, amiR-sost, or amiR-sost/shn3. 12 weeks later, H&E staining was performed on the longitudinal sections of the injured femurs to assess unionization of the implanted AB to the host bone. HB: host bone, AB: allogenous bone. Scale bars: top, 400 m; bottom, 100 μm.

FIGS. 14A-14G show WNT-modulating gene silencers promote bone regeneration in uni-cortical bone defect. FIG. 14A shows pictures showing the femurs with uni-cortical bone defect. FIG. 14B shows diagram of the study and treatment methods. FIG. 14C shows 2-month-old female mice were i.v. injected with a single dose of rAAV9.DSS.egfp (5×10¹³ vg/kg) and two weeks later, a 3 mm-length of uni-cortical bone defect was generated on the lateral aspect of the left femur. GFP expression in the cryosectioned femurs was monitored by fluorescence microscopy two weeks post-surgery (n=3/group). Scale bars: 500 μm. FIGS. 14D-14G show two weeks after i.v. injection with a single dose (5×10¹³ vg/kg) of rAAV9.DSS-amiR-ctrl, amiR-shn3, amiR-sost, or amiR-sost/shn3, a 3 mm-length of uni-cortical bone defect was generated on the lateral aspect of the left femur. Bone regeneration in the areas of uni-cortical defect was assessed by microCT. Representative sagittal section images (FIG. 14D, left) and relative quantification of bone volume and region of interest (ROI, FIG. 14D, right) are displayed (n=8/group). Representative toluidine blue-(FIG. 14E, top), TRAP (FIG. 14E, bottom), and trichrome (FIG. 14G)-stained longitudinal sections of femurs in the areas of uni-cortical bone defect and relative quantification of Ob.S/BS and Oc.N/B.S are displayed (FIG. 14F, n=8/group). Ob.S/BS, osteoblast surface per bone surface; Oc.N/B.S, osteoclast numbers per bone surface. Values represent mean±SD by an unpaired two-tailed Student's t-test and one-way ANOVA test (FIG. 14D, 14F). ns, not significant.

FIG. 15A-15I show WNT-modulating gene silencers promote bone fracture healing. FIG. 15A shows femoral osteotomy and intramedullary fixation were performed on 3-month-old female mice two weeks after i.v. injection of rAAV9.DSS.egfp (5×10¹³ vg/kg). To monitor AAV-transduced cells in the fracture areas, EGFP expression on the cryo-sectioned femurs was assessed by fluorescence microscopy 1, 2, and 4 weeks postoperatively. FIGS. 15B-15I show 3-month-old female mice were i.v. injected with rAAV9.DSS carrying amiR-ctrl, amiR-shn3, amiR-sost, or amiR-sost/shn3 and femoral osteotomy and intramedullary fixation were performed two weeks post-injection. TRAP staining was performed in the metaphysis of treated femurs (FIG. 15B). Six weeks after fracture, mRNA levels of Shn3 and Sost (FIG. 15C) and Axin2 (FIG. 15D) in the tibia were assessed and normalized to Gapdh (n=8/group). Trabecular bone mass of contralateral femurs was assessed by microCT (FIG. 15E, n=8/group). Representative microCT images showing callus formation at the fracture sites (FIG. 15F, left) and relative quantification of callus total volume (FIG. 15F, right) are displayed (n=6-8/group). Union rate at the fracture sites was quantitated by microCT (FIG. 15G). Representative H&E-stained longitudinal sections of femurs at the fracture sites six weeks post-fracture. *indicates nonunion fibrous tissues (FIG. 15H). Histomorphometric quantification of Ob.S/BS and Oc.S/B.S are displayed (n=8/group, FIG. 15I). Scale bars: FIG. 15A, 25 m; FIG. 15F, 1 mm; FIG. 15H, 200 m. Values represent mean±SD by an unpaired two-tailed Student's t-test and one-way ANOVA test (FIGS. 15C-G, 15I). ns, not significant.

FIG. 16 shows the schematic diagram showing the molecular mechanism of SHN3 and SOST in the WNT/β-catenin pathway.

FIGS. 17A and 17B show the characterization of bone-specific AAV vectors carrying WNT-modulating gene silencers in bone accrual. FIG. 17A shows pictures (top) and X-ray images (bottom) showing the kinetics of tibial fracture healing of 2-month old wildtype mice. This experiment represented pictures of tibial bone fracture performed in FIG. 1C. FIG. 17B shows 1-month-old mice were treated with a single dose of rAAV.DSS vectors (5×10¹³ vg/kg) via intravenous (i.v.) injection or with OPG-Fc (1 mg/kg) via intraperitoneal (i.p.) injection and two weeks later, bone accrual was assessed by X-radiography. Boxes indicate the areas of increased bone accrual.

FIGS. 18A-18E show AAV-mediated silencing of Sost expression increases bone mass in mice. FIG. 18A shows a diagram of the rAAV9 construct containing a CMV enhancer/chicken β-actin promoter (CBA), amiR-ctrl, amiR-Sost1, or amiR-Sost2, an Egfp reporter gene (EGFP), β-globin polyA sequence (polyA), and terminal repeat (TR). FIG. 18B shows a single dose (5×10¹³ vg/kg) of rAAV9.egfp was i.v. injected into 12-week-old mice and 2 months later, EGFP expression was assessed by fluorescence microscopy of cryo-sectioned femurs. Scale bar, 250 m (left) and 25 μm (right). BM, bone marrow; M, muscle; Cb, cortical bone. FIGS. 18C-18E show a single dose (5×10¹³ vg/kg) of rAAV9.egfp carrying amiR-ctrl, amiR-Sost1, or amiR-Sost2 was i.v. injected into 12-week-old mice and 2 months later, Sost mRNA levels in the tibia of AAV-treated mice were assessed by RT-PCR (FIG. 18C). MicroCT analysis showing femoral bone mass in AAV-treated mice. Relative quantification (FIG. 18D) and representative 3D-reconstruction (FIG. 18E) are displayed. Trabecular bone volume/total volume (Tb. BV/TV), trabecular number (Tb. N), trabecular thickness (Tb.Th), and trabecular connective density (Tra. ConnDn) were measured. Scale bar, 500 μm. Values represent mean±SD by an unpaired two-tailed Student's t-test and one-way ANOVA test. ns, not significant.

FIGS. 19A-19C show Effects of WNT-modulating gene silencers on osteoclast differentiation and resorption activity in vitro. Bone marrow-derived monocytes (BMMs) harvested from 2-month-old wildtype mice were treated with M-CSF (20 ng/ml) and RANKL (10 ng/ml) for one day and then, transduced with rAAV9 carrying amiR-ctrl, amiR-shn3, amiR-sost, or amiR-shn3/sost (5×10⁶ MOI). AAV-transduced BMMs were cultured with M-CSF and RANKL for six days to differentiate them into mature osteoclasts. FIG. 19A shows expression of Shn3 and osteoclast genes, Acp5 and Ctsk, was assessed by RT-PCR and normalized to β-actin (n=4/group). FIGS. 19B and 19C show osteoclast differentiation and resorption activity were assessed by TRAP staining and resorption pit assay, respectively (n=4/group). Scale bars: 1 mm. Values represent mean±SD by an unpaired two-tailed Student's t-test and one-way ANOVA test. ns, not significant.

FIGS. 20A-20D show effects of WNT-modulating gene silencers on bone fracture healing. FIG. 20A shows a diagram of the study and treatment methods. FIG. 20B shows femoral osteotomy and intramedullary fixation were performed on 3-month-old female mice 2 weeks after the i.v. injection of rAAV9.DSS carrying amiR-ctrl, amiR-shn3, amiR-sost, or amiR-sost/shn3. To monitor AAV-transduced cells in the fracture areas, EGFP expression on the cryo-sectioned femurs was assessed by fluorescence microscopy 1, 2, 3, and 4 weeks postoperatively (n=3/group). Scale bar, 600 μm. Low magnification images of tibial bone fractures performed in FIG. 8A are shown. FIG. 20C shows mRNA levels of β-catenin target gene Lef1 in the tibial bone RNA was assessed and normalized to β-actin (n=8/group). FIG. 20D shows trabecular bone mass of contralateral femurs was assessed by microCT (n=8/group). Trabecular number (Tra.N), trabecular thickness (Tra.Th), and cortical thickness (Cort.Th).

FIGS. 21A-21D show effects of human SHN3 knockdown on osteoblast differentiation in vitro. FIG. 21A shows representative pictures of mouse decellularized bone graft and HA-scaffold. FIG. 21B shows HA-scaffold was incubated with rAAV9.egfp, rAAV9.DSS.egfp, or rAAV9.D14.egfp and 1 hour later, mouse BMSCs were cultured on the AAV-treated HA-scaffold. 2 days later, EGFP mRNA levels were measured by qPCR analysis. FIGS. 21C and 21D show human BMSCs were transduced with lentiviruses expressing vector control (Vec), SHN3, control-shRNA (Sh-con), or Shn3-shRNA (Sh-shn3), cultured under osteogenic conditions, and mRNA levels of SHN3 and IBSP were measured by RT-PCR analysis. Alternatively, mineralization activity was assessed by alizarin red staining (n=3/group). Values represent mean±SD by an unpaired two-tailed Student's t-test and one-way ANOVA test.

FIGS. 22A-22C show effects of humanized SHN3 gene silencer on bone formation. FIGS. 22A and 22B show that human BMSCs were incubated with PBS or rAAV9.DSS carrying hs-amiR-ctrl, hs-amiR-hSHN3-1, or hs-amiR-hSHN3-2 (10¹¹ GC) for two days and cultured under osteogenic conditions for six days. AAV's transduction was assessed using EGFP expression by fluorescence microscopy (FIG. 22A). Expression SHN3 and osteogenic genes was assessed by RT-PCR analysis and normalized to HPRT (FIG. 22B, n=4/group). FIG. 22C shows HA-scaffold was incubated with rAAV9.DSS carrying hs-amiR-ctrl or hs-amiR-hSHN3 and one hour later, human BMSCs were cultured on the AAV-treated HA-scaffold under osteogenic conditions for two days. The treated HA-scaffold was implanted into the interscapular fat pads of immune-compromised SCID mice and four weeks later, bone anabolic activity was assessed by X-radiography and microCT (n=5/group). Non-treated HA-scaffold was used as a negative control.

FIGS. 23A-23E show effects of WNT-modulating gene silencers on healing of critical-sized femoral defect. FIG. 23A shows a diagram of the study and treatment methods. FIG. 23B shows that decellularized isograft was implanted into the osteotomy site of left femurs in 3-month-old male mice two weeks after a single dose (5×10¹³ vg/kg) of i.v. injection of rAAV9.DSS-amiR-ctrl, amiR-shn3, amiR-sost, or amiR-sost/shn3. Twelve weeks later, H&E staining was performed on the longitudinal sections of the injured femurs to assess unionization of the implanted isograft to the host bone. (n=5-6/group). HB: host bone, GB: graft bone. Scale bars: top, 400 m; bottom, 100 μm. This experiment represented H&E staining images of the unionization sites performed in FIGS. 121 and 12J. FIG. 23C shows preparation of isograft attached with rAAV9.DSS vectors. Decellularized isograft was incubated with rAAV9.DSS (2.5×10¹¹ GC) for one hour. FIG. 23D shows that either PBS-treated or rAAV9.DSS.egfp-attached isograft was implanted into the osteotomy site of right femurs in 3-month-old male mice, and three weeks later, EGFP expression in whole body was monitored by IVIS-100 optical imaging. As a positive control, a single dose of rAAV9.DSS.egfp (5×10¹³ vg/kg) was i.v. injected (n=3/group). This experiment represented IVIS-100 optical imaging of whole body performed in FIG. 7A. FIG. 23E shows a diagram of the study and treatment methods.

### DETAILED DESCRIPTION OF INVENTION

Aspects of the disclosure relate to methods and compositions (e.g., isolated nucleic acids, rAAVs, etc.) for treating osteoporosis or critical-sized bone defect that when delivered to a subject are effective for modulating bone metabolism and healing, for example by promoting or inhibiting bone formation and/or reversing bone loss. Accordingly, methods and compositions described by the disclosure are useful, in some embodiments, for the treatment of diseases and disorders associated with osteoporosis, bone fracture, and persistent nonunion of bone fracture. Bone remodeling is a continuous bone replacement regulated by serial action between bone-forming osteoblasts and bone-resorbing osteoclasts, and crucial for utmost bone quality and proper fracture healing. While long-term treatment with antiresorptive drugs impairs bone remodeling in patients with osteoporosis and/or fracture, bone remodeling is relatively active in the presence of anabolic drugs, and therefore, anabolic drugs are considered as promising therapeutic interventions for osteoporosis and fracture. WNT signaling is known as a pivotal regulator of bone formation that increases bone mass and strength via augmented osteogenesis.

A number of WNT antagonists have been identified to inhibit osteogenesis and bone formation. Sclerostin, an antagonist of the WNT signaling pathway competing with WNT ligands, is the most investigated WNT modulator, and anti-sclerostin antibody is available in clinical practice. Romosozumab, a humanized monoclonal antibody, significantly increased bone mineral density with increase in levels of bone-formation markers over the first 6 to 9 months of treatment in postmenopausal women. Sclerostin is a secreted factor produced by osteocytes that interferes with the engagement of WNTs with the WNT receptor Frizzled by binding to co-receptors of WNTs, low-density lipoprotein receptor-related proteins 5 and 6 (LRP5/6). Its deletion leads to high bone mass phenotype due to enhanced bone formation in mice and humans. Notably, sclerostin has been recognized as a new target for therapeutic intervention in patients with osteoporosis, as anti-sclerostin antibody promotes bone formation. However, since bone formation markers wanes over time following the treatment, longer than 1 year treatment is not recommended.

Schnurri-3 (SHN3) is the intracellular adaptor protein that downregulates the expression of β-catenin downstream of WNT signaling in OBs. SHN3-deficiency results in a progressive increase in bone mass and its effect is specific to OBs without any phenotypes in non-skeletal tissues. The bone formed in shn3^{-/-} mice is mature lamellar bone with normal biomechanical properties. These properties together make SHN3 inhibition an attractive approach to promote bone formation to treat osteoporosis. Despite several studies showing that inhibition of sclerostin or SHN3 could promote bone formation early in fracture healing, its therapeutic efficacy is still unpredictable.

Additionally, the healing of critical sized skeletal defects remains one of the most challenging problems in orthopedic management since these defects are unable to heal without interventions such as insertion of a bone graft. Similarly, over 15% of complex trauma results in persistent nonunion of a skeletal fracture. Thus, improving patient outcomes in these scenarios hinges on accelerating skeletal healing to speed recovery of mobility.

The present disclosure describes that bone-homing rAAV-mediated silencing of WNT antagonists enhance bone regeneration in osteoporosis and fracture healing. It was observed that the expression level of SOST is increased via a negative feedback mechanism of SHN3. To further enhance WNT signaling, dual silencer molecules that inhibit both SHN3 and SOST were produced. The present disclosure also provides a gene therapy approach that promotes bone regeneration in order to treat a skeletal fracture with persistent non-union and/or critical sized skeletal defects using a bone-attached recombinant adeno-associated virus (rAAV). The present disclosure describes that the dual silencer increases bone formation more than single silencers of SHN3 or SOST in osteoporosis and fracture healing. Gene silencers to allogenous bone by utilizing the ability of bone homing rAAV were attached and bone healing efficiency in critical size bone defect was observed.

### Isolated Nucleic Acids

Compositions and methods for delivering a transgene (e.g. an inhibitory RNA, such as an shRNA, miRNA, etc.) to a subject are provided in the disclosure. The compositions typically comprise an isolated nucleic acid encoding a transgene (e.g., a protein, an inhibitory nucleic acid, etc.) capable of modulating bone metabolism. For example, in some embodiments, a transgene reduces expression of a target protein, such as a target protein associated with promoting or inhibiting bone formation.

“Bone metabolism” generally refers to a biological process involving bone formation and/or bone resorption. In some embodiments, bone metabolism involves the formation of new bone as produced by osteoblasts (OBs) and differentiated osteocytes, and/or mature bone tissue being resorbed by osteoclasts (OCs). OBs arise from the bone marrow derived mesenchymal cells that ultimately differentiate terminally into osteocytes. OB (and osteocyte) functions or activities include but are not limited to bone formation, bone mineralization, and regulation of OC activity. Decreased bone mass has been observed to result from inhibition of OB and/or osteocyte function or activity. Increased bone mass has been observed to result from increased OB and/or osteocyte function or activity. OCs arise from bone marrow-derived monocytes and in some embodiments have been observed to be controlled by signals from OBs. OC functions include bone resorption. In some embodiments, decreased bone mass has been observed to result from increased OC activity. In some embodiments, increased bone mass has been observed to result from inhibition of OC activity.

In some embodiments, an isolated nucleic acid or an rAAV as described by the disclosure comprises a transgene encoding at least one inhibitory nucleic acid (e.g., 1, 2, 3, 4, 5, 6, 7, 8, 9, 10, or more inhibitory nucleic acids). As used herein, the inhibitory nucleic acid targets at least one WNT/β-catenin pathway antagonist. In some embodiments, the inhibitory nucleic acid is a WNT signaling modulator. In some embodiments, the inhibitory nucleic acid targets Schnurri-3 (SHN3). In some embodiments, the inhibitory nucleic acid targets Sclerostin (SOST). In some embodiments, the inhibitory nucleic acid can target any protein that serves as antagonists of the Wnt signaling pathway. In some embodiments, the inhibitory nucleic acid as described herein inhibits the expression activity, and/or function of Wnt antagonists.

In some embodiments, the isolated nucleic acid or an rAAV as described by the disclosure may comprises a transgene encoding a bone metabolism modulating agent. As used herein, a “bone metabolism modulating agent” refers to a molecule (a nucleic acid or protein encoded by a nucleic acid, e.g., a transgene) that either induces or inhibits bone formation or deposition, for example by increasing or decreasing expression, activity, and/or function of proteins, cells, etc., that are involved in bone formation or bone resorption. Generally, a bone metabolism modulating agent can be a peptide, protein, or an interfering nucleic acid (e.g., dsRNA, siRNA, shRNA, miRNA, artificial miRNA, etc.). In some embodiments, a bone metabolism modulating agent is a bone formation inducing agent. In some embodiments, a bone metabolism modulating agent is a bone formation inhibiting agent.

A “bone formation inducing agent” refers to a molecule that promotes bone synthesis either by promoting OB and/or osteocyte (OCY) differentiation or activity and/or by inhibiting OC activity. In some embodiments, a bone formation inducing agent is a nucleic acid (e.g., RNAi oligonucleotide or miRNA oligonucleotide or antisense oligonucleotide) or protein encoded by a nucleic acid (e.g., a transgene) that promotes OB and/or osteocyte function or activity (e.g., bone formation, mineralization, regulation of osteoclast activity or function, etc.). In some embodiments, examples of bone formation inducing agents that promote OB and/or osteocyte activity or function include but are not limited to parathyroid hormone (PTH), PTH-related protein (PTHrP), deglycase DJ1. In some embodiments, a bone formation inducing agent is an inhibitory nucleic acid that inhibits OC differentiation or activity, such as an inhibitory nucleic acid that targets sclerostin (SOST), schnurri-3 (SHN3), cathepsin K (CTSK), etc.

In some embodiments, an isolated nucleic acid encodes one or more (e.g., 1, 2, 3, 4, 5, 6, 7, 8, 9, 10, or more) inhibitory nucleic acids, for example dsRNA, siRNA, shRNA, miRNA, artificial microRNA (ami-RNA), etc.). Generally, an inhibitory nucleic acid specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of a gene encoding a gene product (e.g., a protein) associated with bone metabolism (e.g., SOST, SHN3 etc.). As used herein “continuous bases” refers to two or more nucleotide bases that are covalently bound (e.g., by one or more phosphodiester bond, etc.) to each other (e.g. as part of a nucleic acid molecule). In some embodiments, the at least one inhibitory nucleic acid is about 50%, about 60% about 70% about 80% about 90%, about 95%, about 99% or about 100% identical to the two or more (e.g., 2, 3, 4, 5, 6, 7, 8, 9, 10, 11, 12, 13, 14, 15, 16, 17, 18, 19, 20, 21, 22, 23, 24, 25, or more) continuous nucleotide bases of a gene encoding a gene product (e.g., a protein) associated with bone metabolism (e.g., SOST, SHN3, etc.).

A “microRNA” or “miRNA” is a small non-coding RNA molecule capable of mediating transcriptional or post-translational gene silencing. Typically, miRNA is transcribed as a hairpin or stem-loop (e.g., having a self-complementarity, single-stranded backbone) duplex structure, referred to as a primary miRNA (pri-miRNA), which is enzymatically processed (e.g., by Drosha, DGCR8, Pasha, etc.) into a pre-miRNA. The length of a pri-miRNA can vary. In some embodiments, a pri-miRNA ranges from about 100 to about 5000 base pairs (e.g., about 100, about 200, about 500, about 1000, about 1200, about 1500, about 1800, or about 2000 base pairs) in length. In some embodiments, a pri-miRNA is greater than 200 base pairs in length (e.g., 2500, 5000, 7000, 9000, or more base pairs in length.

Pre-miRNA, which is also characterized by a hairpin or stem-loop duplex structure, can also vary in length. In some embodiments, pre-miRNA ranges in size from about 40 base pairs in length to about 500 base pairs in length. In some embodiments, pre-miRNA ranges in size from about 50 to 100 base pairs in length. In some embodiments, pre-miRNA ranges in size from about 50 to about 90 base pairs in length (e.g., about 50, about 52, about 54, about 56, about 58, about 60, about 62, about 64, about 66, about 68, about 70, about 72, about 74, about 76, about 78, about 80, about 82, about 84, about 86, about 88, or about 90 base pairs in length).

Generally, pre-miRNA is exported into the cytoplasm, and enzymatically processed by Dicer to first produce an imperfect miRNA/miRNA* duplex and then a single-stranded mature miRNA molecule, which is subsequently loaded into the RNA-induced silencing complex (RISC). Typically, a mature miRNA molecule ranges in size from about 19 to about 30 base pairs in length. In some embodiments, a mature miRNA molecule is about 19, about 20, about 21, about 22, about 23, about 24, about 25, about 26, about 27, about 28, about 29, or 30 base pairs in length.

In some aspects, the disclosure provides isolated nucleic acids and vectors (e.g., rAAV vectors) that encode one or more artificial miRNAs. As used herein “artificial miRNA” or “amiRNA” refers to an endogenous pri-miRNA or pre-miRNA (e.g., a miRNA backbone, which is a precursor miRNA capable of producing a functional mature miRNA), in which the miRNA and miRNA* (e.g., passenger strand of the miRNA duplex) sequences have been replaced with corresponding amiRNA/amiRNA* sequences that direct highly efficient RNA silencing of the targeted gene, for example as described by Eamens et al. (2014), Methods Mol. Biol. 1062:211-224. For example, in some embodiments an artificial miRNA comprises a miR-33 pri-miRNA backbone into which a sequence encoding a bone metabolism modulating (e.g., bone formation inhibiting agent) miRNA has been inserted in place of the endogenous miR-33 mature miRNA-encoding sequence. In some embodiments, miRNA (e.g., an artificial miRNA) as described by the disclosure comprises a miR-33backbone sequence. In some embodiments, miRNA (e.g., an artificial miRNA) as described by the disclosure can comprise any suitable miRNA.

In some embodiments, the present disclosure provides an isolated nucleic acid comprising a transgene encoding an artificial microRNA targeting the SHN3 gene (GeneID: 59269), which encodes the Schnurri-3 protein. The Schnurri-3 (SHN3) protein is a transcription factor that regulates NK-1<0 protein expression and immunoglobulin and T-cell receptor antibody recombination. In some embodiments, the SHN3 gene is represented by the NCBI Accession Number NM_001127714.2 or NM_024503.5. In some embodiments, the SHN3 protein is represented by the NCBI Accession Number NP_001121186.1 or NP_078779.2.

In some aspects, the disclosure relates to an isolated nucleic acid comprising a transgene encoding an artificial microRNA is used to reduce SHN3 expression (e.g., expression of one or more gene products from an SHN3 gene). In some embodiments, the inhibitory nucleic acid is encoded by a nucleic acid comprising the sequence set forth in any one of SEQ ID NOs: 4, 5, 6, 9 and 10.

In some embodiments, the present disclosure provides an isolated nucleic acid comprising a transgene encoding an artificial microRNA targeting the SOST gene (GeneID: 50964), which encodes the sclerostin protein. In some embodiments, the inhibitory nucleic acid is encoded by a nucleic acid comprising the sequence set forth in SEQ ID NO: 7.

In some embodiments, an artificial microRNA targets (e.g., binds to, or comprises a region of complementarity with) at least 6 continuous nucleotides of a SHN3 or SOST gene. In some embodiments, an artificial microRNA targets (e.g., binds to, or comprises a region of complementarity with) between 6 and 30 continuous nucleotides of a SHN3 or SOST gene. In some embodiments, an artificial microRNA targets between 12-24 continuous nucleotides of a SHN3 or SOST gene. In some embodiments, an artificial microRNA targets between 9-27 continuous nucleotides of the SHN3 gene. In some embodiments, an artificial microRNA targets at least 6, 7, 8, 9, 10, 11, 12, 13, 14, 15, 16, 17, 18, 19, 20, 21, 22, 23, 24, 25, 26, 27, 28, 29, or 30 continuous nucleotides of a SHN3 or SOST gene.

In some embodiments, an artificial microRNA is between 6-50 nucleotides in length. In some embodiments, an artificial microRNA is between 8-24 nucleotides in length. In some embodiments, an artificial microRNA is between 12-36 nucleotides in length. In some embodiments, an artificial microRNA is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, an isolated inhibitory nucleic acid decreases expression of a target gene by between 50% and 99% (e.g., any integer between 50% and 99%, inclusive). In some aspects, an isolated inhibitory nucleic acid decreases expression of a target gene by between 75% and 90%. In some aspects, an isolated inhibitory nucleic acid decreases expression of a target gene by between 80% and 99%. In some embodiments, an isolated inhibitory nucleic acid decreases expression of a SHN3 gene by between 50% and 99% (e.g., any integer between 50% and 99%, inclusive). In some embodiments, an isolated inhibitory nucleic acid decreases expression of a SHN3 or SOST gene by between 75% and 90%. In some aspects, an isolated inhibitory nucleic acid decreases expression of a SHN3 or SOST gene by between 80% and 99%.

A region comprising a transgene (e.g., a second region, third region, fourth region, etc.) may be positioned at any suitable location of the isolated nucleic acid. The region may be positioned in any untranslated portion of the nucleic acid, including, for example, an intron, a 5′ or 3′ untranslated region, etc.

In some cases, it may be desirable to position the region (e.g., the second region, third region, fourth region, etc.) upstream of the first codon of a nucleic acid sequence encoding a protein (e.g., a protein coding sequence). For example, the region may be positioned between the first codon of a protein coding sequence) and 2000 nucleotides upstream of the first codon. The region may be positioned between the first codon of a protein coding sequence and 1000 nucleotides upstream of the first codon. The region may be positioned between the first codon of a protein coding sequence and 500 nucleotides upstream of the first codon. The region may be positioned between the first codon of a protein coding sequence and 250 nucleotides upstream of the first codon. The region may be positioned between the first codon of a protein coding sequence and 150 nucleotides upstream of the first codon.

In some cases (e.g., when a transgene lacks a protein coding sequence), it may be desirable to position the region (e.g., the second region, third region, fourth region, etc.) upstream of the poly-A tail of a transgene. For example, the region may be positioned between the first base of the poly-A tail and 2000 nucleotides upstream of the first base. The region may be positioned between the first base of the poly-A tail and 1000 nucleotides upstream of the first base. The region may be positioned between the first base of the poly-A tail and 500 nucleotides upstream of the first base. The region may be positioned between the first base of the poly-A tail and 250 nucleotides upstream of the first base. The region may be positioned between the first base of the poly-A tail and 150 nucleotides upstream of the first base. The region may be positioned between the first base of the poly-A tail and 100 nucleotides upstream of the first base. The region may be positioned between the first base of the poly-A tail and 50 nucleotides upstream of the first base. The region may be positioned between the first base of the poly-A tail and 20 nucleotides upstream of the first base. In some embodiments, the region is positioned between the last nucleotide base of a promoter sequence and the first nucleotide base of a poly-A tail sequence.

In some cases, the region may be positioned downstream of the last base of the poly-A tail of a transgene. The region may be between the last base of the poly-A tail and a position 2000 nucleotides downstream of the last base. The region may be between the last base of the poly-A tail and a position 1000 nucleotides downstream of the last base. The region may be between the last base of the poly-A tail and a position 500 nucleotides downstream of the last base. The region may be between the last base of the poly-A tail and a position 250 nucleotides downstream of the last base. The region may be between the last base of the poly-A tail and a position 150 nucleotides downstream of the last base.

It should be appreciated that in cases where a transgene encodes more than one miRNA, each miRNA may be positioned in any suitable location within the transgene. For example, a nucleic acid encoding a first miRNA may be positioned in an intron of the transgene and a nucleic acid sequence encoding a second miRNA may be positioned in another untranslated region (e.g., between the last codon of a protein coding sequence and the first base of the poly-A tail of the transgene).

In some embodiments, the transgene further comprises a nucleic acid sequence encoding one or more expression control sequences (e.g., a promoter, etc.). Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931.; and Klump, H et al., Gene Therapy, 2001; 8: 811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is an enhanced chicken β-actin promoter. In some embodiments, a promoter is a U6 promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor a-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, a tissue-specific promoter is a bone tissue-specific promoter. Examples of bone tissue-specific promoters include but are not limited to promoters of osterix, osteocalcin, type 1 collagen al, DMP1, cathepsin K, Rank, etc.

Aspects of the disclosure relate to an isolated nucleic acid comprising more than one promoter (e.g., 2, 3, 4, 5, or more promoters). For example, in the context of a construct having a transgene comprising a first region encoding a protein and an second region encoding an inhibitory RNA (e.g., miRNA), it may be desirable to drive expression of the protein coding region using a first promoter sequence (e.g., a first promoter sequence operably linked to the protein coding region), and to drive expression of the inhibitory RNA encoding region with a second promoter sequence (e.g., a second promoter sequence operably linked to the inhibitory RNA encoding region). Generally, the first promoter sequence and the second promoter sequence can be the same promoter sequence or different promoter sequences. In some embodiments, the first promoter sequence (e.g., the promoter driving expression of the protein coding region) is a RNA polymerase III (polIII) promoter sequence. Non-limiting examples of poIIII promoter sequences include U6 and H1 promoter sequences. In some embodiments, the second promoter sequence (e.g., the promoter sequence driving expression of the inhibitory RNA) is a RNA polymerase II (polII) promoter sequence. Non-limiting examples of polII promoter sequences include T7, T3, SP6, RSV, and cytomegalovirus promoter sequences. In some embodiments, a polIII promoter sequence drives expression of an inhibitory RNA (e.g., miRNA) encoding region. In some embodiments, a polII promoter sequence drives expression of a protein coding region.

In some embodiments, an isolated inhibitory nucleic acid reduces bone loss by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, an isolated inhibitory nucleic acid improves bone healing by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

As used herein, “control” can refer to any subjects who do not have, are not suspected of, or are at risk of developing a disease or disorder associated with osteoporosis or critical-sized bone defect or suffering from bone fractures. “Control” can refer to the same subject before receiving the treatment disclosed herein. The control does not have one or more signs or symptoms of osteoporosis, critical-sized bone defect or bone fractures. The control can be a normal, healthy subject.

Recombinant AAVs (rAAVs)

Adeno-associated virus (AAV) is a small (26 nm) non-enveloped parvovirus with a single-stranded genome of approximately 4.7 kb in length. High transduction efficiency, persistent transgene expression, and lack of post-infection immunogenicity and pathogenicity make AAV an attractive viral vector for use in gene therapy. The AAV genome encodes regulatory (Rep) and structural capsid (Cap) proteins and is flanked by two inverted terminal repeats (ITRs). Replacement of the Rep and Cap genes with a transgene of interest produces a replication-defective recombinant AAV (rAAV) genome that can transduce target tissues as a potent vector. Second-generation self-complementary AAVs (scAAVs), which package a double-stranded DNA genome, can be engineered to bypass the rate-limiting second-strand synthesis step required for rAAV transgene expression. As a result, scAAV vectors have enhanced transduction efficacies in vitro and in vivo. However, AAV-based gene therapies for bone and joint disorders is limited.

The present disclosure provides that bone-homing rAVV-mediated silencing of WNT antagonists enhance bone regeneration in osteoporosis and fracture healing. The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more proteins and/or inhibitory nucleic acids (e.g., shRNA, miRNAs, etc.) comprising a nucleic acid that targets an endogenous mRNA of a subject. The transgene may also comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16(10):1648-1656.

As used herein, the term “self-complementary AAV vector” (scAAV) refers to a vector containing a double-stranded vector genome generated by the absence of a terminal resolution site (TR) from one of the ITRs of the AAV. The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle. The instant invention is based, in part, on the recognition that DNA fragments encoding RNA hairpin structures (e.g. shRNA, miRNA, and AmiRNA) can serve a function similar to a mutant inverted terminal repeat (mTR) during viral genome replication, generating self-complementary AAV vector genomes. For example, in some embodiments, the disclosure provides rAAV (e.g. self-complementary AAV; scAAV) vectors comprising a single-stranded self-complementary nucleic acid with inverted terminal repeats (ITRs) at each of two ends and a central portion comprising a promoter operably linked with a sequence encoding a hairpin-forming RNA (e.g., shRNA, miRNA, ami-RNA, etc.). In some embodiments, the sequence encoding a hairpin-forming RNA (e.g., shRNA, miRNA, ami-RNA, etc.) is substituted at a position of the self-complementary nucleic acid normally occupied by a mutant ITR.

“Recombinant AAV (rAAV) vectors” are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The instant disclosure provides a vector comprising a single, cis-acting wild-type ITR. In some embodiments, the ITR is a 5′ ITR. In some embodiments, the ITR is a 3′ ITR Generally, ITR sequences are about 145 bp in length. Preferably, substantially the entire sequences encoding the ITR(s) is used in the molecule, although some degree of minor modification of these sequences is permissible. The ability to modify ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). For example, an ITR may be mutated at its terminal resolution site (TR), which inhibits replication at the vector terminus where the TR has been mutated and results in the formation of a self-complementary AAV. Another example of such a molecule employed in the present disclosure is a “cis-acting” plasmid containing the transgene, in which the selected transgene sequence and associated regulatory elements are flanked by the 5′ AAV ITR sequence and a 3′ hairpin-forming RNA sequence. AAV ITR sequences may be obtained from any known AAV, including presently identified mammalian AAV types. In some embodiments, an ITR sequence is an AAV2, AAV3, AAV4, AAV5, AAV6, AAV7, AAV8, AAVrh8, AAV9, AAV10, and/or AAVrh10 ITR sequence.

In some embodiments, the rAAVs of the disclosure are pseudotyped rAAVs. For example, a pseudotyped AAV vector containing the ITRs of serotype X encapsidated with the proteins of Y will be designated as AAVX/Y (e.g. AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125. In some embodiments, an AAV capsid protein is of a serotype derived from a non-human primate, for example scAAV.rh8, AAV.rh39, or AAV.rh43 serotype. In some embodiments, an AAV capsid protein is of an AAV9 serotype.

The disclosure is based, in part, on rAAVs comprising capsid proteins that have increased tropism for bone tissue. In some embodiments, the capsid proteins are grafted to a bone-targeting peptide. A heterologous bone-targeting peptide may target OCs (e.g., specifically, or preferentially targets OCs relative to OBs) or OBs (e.g., specifically, or preferentially targets OBs relative to OCs). In some embodiments, a bone-targeting peptide is an (AspSerSer)₆ peptide, which may also be referred to as a DSS₆ peptide (e.g. SEQ ID NO: 11). Additional bone-targeting peptide such as a HABP-19 peptide (CTEPRR7EVATELTEPRR7EVATEL; SEQ ID NO: 12), which may be referred to as a HABP peptide, can also be used. In some embodiments, a bone-targeting peptide is an (Asp)₈0.14 peptide comprising 8-14 aspartic acid residues. Further examples of bone-targeting peptides include but are not limited to those described by Ouyang et al. (2009) Lett. Organic Chem 6(4):272-277.

In some embodiments, the rAAV as disclosed herein is attached to a hydroxyapatite (HA) scaffold. Grafting the bone-targeting peptide motif, ((AspSerSer)₆, DSS) onto the N-terminus of VP2 capsid protein (rAAV9.DSS-Nter) enables rAAVs such as rAAV9 to attach hydroxyapatite (HA). It has been reported that HA is a major inorganic component in the bone tissue, which improves bone-specific tropism of the rAAVs such as rAAV9, when systemically administered into mice.

As used herein, “grafting” refers to joining or uniting of one molecule with another molecule. In some embodiments, the term grafting refers to joining or uniting of at least two molecules such that one of the at least two molecules is inserted within another of at least two molecules. In some embodiments, the term grafting refers to joining or uniting of at least two polymeric molecules such that one of at least two molecules is appended to another of at least two molecules. In some embodiments, the term grafting refers to joining or uniting of one polymeric molecule (e.g., a nucleic acid, a polypeptide) with another polymeric molecule (e.g., a nucleic acid, a polypeptide). In some embodiments, the term grafting refers to joining or uniting of at least two nucleic acid molecules such that one of at least two molecules is appended to another of at least two nucleic acid molecules.

In some embodiments, the term grafting refers to joining or uniting of at least two nucleic acid molecules such that one of the at least two nucleic acid molecules is inserted within another of the at least two nucleic acid molecules. For example, it has been observed that targeting peptides may be grafted to certain loci of a nucleic acid encoding a VP2 AAV capsid protein. In some embodiments, a targeting peptide (e.g. a bone-targeting peptide) is inserted at a position corresponding to the position between the codons encoding Q588 and A589 and/or N587 and R588 of an AAV2 or AAV9 VP2 capsid protein. In some embodiments, a targeting peptide is inserted at a position between the codons encoding N587 and R588 of an VP3 capsid protein (or a position corresponding to such amino acid positions in AAV2 or AAV9). In some embodiments, a targeting peptide is inserted at a position between the codons encoding S452 and G453 of an VP1 capsid protein. Other potential positions may be N587 and R588.

In some embodiments, a nucleic acid formed through grafting (a grafted nucleic acid) encodes a chimeric protein. In some embodiments, a grafted nucleic acid encodes a chimeric protein, such that one polypeptide is effectively inserted into another polypeptide (e.g. not directly conjugated before the N-terminus or after the C-terminus), thereby creating a contiguous fusion of two polypeptides. In some embodiments, a grafted nucleic acid encodes a chimeric protein, such that one polypeptide is effectively appended to another polypeptide (e.g. directly conjugated before the N-terminus or after the C-terminus), thereby creating a contiguous fusion of two polypeptides. In some embodiments, the term grafting refers to joining or uniting of at least two polypeptides, or fragments thereof, such that one of the at least two polypeptides or fragments thereof is inserted within another of the at least two polypeptides or fragments thereof. In some embodiments, the term grafting refers to joining or uniting of at least two polypeptides or fragments thereof such that one of the at least two polypeptides or fragments thereof is appended to another of the at least two polypeptides or fragments thereof.

In some embodiments, the disclosure relates to an adeno-associated virus (AAV) capsid protein that is conjugated to one or more bone-targeting moieties. A “bone-targeting moiety” generally refers to a small molecule, peptide, nucleic acid, etc., that facilitates trafficking of an rAAV to bone or bone tissue. For example, in some embodiments, a bone-targeting moiety is a peptide or small molecule that binds to a receptor on a bone cell (e.g., OB, OC, osteocyte, etc.). Examples of bone-targeting moieties include but are not limited to alendronate (ALE), polypeptides such as cyclic arginine-glycine-aspartic acid-tyrosine-lysine (cRGDyk; SEQ ID NO: 40), Asp-Asp-Asp-Asp-Asp-Asp-Asp-Asp (D-Asp8; SEQ ID NO: 41), and aptamers such as CH6. A bone-targeting moiety may be conjugated directly to a capsid protein or conjugated to a capsid protein via a linker molecule (e.g., an amino acid linker, a PEG linker, etc.).

In some embodiments, a linker is a glycine-rich linker. In some embodiments, a linker comprises at least two glycine residues. In some embodiments, a linker comprises GGGGS (SEQ ID NO: 13). In some embodiments, the linker comprises a formula selected from the group consisting of: [G]ₙ (SEQ ID NO: 14), [G].S (SEQ ID NO: 15), [GS]. (SEQ ID NO: 16), and [GGSG]ₙ (SEQ ID NO: 17), for example, wherein G is glycine and wherein n is an integer greater than one (e.g., 2, 3, 4, 5, 6, 7, 8, 9, 10, 11, 12, 13, 14, 15, 16, 17, 18, 19, 20, 21, 22, 23, 24, 25 or more). In some embodiments, n is an integer in a range of 2 to 10, 2 to 20, 5 to 10, 5 to 15, or 5 to 25. Accordingly, in some embodiments, a heterologous targeting peptide is conjugated to a linker.

In some embodiments, a capsid protein comprises one or more azide-bearing unnatural amino acids which are capable of reacting with an ADIBO-tagged bone-targeting moiety (e.g., via “click chemistry” to form a capsid protein-bone-targeting moiety conjugate. Capsid proteins comprising unnatural azide-bearing amino acids are described, for example by Zhang et al. (2016) Biomaterials 80:134-145, and use of ADIBO-based click chemistry for peptide conjugation is described, for example by Prim et al. (2013) Molecules 18(8):9833-49.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. In some embodiments, a host cell is a bacterial cell, yeast cell, insect cell (Sf9), or a mammalian (e.g., human, rodent, non-human primate, etc.) cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

In some embodiments, the rAAV may comprise at least one modification which increases targeting of the rAAV to bone cells (e.g., osteoblasts, osteoclasts, osteocytes, chondrocytes). Non-limiting examples of modifications which increase targeting of the rAAV to bone cells include heterologous bone-targeting peptides, and AAV capsid serotypes (e.g., AAV1, AAV4, AAV5, AAV6, AAV6.2, AAV7, AAV8, AAV9, AAV10, AAVrh39, AAVrh43).

Expression of SHN3 in a cell or subject may be decreased by between 50% and 99% (e.g., any integer between 50% and 99%, inclusive) compared to a control using rAAVs of the present disclosure. Expression of SHN3 in a cell or subject may be decreased by between 75% and 90% compared to a control using rAAVs of the present disclosure. Expression of SHN3 in a cell or subject may be decreased by between 80% and 99% compared to a control using rAAVs of the present disclosure.

Expression of SOST in a cell or subject may be decreased by between 50% and 99% compared to a control (e.g., any integer between 50% and 99%, inclusive) using rAAVs of the present disclosure. Expression of SOST in a cell or subject may be decreased by between 75% and 90% compared to a control using rAAVs of the present disclosure. Expression of SOST in a cell or subject may be decreased by between 80% and 99% compared to a control using rAAVs of the present disclosure.

The foregoing methods for packaging recombinant vectors in desired AAV capsids to produce the rAAVs of the disclosure are not meant to be limiting and other suitable methods will be apparent to the skilled artisan.

### Bone Graft Substitute

The present disclosure provides a bone graft substitute for implantation to a subject in need thereof. The bone graft substitute is capable of aiding or accelerating the healing of bone fracture or bone defects, especially defects that are caused by critical-size bone defects. The healing of critical-sized skeletal defects (CSD) remains one of the most challenging problems in orthopedic management because CSDs are unable to heal without interventions such as insertion of a bone graft.

In some embodiments, a recombinant adeno-associated virus (rAAV) as disclosed herein is attached to the bone graft substitute. In some embodiments, the rAAV comprises capsids that target bones. For example, AAV9 capsid proteins can be used. In some embodiments, a substrate as disclosed herein is attached to the bone graft substitute. In some embodiments, the substrate is a solid substrate. As used herein, a “solid substrate” can be any naturally occurring or artificial matters or materials that are not in an aqueous or liquid form and can be useful for stabilizing the bone graft substitute for homing to the areas of bone defects or fracture. In some embodiments, the substrate is hydroxyapatite. In some embodiments, the hydroxyapatite is a hydroxyapatite (HA)-based scaffold.

In some embodiments, the HA-attached bone graft substitute increases the delivery efficiency of the rAAV by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to the same bone graft substitute without the attachment of the HA.

In some embodiments, peptide motifs are attached to the rAAVs as disclosed herein. In some embodiments, the peptide motifs as disclosed herein are short stretches of amino acid sequences that are homing to OC-enriched bone-forming surfaces. In some embodiments, the peptide motifs is (Asp-Ser-Ser)₆ (SEQ ID NO: 11). In some embodiments, the peptide motifs as disclosed herein are short stretches of amino acid sequences that are OC-enriched bone-resorbing surfaces. In some embodiments, the peptide motifs is (Asp)₁₄.

In some embodiments, the HA and peptide motif attached bone graft substitute as disclosed herein increases the genome copies of the rAAV by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to the same bone graft substitute without the attachment of the HA and the protein motif.

In some embodiments, the bone graft substitute is integrated into the bone tissues. In some embodiments, the bone tissues comprise tissues in the area that have bone fracture or defect. In some embodiments, the bone tissues comprise CSD injury sites. In some embodiments, the bone tissues comprise skeletal tissues.

In some embodiments, the bone graft substitute is an autologous bone graft. In some embodiments, the bone graft substitute comprises an allogeneic bone graft. In some embodiments, the bone graft substitute comprises a decellularized bone matrix. In some embodiments, the bone graft substitute comprises a hydroxyapatite scaffold. In some embodiments, the bone graft substitute comprises a calcium-phosphate scaffold. In some embodiments, the bone graft substitute comprises a skeletal bone organoid obtained from MSCs.

In some embodiments, the bone graft substitute is incubated ex vivo with human bone marrow-derived stromal cells prior to implantation to the subject. In some embodiments, the bone graft substitute is incubated ex vivo with the rAAV prior to implantation to the subject. In some embodiments, the bone graft substitute is incubated ex vivo with a composition comprising cells of osteoblastic lineage prior to implantation to the subject.

In some embodiments, the bone graft substitute such as allogenic bone is incubated ex vivo in a reagent. In some embodiments, the reagent is sterile PBS. In some embodiments, the bone graft substitute is incubated in the reagent with the rAAV as disclosed herein. In some embodiments, the reagent is any reagent that is suitable for the production of the bone graft substitute. In some embodiments, the incubation period can last for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 minutes. In some embodiments, the incubation period can last for any period of time depending on the bone graft substitute being made (e.g., the size and source of the bone).

In some embodiments, the incubation can be conducted at room temperature. In some embodiments, the incubation can be conducted under any temperature that are suitable for the production of the bone graft substitute as disclosed herein.

### Modes of Administration and Compositions

The rAAVs of the disclosure may be delivered to a subject in compositions according to any appropriate methods known in the art. For example, an rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

In some embodiments, the rAAV comprises an amiR-33 targeting SHN3 and/or SOST having the sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to any one of SEQ ID NOs: 3-10. In some embodiments, the rAAV comprises an amiR-33 targeting SHN3 and/or SOST having the sequence set forth in any one of SEQ ID NO: 3-10 (or the complementary sequence thereof), or a portion thereof. In some embodiments, a composition further comprises a pharmaceutically acceptable carrier.

In some embodiments, the capsid protein further comprises a heterologous bone-targeting peptide, for example a DSS-AAV9 capsid protein.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the bone (e.g., bone tissue) of a subject. By “bone tissue” is meant all cells and tissue of the bone and/or joint (e.g., cartilage, axial and appendicular bone, etc.) of a vertebrate. Thus, the term includes, but is not limited to, osteoblasts, osteocytes, osteoclasts, chondrocytes, and the like. Recombinant AAVs may be delivered directly to the bone by injection into, e.g., directly into the bone, via intrasynovial injection, knee injection, femoral intramedullary injection, etc., with a needle, catheter or related device, using surgical techniques known in the art. In some embodiments, rAAV as described in the disclosure are administered by intravenous injection. In some embodiments, the rAAV are administered by intramuscular injection.

Aspects of the instant disclosure relate to compositions comprising a recombinant AAV comprising a capsid protein and a nucleic acid encoding a transgene, wherein the transgene comprises a nucleic acid sequence encoding one or more bone metabolism modulating agents. In some embodiments, the nucleic acid further comprises one or more AAV ITRs. In some embodiments, a composition further comprises a pharmaceutically acceptable carrier.

In some embodiments, compositions comprise a recombinant AAV comprising a capsid protein and a nucleic acid comprising a first region encoding an AAV ITR and a second region comprising a transgene, wherein the transgene encodes an artificial microRNA that targets SHN3 or SOST.

Aspects of the disclosure provide a method of decreasing SHN3 and/or SOST expression in a cell. A cell may be a single cell or a population of cells (e.g., culture). A cell may be in vivo (e.g., in a subject) or in vitro (e.g., in culture). A subject may be a mammal, optionally a human, a mouse, a rat, a non-human primate, a pig, a dog, a cat, a chicken, or a cow.

Expression of SHN3 and/or SOST in a cell or subject may be decreased by between 50% and 99% (e.g., any integer between 50% and 99%, inclusive) using isolated nucleic acids, rAAVs, or compositions of the present disclosure. Expression of SHN3 and/or SOST in a cell or subject may be decreased by between 75% and 90% using isolated nucleic acids, rAAVs, or compositions of the present disclosure. Expression of SHN3 and/or SOST in a cell or subject may be decreased by between 80% and 99% using isolated nucleic acids, rAAVs, or compositions of the present disclosure.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs or the rAAV-based therapeutic constructs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

In some embodiments, the rAAVs or the rAAV-based therapeutic constructs as disclosed herein are administered locally. In some embodiments, the rAAVs or the rAAV-based therapeutic constructs as disclosed herein are administered directly to the areas of bone injuries or defects by implanting the bone grafts that are attached to the rAAVs. In some embodiments, the areas of bone injuries or defects include gap sites and nonunion of bone fractures that are caused by bone injuries or defects such as critical-sized bone defects. In some embodiments, the rAAVs or the rAAV-based therapeutic constructs that are locally delivered to the areas of bone injuries or defects are grafted with a bone-targeting peptide motif, ((AspSerSer)₆, DSS). In some embodiments, the rAAVs or the rAAV-based therapeutic constructs that are locally delivered to the areas of bone injuries or defects are attached with hydroxyapatite (HA). In some embodiments, the bone grafts that are delivered locally and implanted to the areas of bone injuries or defects are allogenous bone grafts. In some embodiments, the bone grafts that are delivered locally and implanted to the areas of bone injuries or defects are autogenous bone grafts.

In certain circumstances, it will be desirable to deliver rAAVs or rAAV-based therapeutic constructs in suitably formulated pharmaceutical compositions disclosed herein either subcutaneously, intraopancreatically, intranasally, parenterally, intravenously, intramuscularly, intrathecally, femoral intramedullary, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection. In some embodiments, a preferred mode of administration is by local administration such as implantation of bone grafts as disclosed herein.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An “effective amount” of an rAAV is an amount sufficient to target infect an animal, target a desired tissue (e.g., bone tissue). The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In certain embodiments, 10¹² or 10¹³ rAAV genome copies is effective to target bone tissue.

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.) Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 pm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 pm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Therapeutic Methods

Methods for delivering an effective amount of a transgene (e.g., an isolated nucleic acid or rAAV encoding a WNT antagonist such as SOST and SHN3) to a subject are provided by the disclosure. In some embodiments, the methods comprise the step of administering to a subject an effective amount of an isolated nucleic acid encoding an interfering RNA capable of inhibiting bone loss (e.g., bone loss due to bone fracture, osteoporosis). In some embodiments, the methods comprise the step of administering to a subject an effective amount of an isolated nucleic acid encoding an interfering RNA capable of reversing bone loss. Thus, in some embodiments, isolated nucleic acids, rAAVs, and compositions described herein are useful for treating a subject having or suspected of having a disease or disorder associated with bone loss.

As used herein, a “disease or disorder associated with dysregulated bone metabolism” refers to a condition characterized by an imbalance between bone deposition and bone resorption resulting in either 1) abnormally increased bone deposition (e.g., formation) relative to a healthy individual (e.g., a subject not having a disease characterized by imbalance between bone deposition and bone resorption), or 2) abnormally decreased bone deposition (e.g., formation) relative to a healthy individual (e.g., a subject not having a disease characterized by an imbalance between bone deposition and bone resorption), or 3) abnormally increased bone resorption (e.g., breakdown) relative to a healthy individual (e.g., a subject not having a disease characterized by imbalance between bone deposition and bone resorption), or 4) abnormally decreased bone resorption (e.g., breakdown) relative to a healthy individual (e.g., a subject not having a disease characterized by imbalance between bone deposition and bone resorption). A “disease associated with reduced bone density” refers to a condition characterized by increased bone porosity resulting from either 1) abnormally decreased bone deposition (e.g., formation) relative to a healthy individual (e.g., a subject not having a disease characterized by decreased bone density), or 2) abnormally increased bone resorption (e.g., breakdown) relative to a healthy individual (e.g., a subject not having a disease characterized by decreased bone density). A disease associated with increased bone porosity may arise from either 1) abnormally decreased OB and/or osteocyte differentiation, function, or activity relative to a healthy individual (e.g., a subject not having a disease characterized by decreased bone density) and/or 2) abnormally increased OC differentiation, function, or activity relative to a healthy individual (e.g., a subject not having a disease characterized by decreased bone density). “Porosity” generally refers to the volume of fraction of bone not occupied by bone tissue.

A “disease associated with increased bone density” refers to a condition characterized by decreased bone porosity resulting from either 1) abnormally increased bone deposition (e.g., formation) relative to a healthy individual (e.g., a subject not having a disease characterized by increased bone density), or 2) abnormally decreased bone resorption (e.g., breakdown) relative to a healthy individual (e.g., a subject not having a disease characterized by increased bone density). A disease associated with decreased bone porosity may arise from either 1) abnormally increased OB and/or osteocyte differentiation, function, or activity relative to a healthy individual (e.g., a subject not having a disease characterized by increased bone density) and/or 2) abnormally decreased OC differentiation, function, or activity relative to a healthy individual (e.g., a subject not having a disease characterized by increased bone density). Aspects of the present disclosure provide methods of treating a disease or disorder associated with dysregulated bone metabolism. Dysregulated bone metabolism may be diseases associated with reduced bone density (e.g., osteoporosis, critical sized-bone defects, a mechanical disorder resulting from disuse or injury). Dysregulated bone metabolism may be diseases associated with increased bone density (e.g., osteopetrosis, pycnodysostosis, sclerosteosis, acromegaly, fluorosis, myelofibrosis, hepatitis C-associated osteosclerosis, heterotrophic ossification). In some embodiments, a subject having a disease or disorder associated with dysregulated bone metabolism has one or more signs or symptoms of an inflammatory disease. Examples of inflammatory diseases include but are not limited to rheumatoid arthritis (RA), psoriasis, ankylosing spondylitis, systemic lupus erythematosus, multiple sclerosis, inflammatory bowel diseases, periodontitis, and pemphigus vulgaris. In some embodiments, a subject having an inflammatory disease is characterized as having an increased level or amount of inflammatory cytokines (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF), etc.) or other markers of inflammation, relative to a normal, healthy subject. In some embodiment, the subject having an inflammatory disease has the level or amount of inflammatory cytokines (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF), etc.) or other markers of inflammation increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a normal, healthy subject.

In some embodiments, administering the isolated nucleic acid, the rAAV, the vector results in an increase of various biomarkers associated with the Wnt signaling pathway. In some embodiments, administering the isolated nucleic acid, the rAAV, the vector results in an increase of receptor activator of nuclear factor kappa-B ligand (RANKL) by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering the isolated nucleic acid, the rAAV, the vector, the bone graft substitute results in an increase of osteoprotegerin (OPG) by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering the isolated nucleic acid, the rAAV, the vector, the bone graft substitute results in an increase of Axin2 by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering the isolated nucleic acid, the rAAV, the vector, the bone graft substitute results in an increase of Lef1 by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering the isolated nucleic acid, the rAAV, the vector, the bone graft substitute improves bone formation and/or bone healing in a subject by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering the isolated nucleic acid, the rAAV, the vector, the bone graft substitute stimulate bone regeneration and/or reversing bone loss in a subject by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

As used herein, the improvement or stimulation is relative to a control. The control can be in a state that is prior to the administration of the isolated nucleic acid, the rAAV, the vector, and the bone graft substitute. The improvement or stimulation is relative to a subject that has not been administered the isolated nucleic acid, the rAAV, the vector, and the bone graft substitute.

As used herein, a “normal, healthy subject” refers to a subject who does not have, is not suspected of, or is at risk of developing a disease or disorder. In some embodiments, the disease or disorder is an inflammatory disease. In some embodiments, the disease or disorder is associated with bone metabolism. In some embodiments, a normal, healthy subject can be a control described herein.

As used herein, the term “treating” refers to the application or administration of a composition, isolated nucleic acid, vector, or rAAV as described herein to a subject having bone loss or a predisposition toward a bone loss condition, with the purpose to cure, heal, alleviate, relieve, alter, remedy, ameliorate, improve, or affect the disorder, the symptom of the disease, or the predisposition toward the inflammatory condition.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of inflammatory diseases includes initial onset and/or recurrence.

In some embodiments, methods of treating a disease or disorder associated with bone fracture and critical-sized bone defect or osteoporosis comprise administering to a subject in need thereof a recombinant AAV (rAAV) comprising a transgene. A rAAV may comprise a modification that promotes its targeting to bone cells (e.g., osteoclasts and osteoblasts). Non-limiting modifications of rAAVs that promote its targeting to bone cells include modification of capsid proteins with heterologous bone-targeting peptides, modification of rAAV vectors with bone-specific promoters, and use of AAV serotypes with increased targeting to bone relative to other tissues.

In some embodiments, the rAAV comprising the heterologous bone-targeting peptide comprises a transgene which upregulates or downregulates a target gene associated with dysregulation of bone metabolism. In some embodiments, the transgene upregulates the expression of a target gene that is decreased in a disorder associated with reduced bone density (e.g., osteoporosis, critical sized-bone defects, a mechanical disorder resulting from disuse or injury). In some embodiments, the transgene downregulates the expression of a target gene that is increased in a disorder associated with reduced bone density. In some embodiments, the transgene upregulates the expression of a target gene that is decreased in a disorder associated with reduced bone density (e.g., osteoporosis, critical sized-bone defects, a mechanical disorder resulting from disuse or injury). In some embodiments, the transgene downregulates the expression of a target gene that is increased in a disorder associated with reduced bone density. Aspects of the disclosure provide methods for treating a disease or disorder associated with a disease of disorder characterized by dysregulation of bone metabolism comprising administering to a subject a rAAV comprising a capsid protein and an isolated nucleic acid encoding an inhibitory nucleic acid. The rAAV may comprise an inhibitory nucleic acid (e.g., siRNA, shRNA, miRNA, or ami-RNA). The inhibitory nucleic acid may decrease or increase expression of a target gene associated with a disease or disorder characterized by dysregulation of bone metabolism.

In some embodiments, the present disclosure provides a method of treating disease or disorder associated with osteoporosis, critical sized-bone defects, and bone fractures. The method comprises administering to a subject in need thereof a rAAV or an isolated nucleic acid comprising a transgene that targets a gene associated with the Wnt signaling pathway. In some embodiments, the rAAV or isolated nucleic acid comprises a transgene encoding an artificial microRNA that targets a gene associated with reduced bone density. In some embodiments, the target gene is SHN3 or SOST.

As disclosed herein, “identity” of sequences refers to the measurement or calculation of the percent of identical matches between two or more sequences with gap alignments addressed by a mathematical model, algorithm, or computer program that is known to one of ordinary skill in the art. The percent identity of two sequences (e.g., nucleic acid or amino acid sequences) may, for example, be determined using Basic Local Alignment Search Tool (BLAST®) such as NBLAST® and XBLAST® programs (version 2.0). Alignment technique such as Clustal Omega may be used for multiple sequence alignments. Other algorithms or alignment methods may include but are not limited to the Smith-Waterman algorithm, the Needleman-Wunsch algorithm, or Fast Optimal Global Sequence Alignment Algorithm (FOGSAA).

Expression of the target gene (e.g., SHN3, SOST etc.) in a cell or subject may be decreased by between 50% and 99% (e.g., any integer between 50% and 99%, inclusive) using methods of the present disclosure. Expression of the target gene (e.g., SHN3, SOST, etc.) in a cell or subject may be decreased by between 75% and 90% using methods of the present disclosure. Expression of SHN3 in a cell or subject may be decreased by between 80% and 99% using methods of the present disclosure. Expression of SOST in a cell or subject may be decreased by between 80% and 99% using methods of the present disclosure.

In some embodiments, an “effective amount” or “amount effective of a substance in the context of a composition or dose for administration to a subject refers to an amount sufficient to produce one or more desired effects (e.g., to preserve bone tissue or reverse bone loss). In some embodiments, an effective amount of an isolated nucleic acid is an amount sufficient to transfect (or infect in the context of rAAV-mediated delivery) a sufficient number of target cells of a target tissue of a subject. In some embodiments, a target tissue is bone tissue (e.g., bone and bone tissue cells, such as OBs, OCs, osteocytes, chondrocytes, etc.). In some embodiments, an effective amount of an isolated nucleic acid (e.g., which may be delivered via an rAAV) may be an amount sufficient to have a therapeutic benefit in a subject, e.g., to increase activity or function of OBs and/or osteocytes, to inhibit activity of OBs and/or osteocytes, to increase activity of function of OCs, to inhibit activity or function of OCs, etc. In some embodiments, an effective amount of an isolated nucleic acid disclosed herein may partially or fully rescue bone losses. In some embodiments, an effective amount of an isolated nucleic acid disclosed herein may partially or fully alleviate the effects of the genes that cause bone losses. An effective amount can also involve delaying the occurrence of an undesired response. The effective amount will depend on a variety of factors such as, for example, the species, age, weight, health of the subject, the severity of a condition, the tissue to be targeted, the specific route of administration and like factors, and may thus vary among subject and tissue as described elsewhere in the disclosure.

Exemplary embodiments of the invention will be described in more detail by the following examples. These embodiments are exemplary of the invention, which one skilled in the art will recognize is not limited to the exemplary embodiments.

### EXAMPLES

### Example 1. Experimental Designs and Protocols

### Methods

Cell lines, plasmids, and antibodies. Ocy454 cells were obtained from Massachusetts General Hospital (MGH, Boston, MA) and maintained in a-MEM medium (Corning) supplemented with 10% FBS (Corning) and 1% penicillin/streptomycin (Corning) at 33° C. with 5% CO₂. For osteocyte differentiation, cells were transferred to 37° C. when they were confluent at 33° C. and cultured for 6-12 days for the analysis of osteocyte gene expression. HEK293T cells or C2H10T1/2 cells were purchased from ATCC and grown in DMEM (Corning) supplemented with 10% FBS (Corning), 2 mM L-glutamine (Corning), 1% nonessential amino acids (Corning), and 1% penicillin/streptomycin (Corning). Full length or truncated mutants of murine Shn3 cDNAs were PCR-amplified and cloned into pEF-Nuc mammalian expression vector (Invitrogen) or pHASE/PGK-PURO lentiviral vector. Human SHN3 shRNA sequence (CCGGGCCTTGAACTTACCATGGAAACTCGAGTTTCCATGGTAAGTTCAAGGCTTTTT ; SEQ ID NO: 18) was cloned into the pLK0.1 lentiviral vector). Antibodies specific to Flag (Sigma, F1804), HSP90α/β (Biolegend, 675402), GAPDH (EMD Millipore, CB1001), and GFP (Takara, 632381) were used. Hydroxyapatite-based scaffold was kindly gifted from Osteogene 30 Tech. Inc.

rAAV Vector Design and Production

Bone-targeting AAV9 (rAAV9.DSS-Nter, rAAV9.D14-Nter) vectors were generated as described in previous studies. DNA sequences for amiR-33-ctrl, amiR-sost, amiR-shn3, hs-amiR-shn3, and amiR-sost/shn3 were synthesized as gBlocks, cloned into the intronic region of the pAAVsc-CB6-Egfp plasmid at the restriction enzyme sites (PstI and BglII), and packaged into AAV9.DSS-Nter capsid. rAAV production was performed by transient transfection of HEK293 cells, purified by CsCl sedimentation, titered by droplet digital PCR (ddPCR) on a QX200 ddPCR system (Bio-Rad) using the Egfp prime/probe set as previously described3. The sequences of gBlocks and oligonucleotides for ddPCR and are listed in Table 2.

### Mice

Shn3^{fl/fl} mice(48) were generated as previously described, crossed with Prxl-cre mice (Jackson laboratory) and maintained on C57BL/6J background. Wildtype C57BL/6J and SCID mice were purchased from the Jackson laboratory. Mouse genotypes were determined by PCR on tail genomic DNA; primer sequences are available upon request. All animals were used in accordance with the NIH Guide for the Care and Use of Laboratory Animals and were handled according to protocols approved by the University of Massachusetts Medical School on animal care (IACUC).

### MicroCT Analysis

MicroCT was used for qualitative and quantitative assessment of trabecular and cortical bone microarchitecture and performed by an investigator blinded to the genotypes of the animals under analysis. Femurs excised from the indicated mice were fixed with 10% neutral buffered formalin and scanned using a microCT 35 (Scanco Medical) with a spatial resolution of 7 μm.

For trabecular bone analysis of the distal femur, an upper 2.1 mm region beginning 280 m proximal to the growth plate was contoured. For cortical bone analysis of femur, a midshaft region of 0.6 mm in length was used. MicroCT scans of L4 spinal segments were performed using isotropic voxel sizes of 12 μm. 3D reconstruction images were obtained from contoured 2D images by methods based on distance transformation of the binarized images. Alternatively, the Inveon multimodality 3D visualization program was used to generate fused 3D viewing of multiple static or dynamic volumes of microCT modalities (Siemens Medical Solutions USA, Inc). All images presented are representative of the respective genotypes (n>5).

### Histology, Histomorphometry, and Immunofluorescence

For histological analysis, femurs and vertebrae were dissected from the mice treated with rAAVs vectors, fixed in 10% neutral buffered formalin for two days, and decalcified by 5% tetrasodium EDTA for 2-4 weeks. Tissues were dehydrated by passage through an ethanol series, cleared twice in xylene, embedded in paraffin, and sectioned at a thickness of 6 μm along the coronal plate from anterior to posterior. Decalcified femoral sections were stained with hematoxylin and eosin (H&E) or tartrate-resistant acid phosphatase (TRAP).

For dynamic histomorphometric analysis, 25 mg/kg calcein (Sigma, C0875) and 50 mg/kg alizarin-3-methyliminodiacetic acid (Sigma, A3882) dissolved in 2% sodium bicarbonate solution were subcutaneously injected into mice at six day-interval. After fixed in 10% neutral buffered formalin for two days, undecalcified femur samples were embedded in methylmethacrylate and proximal metaphysis is sectioned longitudinally (5 m) and stained with McNeal's trichrome for osteoid assessment, toluidine blue for osteoblasts, and TRAP for osteoclasts. A region of interest is defined and bone formation rate/bone surface (BFR/BS), mineral apposition rate (MAR), bone surface (BS), osteoblast surface (Ob.S/BS), and osteoclast surface (Oc.S/BS) are measured using a Nikon Optiphot 2 microscope interfaced to a semiautomatic analysis system (Osteometrics). Measurements were taken on two sections/sample (separated by ˜25 m) and summed prior to normalization to obtain a single measure/sample in accordance with ASBMR standards. This methodology has undergone extensive quality control and validation and the results were assessed by two different researchers in a blinded fashion.

### Luciferase Reporter Assay

AAV-treated Ocy454 osteocyte line was transfected with β-catenin-responsive reporter gene (TopFlash-luc) using the Effectene transfection reagent (Qiagen) and cultured for 6 days in the presence of recombinant WNT3a (25 μg/ml, R&D systems). Luciferase assay was performed according to the manufacturer's protocol (Promega).

### Quantitative RT-PCR Analysis

Total RNA was purified from cells using QIAzol (QIAGEN) and cDNA was synthesized using the High-Capacity cDNA Reverse Transcription Kit from Applied Biosystems. Quantitative RT-PCR was performed using SYBR® Green PCR Master Mix (Bio-Rad) with CFX connect RT-PCR detection system (Bio-Rad). To measure mRNA levels in bone tissues, after removal of bone marrow, tibias were snap-frozen in liquid nitrogen for 30 sec and in turn homogenized in 1 ml of QIAzol for 1 min. Primers used for PCR are described in Table 2.

### Immunoblotting Analysis

Cells were lysed in TNT lysis buffer (50 mM Tris-HCl (pH 7.4), 150 mM NaCl, 1% Triton X-100, 1 mM EDTA, 1 mM EGTA, 50 mM NaF, 1 mM Na₃VO₄, 1 mM PMSF and protease inhibitor cocktail (Sigma)) and protein amounts from cell lysates were measured using DC protein assay (Bio-Rad). Equivalent amounts of proteins were subjected to SDS-PAGE, transferred to Immunobilon-P membranes (Millipore), immunoblotted with the indicated antibodies, and developed with ECL (Thermo fisher scientific). Immunoblotting with anti-HSP90 antibody was used as a loading control.

Effects of rAAV9-Mediated Delivery of WNT-Modulating Gene Silencers on Bone Formation

200 μl of rAAV9.DSS carrying amiR-ctrl, amiR-sost, amiR-shn3, hs-amiR-shn3, or amiR-sost/shn3 (5×10¹³ vg/kg) was intravenously (i.v.) injected into mice and two months later, mice were subcutaneously injected with calcein and alizarin-3-methyliminodiacetic acid at six day-interval for dynamic histomorphometric analysis. Non-labeled mice were used to monitor EGFP expression using the IVIS-100 optical imaging or cryo-sections.

### Therapeutic Effects of Systemically Delivered AAV Vectors in Mouse Models of Osteoporosis

Mouse models of postmenopausal osteoporosis were generated by anesthetizing and bilaterally ovariectomizing (OVX) three-month-old female mice (Jackson Laboratory). 6 weeks after the surgery, sham or OVX mice were i.v. injected with 200 μl of rAAV9.DSS carrying rAAV9.DSS carrying amiR-ctrl, amiR-sost, amiR-shn3, or amiR-sost/shn3 (5×10¹³ vg/kg). Mice were randomly divided into five groups (sham+rAAV9.DSS-amiR-ctrl, OVX+rAAV9-amiR-ctrl, OVX+rAAV9-amiR-shn3, OVX+rAAV9.DSS.amiR-sost, OVX+rAAV9.DSS.amiR-sost/shn3). 8 weeks after the injection, mice were subcutaneously injected with calcein and alizarin-3-methyliminodiacetic acid at six day-intervals for dynamic histomorphometric analysis. Non-labeled mice were used to monitor EGFP expression using the IVIS-100 optical imaging or frozen-sections. As a mouse model of senile osteoporosis, 18-month-old male mice were i.v. injected with 200 μl of rAAV9.DSS vectors (5×10¹³ vg/kg) and 2 months later, skeletal analyses were performed using microCT, histology, and dynamic histomorphometric analysis.

### Effects of Systemically Delivered AAV Vectors on Bone Regeneration in Uni-Cortical Bone Defect

200 μl of rAAV9.DSS carrying amiR-ctrl, amiR-shn3, amiR-sost, or amiR-sost/shn3 (5×10¹³ vg/kg) was i.v. injected 2 weeks before the surgery. The uni-cortical bone defect was conducted under general anesthesia (isoflurane, 1˜4%) and the pain was controlled by subcutaneous injection of buprenorphine (0.03 mg/kg) 1 hour before surgery. 2-month-old female mice were placed in the lateral decubitus position, and the sterile environment of a surgical site was maintained with disinfection and surgical cloth. A 1.0-cm skin incision was made on the lateral aspect of the femur, and the femur was exposed by accessing through the vastus lateralis. A bone defect with a length of 3-mm and a width of 1-mm was made using a 1-mm-sized motorized burr while protecting the posterior femoral nerve. The defect site was irrigated with PBS to remove a bone fragment within the medulla, and the fascia and skin were closed with the Vicryl 4/0 and Nylon 5/0. 2 weeks after the surgery, fluorescence microscopy and qPCR analysis in the tibial bone RNA were performed to assess AAV's transduction and knockdown efficiency, respectively. Skeletal analyses were performed using microCT and histology.

### Effects of Systemically Delivered AAV Vectors on Bone Fracture Healing

200 μl of rAAV9.DSS carrying amiR-ctrl, amiR-shn3, amiR-sost, or amiR-sost/shn3 (5×10¹³ vg/kg) was i.v. injected 2 weeks before the surgery. 12-week-old male mice were placed in the lateral recumbency and covered with a sterile surgical drape. A longitudinal skin incision was made along the lateral aspect of the thigh from the stifle joint to the hip. The lateral aspect of the femur was exposed by parting the vastus lateralis muscle and the rectus femoris muscle to expose the length of the femur while preserving the femoral nerve. The middle of the femoral shaft was excised with a surgical saw. Intramedullary fixation was performed with a 25G needle penetrating from the patella furrow of the distal femur to the greater trochanter tip of the femur. Both ends of the needle were bent and then cut with a wire cutter, leaving 1 mm. The fascia was sutured using a 4/0 Vicryl suture, and then the skin was closed using a 4/0 Nylon suture. X-radiography of the injured legs was performed to monitor fracture healing 2 weeks post-surgery. 4 weeks later, fluorescence microscopy and qPCR analysis in the tibial bone RNA were performed to assess AAV's transduction and knockdown efficiency, respectively. microCT and histology were performed for skeletal analyses.

### Effects of Locally Delivered AAV Vectors on Critical-Sized Bone Defect

Allogenous femoral bone graft was prepared by decellularization using sonication and stored in −80° C. It was thawed in cold PBS before the surgery begins. 3-month-old male mice were placed in the lateral recumbency and covered with a sterile surgical drape. A longitudinal 2.0 cm skin incision was made along the lateral aspect of the thigh from the stifle joint to the hip. The shaft of the femur was exposed by dissecting the muscle fascia slightly anterior to lateral intermuscular septum while protecting neurovascular bundle located posteriorly. To make the bone defect artificially, ostectomy of the femur (4 mm length) was conducted with the oscillating saw. Then the allogenous femoral bone graft was inserted into the gap site, and the 23G needle was passed through the medulla of femoral bone and allogenous bone to fix entire surgical structures. After irrigating the operation site with PBS to remove a bone fragment from the muscle or other soft tissue, the fascia and skin were closed with the Vicryl 4/0 and Nylon 5/0. 12 weeks after the surgery, skeletal analyses were performed using microCT and histology.

TABLE 1
Summary of the effects of the single and dual AAV silencers on bone modulation

| | | | | SHN3/SOST- |
|---|---|---|---|---|
| | WT | SHN3-silencer | SOST-silencer | dual silencer |
| WNT signaling | weak | intermediate | intermediate | strong |
| Osteoblast | normal | Increase | Increase | Increase and |
| | | | | wane over time |
| Osteoclast | normal | No effect | mild decrease | Ablation by OPG |
| Bone accrual | normal | Slow and mild | Slow and mild | Fast and strong |
| | | increase | increase | increase |
| Bone regeneration | normal | Increase | Increase | Increase |
| Osteoporosis | Low bone | Complete reversal | Partial reversal | Complete reversal |
| | mass | | | |
| Bone fracture | normal | Increase | Increase | No effect |
| healing | | | | |
| Critical sized bone | normal | Increase | Increase | No effect |
| defect healing | | | | |

### Statistics and Reproducibility

All experiments were carried out at least two or three times, for IHC, histological staining, and immunoblotting, representative images are shown. All data are shown as the mean±Standard Deviation (SD). We first performed the Shapiro-Wilk normality test for checking normal distributions of the groups. If normality tests passed, two-tailed, unpaired Student's t-test and if normality tests failed, and Mann-Whitney tests were used for the comparisons between two groups. For the comparisons of three or four groups, we used one-way ANOVA if normality tests passed, followed by Tukey's multiple comparison test for all pairs of groups. The GraphPad PRISM software (ver.9.0.0, La Jolla, CA) was used for statistical analysis. P<0.05 was considered statistically significant.

### Example 2. Development of Bone-Targeting AAV Gene Silencers that Control WNT Signaling

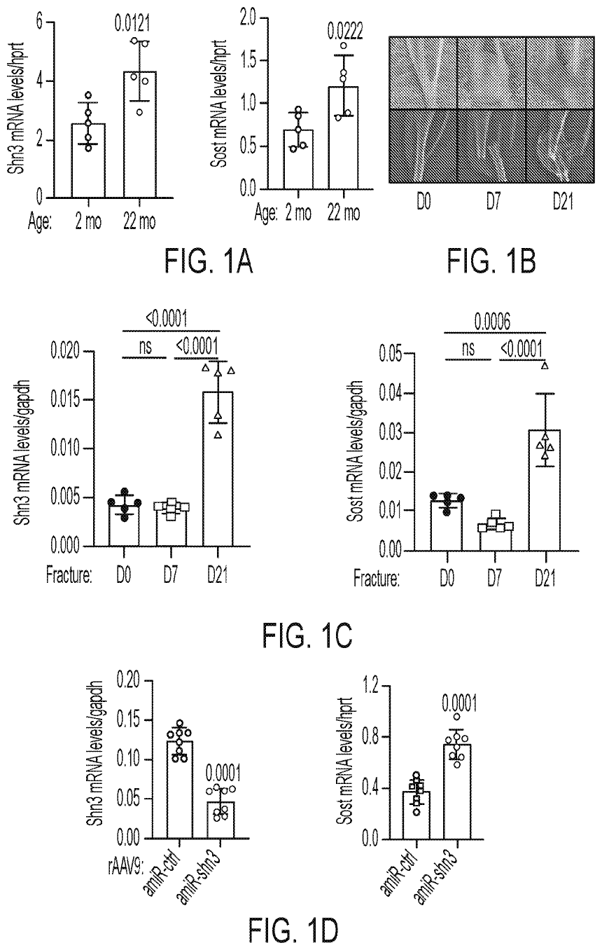

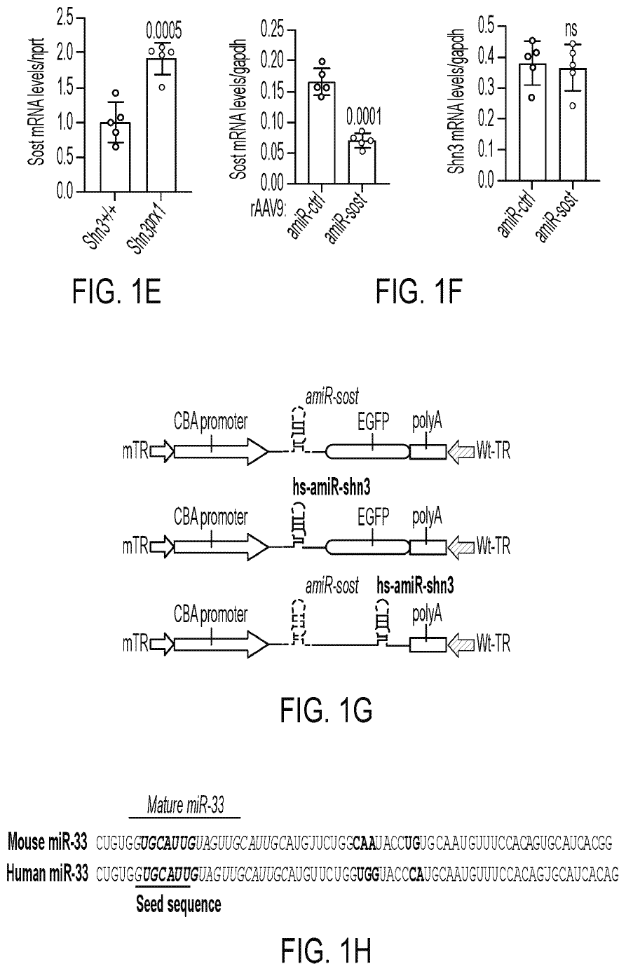

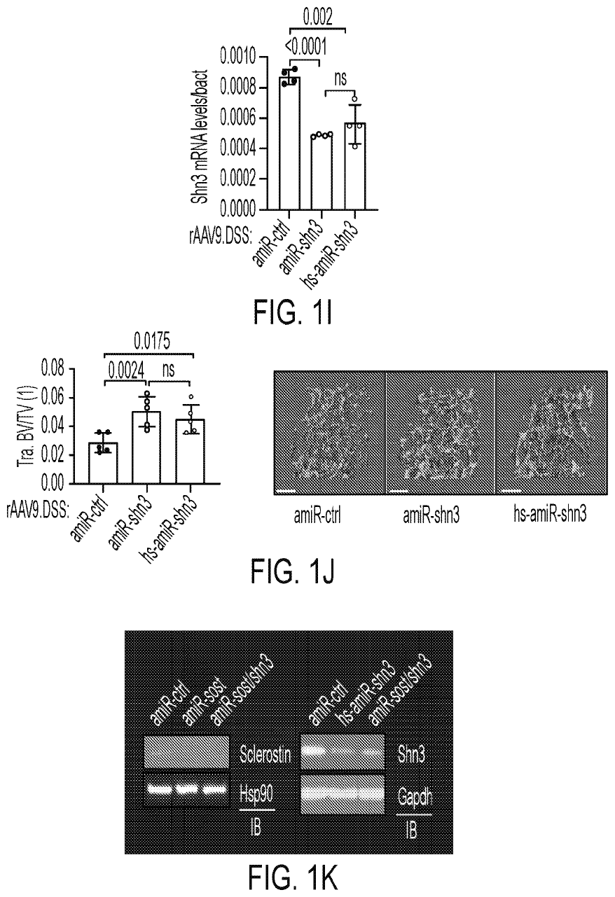

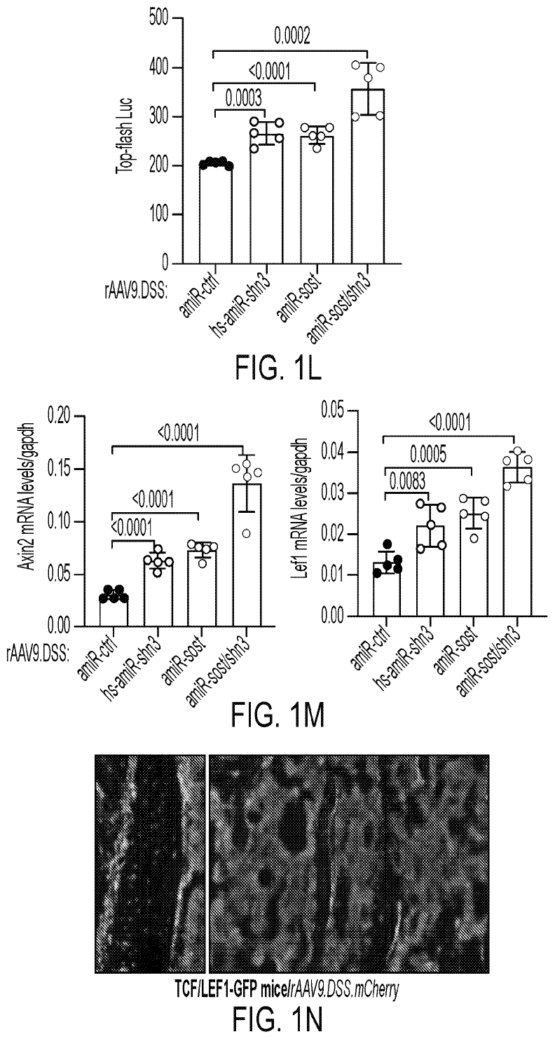

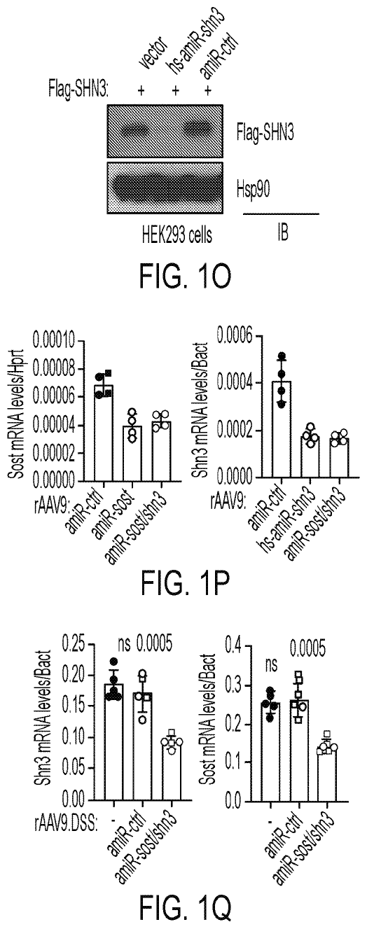

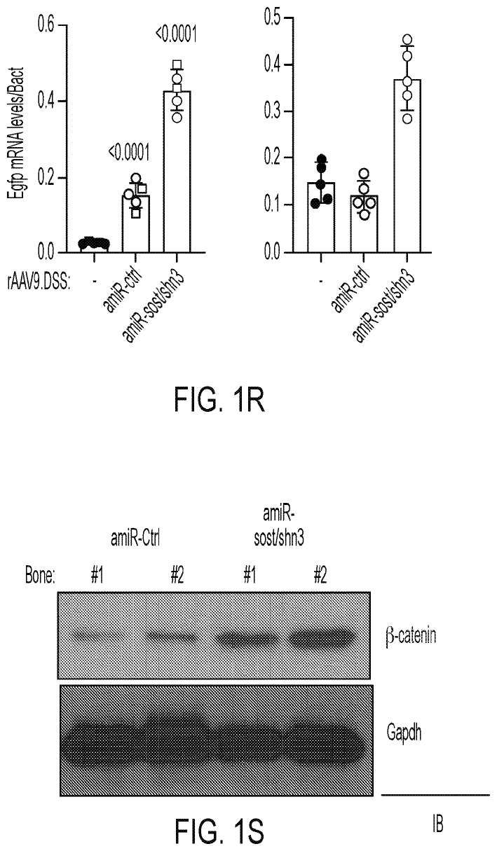

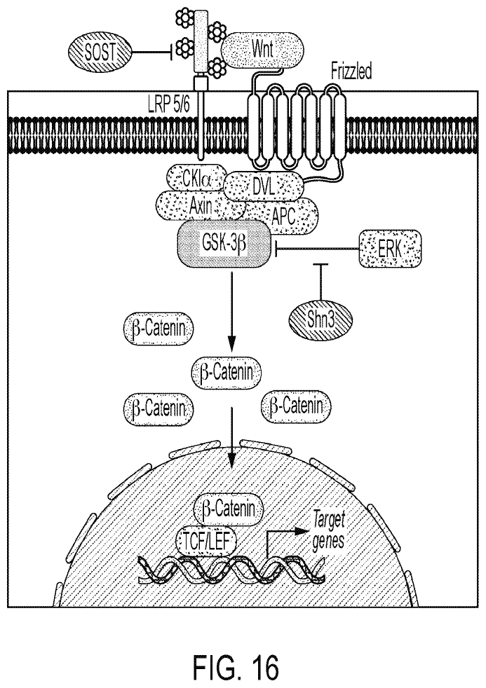

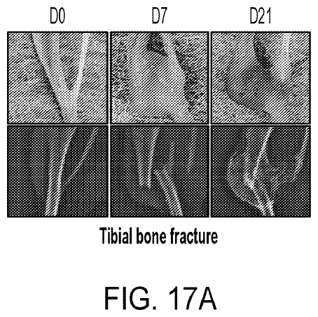

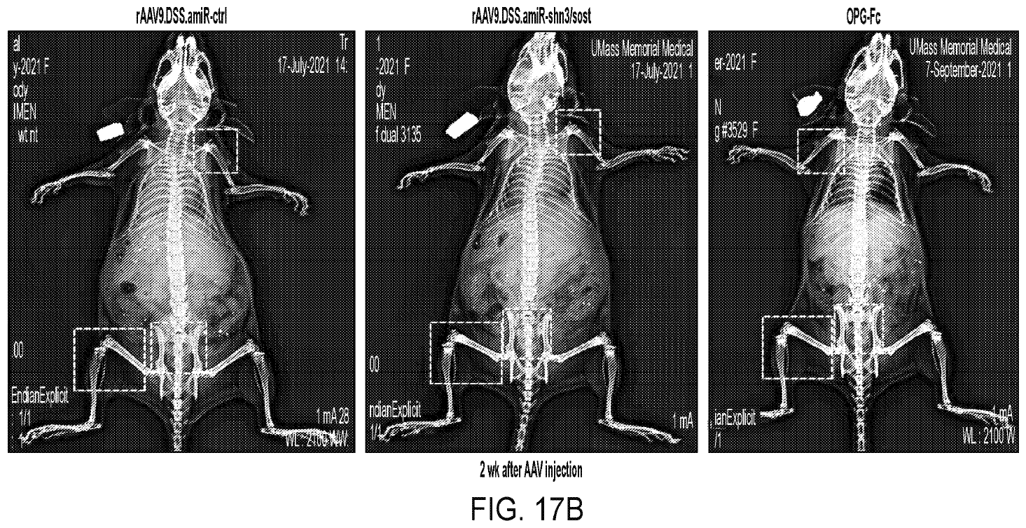

The secreted protein SOST, a WNT antagonist, has been recognized as a new target for therapeutic intervention in patients with osteoporosis. Similarly, the intracellular adaptor protein SHN3 suppresses WNT signaling in OBs. Without wishing to be bound by any theory, inhibition of SHN3 or SOST enhances WNT/β-catenin signaling, augments OB function, and promote bone formation (FIG. 16). However, this bone anabolic activity wanes over time, suggesting a negative feedback loop of the pathways of SHN3 and SOST. Accordingly, the present disclosure reveals that expression of these genes dynamically alters in response to bone remodeling or regeneration activity. As shown in FIG. 1A, aged bones in 22-month-old mice showed elevated levels of Shn3 and Sost transcripts compared to young bones in 2-month-old mice. Likewise, Shn3 and Sost expression was markedly upregulated at the late stage of bone fracture healing (day 21) while little to no change in the expression was observed at the early stage (day 7, FIGS. 1B and C, FIG. 17A). The early stage includes the inflammatory and soft callus phases and hard callus formation and bone remodeling occur at the late stage. These results imply potential involvement of WNT antagonists SHN3 and SOST in bone remodeling over the aging as well as in bone regeneration during bone fracture healing.

Intriguingly, mRNA levels of Sost were substantially increased in the long bones when treated with AAV9 carrying a shn3 silencer (AAV9.amiR-shn3) compared to AAV9 carrying control vector (AAV9.amiR-ctrl, FIG. 1D). This effect was also seen in mice lacking SHN3 in OB-lineage cells (Shn3^{Prx1}, FIG. 1E). However, Shn3 expression was not affected by AAV9-mediated silencing of Sost expression (FIG. 1F). Since SHN3 also functions as a suppressor of bone morphogenic protein (BMP) signaling that upregulates SOST expression in OBs, these results suggest a negative feedback mechanism that limits WNT signaling in OBs by enhancing BMP-induced expression of SOST.

To investigate whether co-inhibition of both factors further increases WNT signaling and bone accrual, a bone-targeting AAV9 vector that carries dual silencers targeting Shn3 and Sost was engineered (FIG. 1G). rAAV9 was previously identified by Applicant as a highly effective serotype for transduction of OB-lineage cells, including bone lining cells, endosteal OBs, and osteocytes. A bone-specific tropism of AAV9 capsid was further improved by grafting the bone-homing peptide motif (AspSerSer)₆ onto the AAV9-VP2 capsid protein (AAV9.DSS-Nter). Given that high levels of AAV-delivered short hairpin RNAs (shRNAs) induce cytotoxicity by perturbing the RNA interference machinery or exhibit significant off-target silencing, mouse miR-33-derived miRNA scaffold (artificial miRNA, amiR) was used to embed the guide strand of a small silencing RNA, which increased vector genome integrity, limited shRNA-related toxicity, and enabled efficient gene knockdown, while reducing off-target silencing by ten-fold compared to conventional shRNA constructs.

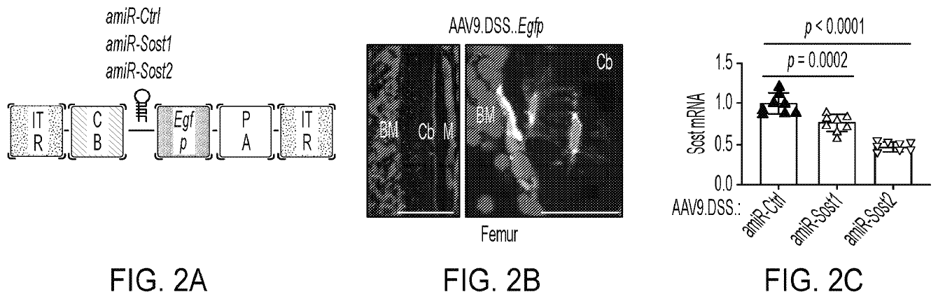

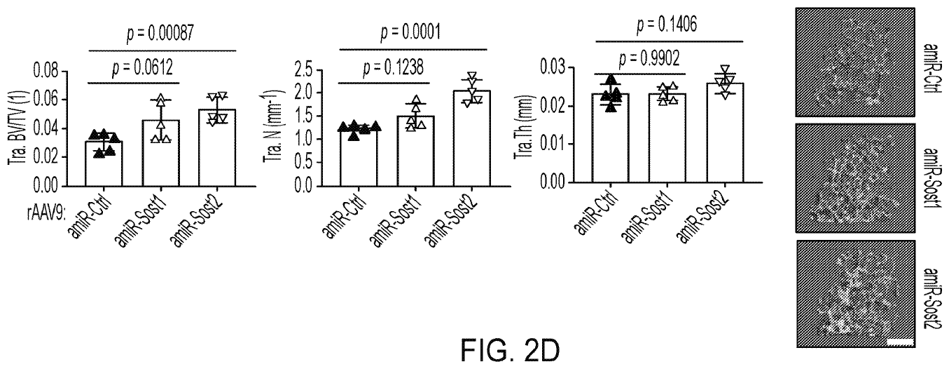

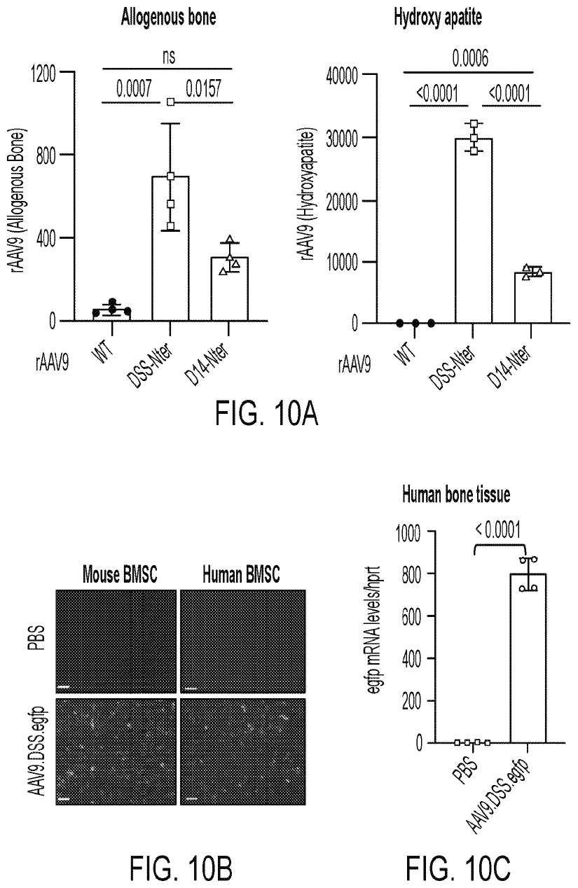

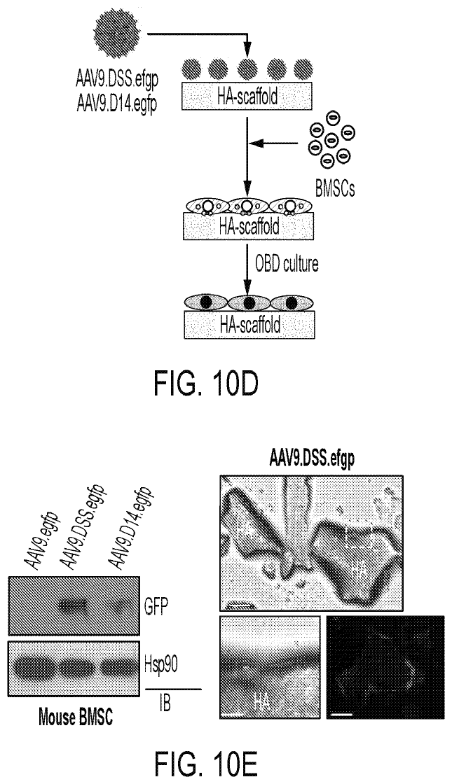

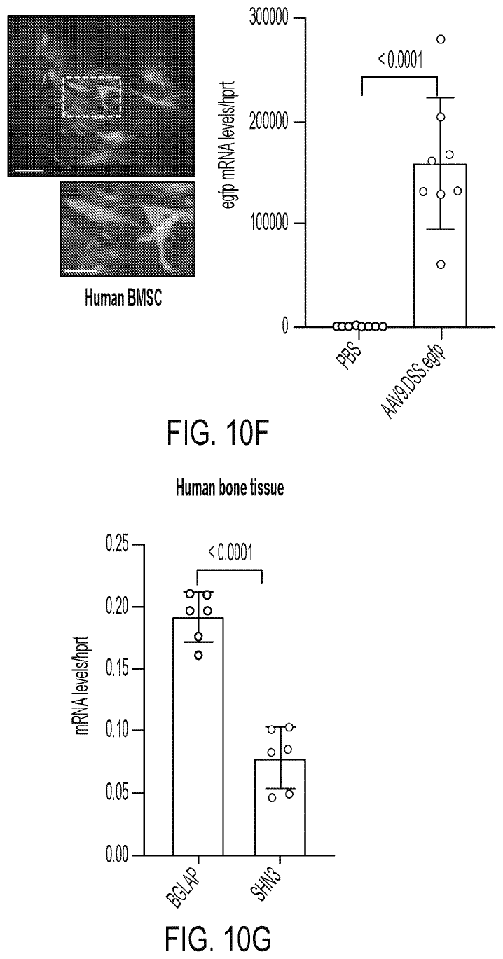

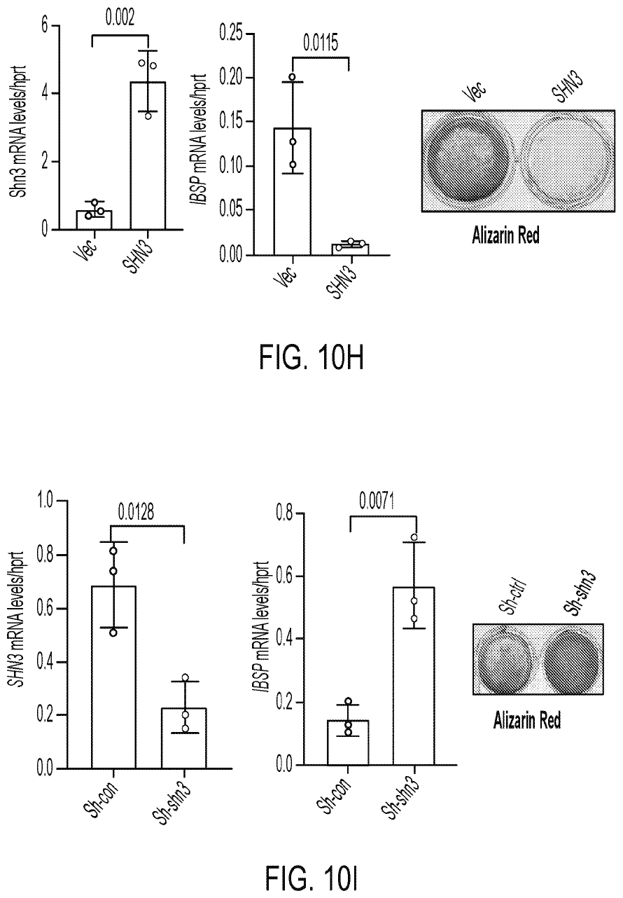

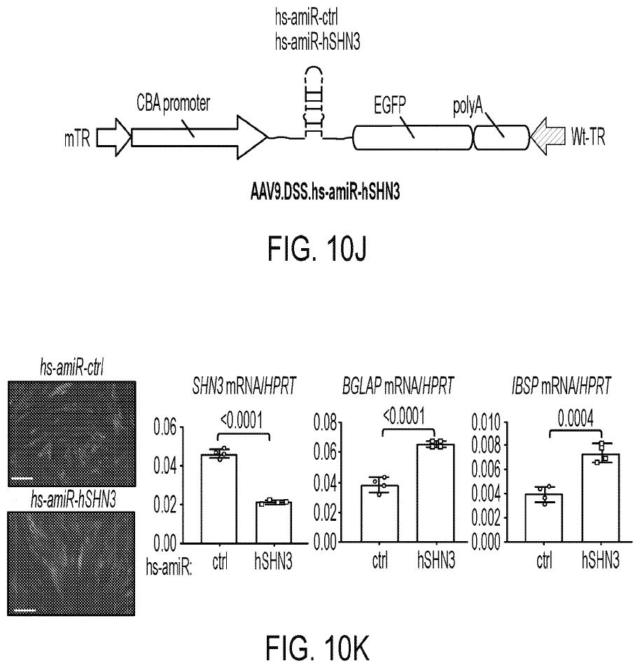

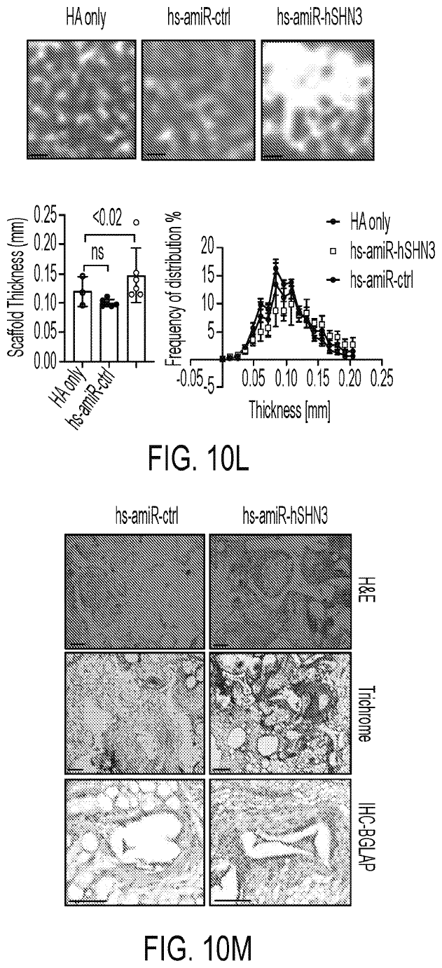

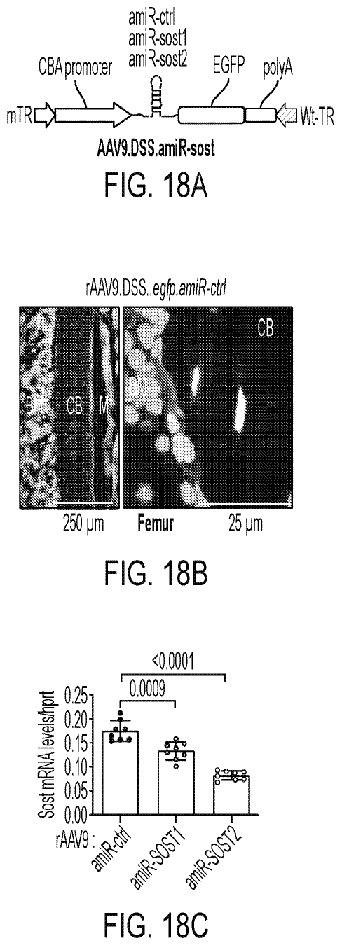

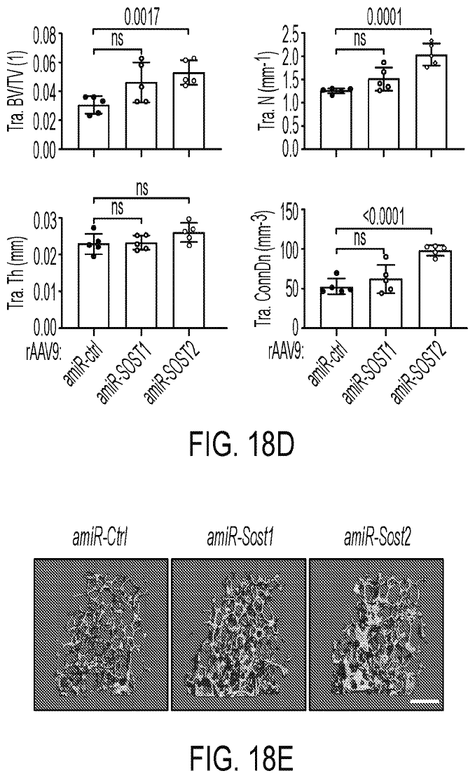

In brief, we first constructed amiR cassettes containing two mouse Sost targeting sequences (amiR-sost1, amiR-sost2) or a control (amiR-ctrl). In this design, the amiR was inserted intronically between the CB promoter and the egfp reporter gene (FIG. 2A, FIG. 18A), which allowed for visual tracking of positively transduced tissues, and the amiR cassettes were then packaged into the AAV9.DSS-Nter capsid. As expected, GFP proteins were highly expressed in osteocytes embedded in the cortical bone two months after intravenous (i.v.) injection of AAV9.DSS.egfp (FIG. 2B, FIG. 18B). When i.v. injected into 2-month-old wildtype mice, compared to amiR-ctrl-treated mice, treatment with rAAV9 carrying amiR-sost1 or amiR-sost2 resulted in ˜25 or ˜55% reduction of Sost mRNA levels in the tibia, respectively (FIG. 2C, 18C). This corresponded to an increase in trabecular bone mass of AAV-treated femurs, as shown by greater trabecular bone volume per tissue volume (Tra. BV/TV), number (Tra. N), and thickness (Tra. Th) (FIG. 2D, 18D). As shown in FIG. 1, amiR-sost-2 is referred to as amiR-sost. Without wishing to be bound by any theory, it has been reported that juxtaposition of same miRNAs or shRNAs often cause an incomplete transcription in the AAV vector genome due to formation of a tangled structure. Thus, mouse Shn3 targeting sequences were cloned into human miR-33-derived miRNA scaffold (hs-amR) that contained five different nucleotide sequences in the stem-loop site from mouse miR-33-derived miRNA scaffold (amR) (FIG. 1H). Knockdown efficiency of hs-amiR-shn3 was validated by immunoblotting analysis (FIG. 10).

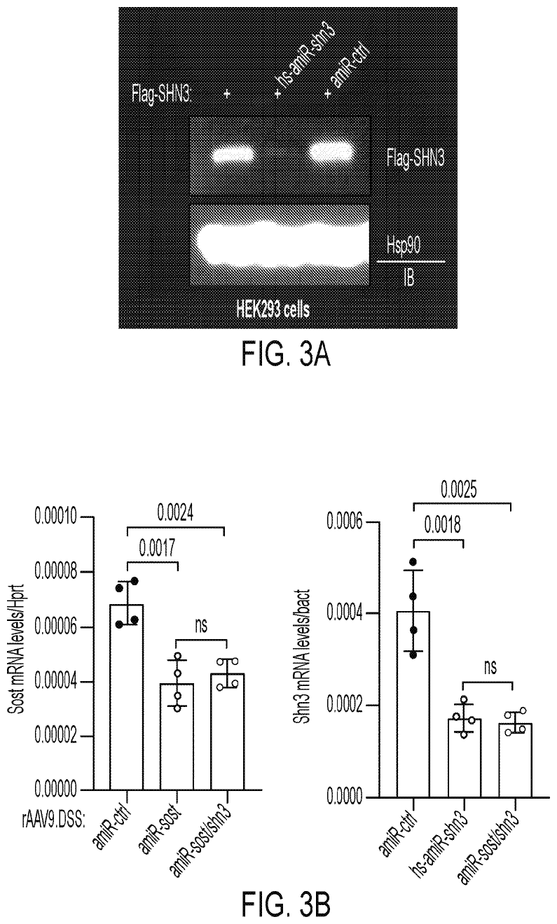

Immunoblotting analysis demonstrated that overexpression of hs-amiR-shn3 was effective for knockdown of Flag-tagged SHN3 protein in HEK293 cells (FIG. 3A). When i.v. injected into 2-month-old wildtype mice, ˜40% reduction of Shn3 mRNA levels was observed in the tibia treated with AAV9.DSS carrying amiR-shn3 or hs-amiR-shn3 (FIG. 1I), accompanied with a corresponding increase in trabecular bone mass of AAV-treated femurs (FIG. 1J). These results demonstrate that AAV9.DSS.hs-amiR-shn3 is as potent as AAV9.DSS.amiR-shn3 to silence Shn3 expression and increase bone accrual. Finally, the AAV vector genome carrying dual silencers targeting SHN3 and SOST (amiR-sost/shn3) was generated by replacing the egfp reporter with hs-amiR-shn3 in the AAV vector genome encoding amiR-sost and then, packaged into the AAV9.DSS-Nter capsid (AAV9.DSS.amiR-sost/shn3, FIG. 1G).

To test synergistic effects of AAV9.DSS.amiR-sost/shn3 on WNT/β-catenin signaling in vitro, Ocy454 osteocytic cell line was transduced with AAV vectors that carry single or dual silencers targeting Shn3 or Sost. AAV's silencing efficacy was examined in SOST-expressing Ocy454 cell line and SHN3-overexpressing C3H10T1/2 MSC-like cell line using immunoblotting (FIG. 1K) and quantitative PCR analyses (FIG. 3B, FIG. 1P), demonstrating that protein and mRNA levels of SOST or SHN3 were substantially reduced following AAV-mediated expression of amiR-sost/shn3. While single knockdown of Shn3 or Sost expression resulted in a mild increase of β-catenin-responsive luciferase activity (Top: flash Luc) in Wnt3a-treated Ocy454 cells, the luciferase activity was markedly increased when expression of Shn3 and Sost was both silenced (FIG. 1L). Similarly, expression of β-catenin target genes, Axin2 and Lef1, was slightly increased in Ocy454 cells expressing amiR-sost or amiR-shn3 and the expression was further upregulated in the presence of amiR-sost/shn3 (FIG. 1M). Thus, bone-targeting AAV vectors carrying Shn3 and/or Sost silencers are likely to function as a fine-modulator of WNT/β-catenin signaling.

### Example 3. WNT-Modulating AAV Gene Silencers Promote Synergistic Bone Formation in Mice

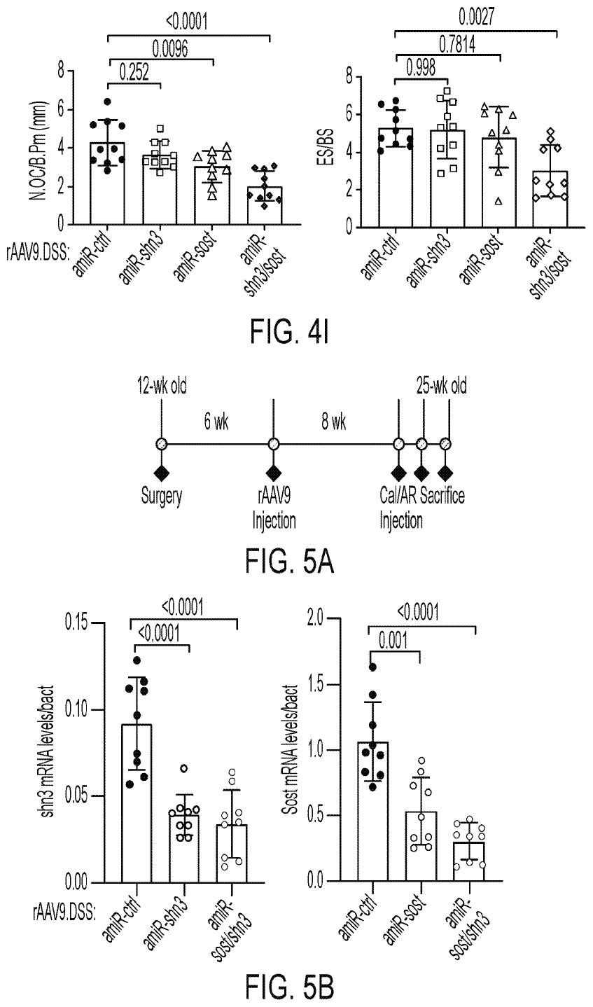

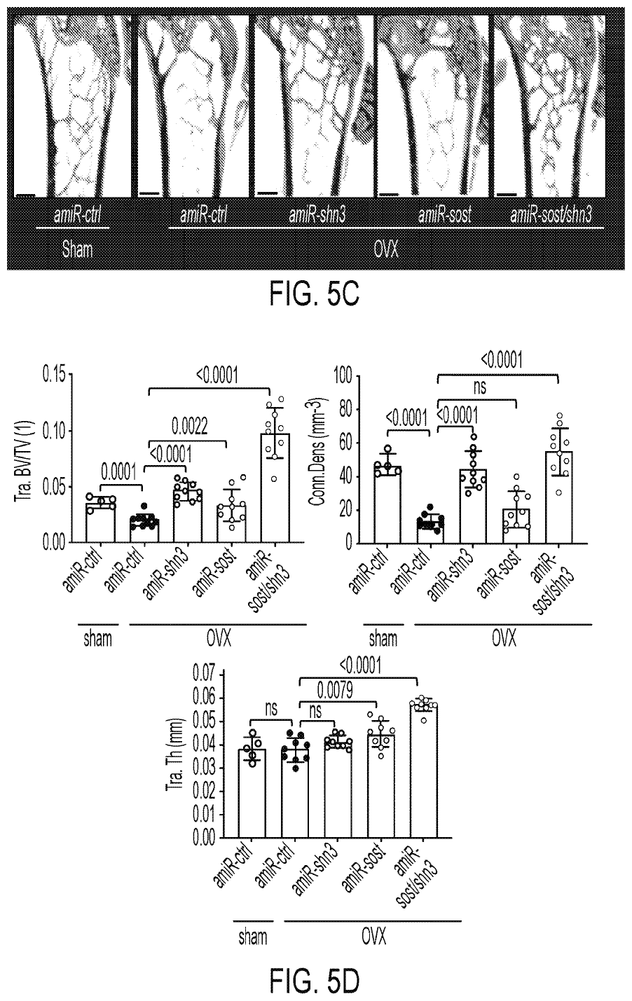

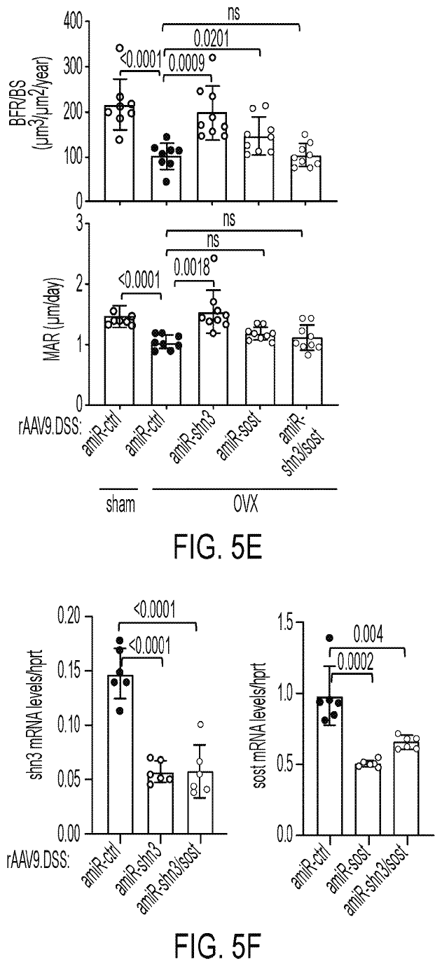

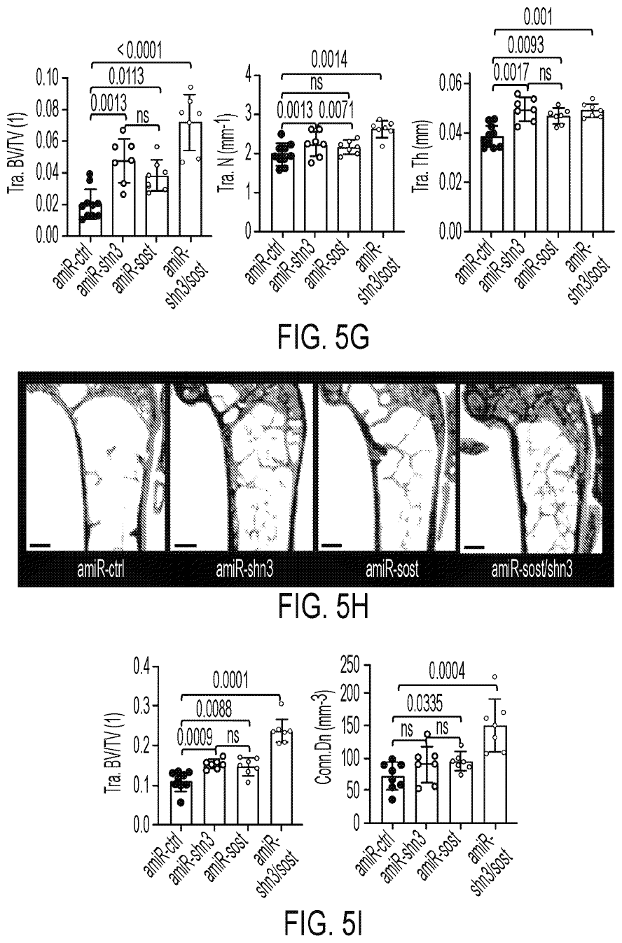

To examine the ability of WNT-modulating gene silencers to enhance bone anabolic activity in vivo, a single dose of the AAV vectors was i.v. injected into 3-month-old mice and four weeks later, knockdown efficiency and WNT signaling were assessed by quantitative PCR analysis. While amiR-sost/shn3-expressing tibia showed ˜60% knockdown of Shn3 and ˜40% knockdown of Sost expression, a similar reduction in either Shn3 or Sost mRNA levels was also observed in the tibias with a single knockdown (FIG. 5A). As observed in AAV-treated Ocy454 cells, synergistic induction of Axin2 and Lef1 in amiR-sost/shn3-expressing tibia was observed compared to mild induction by the treatment with a single silencer targeting Shn3 or Sost (FIG. 5B). This corresponded to an increase in trabecular bone mass and cortical bone thickness of AAV-treated femurs (FIGS. 5C and D). These results suggest that AAV-mediated silencing of Shn3 and/or Sost expression in the skeleton is effective to enhance WNT/β-catenin signaling and bone accrual in vivo.

### Example 4. WNT-Modulating AAV Gene Silencers Reverse Bone Loss in Osteoporosis

Postmenopausal and senile osteoporosis results in bone loss and deterioration of bone structure, increasing the risk of fractures. Bone loss in postmenopausal women is caused by enhanced osteoclast activity as a result of estrogen withdrawal. Estrogen, which is normally produced as a part of the menstrual cycle, mainly acts on osteoclasts as a negative regulator, preventing osteoclast-mediated bone resorption. On the other hand, senile osteoporosis typically develops after the age of 70 for both men and women and is a consequence of bone senescence and calcium deficiency. Ovariectomized (OVX) mice were utilized to test therapeutic effects of our WNT-modulating gene silencers in postmenopausal osteoporosis. Sham control or OVX surgery was conducted on three-month-old female mice and a single dose of AAV9.DSS carrying amiR-ctrl, amiR-shn3, amiR-sost, or amiR-sost/shn3 was i.v. injected six weeks post-surgery (FIG. 5A). Eight weeks after injection, reduced levels of Shn3 and/or Sost mRNAs were validated in AAV-treated OVX tibias (FIG. 5B). While amiR-ctrl-expressing OVX mice showed a significant reduction in trabecular bone mass relative to sham mice, this bone loss was partially and almost completely reversed by a single knockdown of Sost and Shn3 expression, respectively. When treated with AAV9.DSS.amiR-sost/shn3, trabecular bone mass in OVX femurs was further increased and even higher than that in sham femurs, shown by greater trabecular BV/TV, thickness, and connective density (FIGS. 5C and 5D). These results demonstrate that a single knockdown of Shn3 expression is more effective at reversing estrogen deficiency-induced bone loss than Sost knockdown and this effect was further improved by dual knockdown of Shn3 and Sost expression.

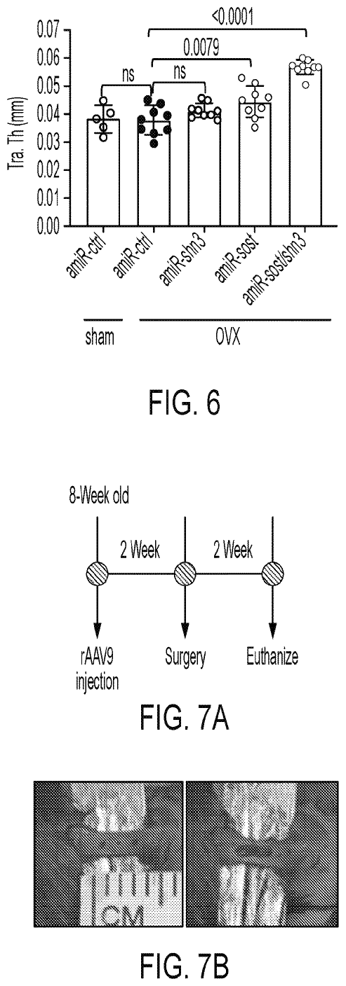

Likewise, treatment with rAAV9.DSS-amiR-shn3 in OVX mice enhanced bone formation rate (BFR) and mineral apposition rate (MAR), whereas only BFR was slightly increased in amiR-sost-expressing OVX mice (FIGS. 5E and 5F). Surprisingly, little to no increase in BFR and MAR was seen in amiR-sost/shn3 expressing OVX femurs (FIGS. 5E and 5F). Accompanied with a substantial increase in trabecular thickness (FIG. 6), these results suggest that after OB-mediated bone formation wanes over four weeks post-treatment, a decrease of OC-mediated bone resorption may be responsible for bone accrual in amiR-sost-expressing OVX mice.

Next, we tested therapeutic effects of our AAV vectors in a mouse model of senile osteoporosis. 20-month-old male mice were i.v. injected with a single dose of AAV9.DSS carrying amiR-ctrl, amiR-shn3, amiR-sost, or amiR-sost/shn3 and two months later, reduced levels of Shn3 and/or Sost mRNAs were validated in AAV-treated tibias (FIG. 5G). Unlike OVX mice, both amiR-shn3- and amiR-sost-expressing femurs showed a significant increase in trabecular bone mass, compared to amiR-ctrl-expressing femurs. Bone accrual was further increased in amiR-sost/shn3-expressing femurs, demonstrated by greater trabecular BV/TV, number, and thickness (FIGS. 3H and 3I). Notably, a similar increase of trabecular bone mass in AAV-treated lumbar vertebrae was also observed (FIG. 5J). Thus, these results demonstrate that as seen in postmenopausal osteoporosis, a dual knockdown of Shn3 and Sost expression is more effective at reversing bone loss in senile osteoporosis than a single knockdown of Shn3 or Sost expression. Collectively, WNT-modulating gene silencers using bone-targeting AAV vectors may be promising therapeutic agents for both postmenopausal and senile osteoporosis.

### Example 5. WNT-Modulating AAV Gene Silencers Promote Bone Regeneration in Uni-Cortical Bone Defect

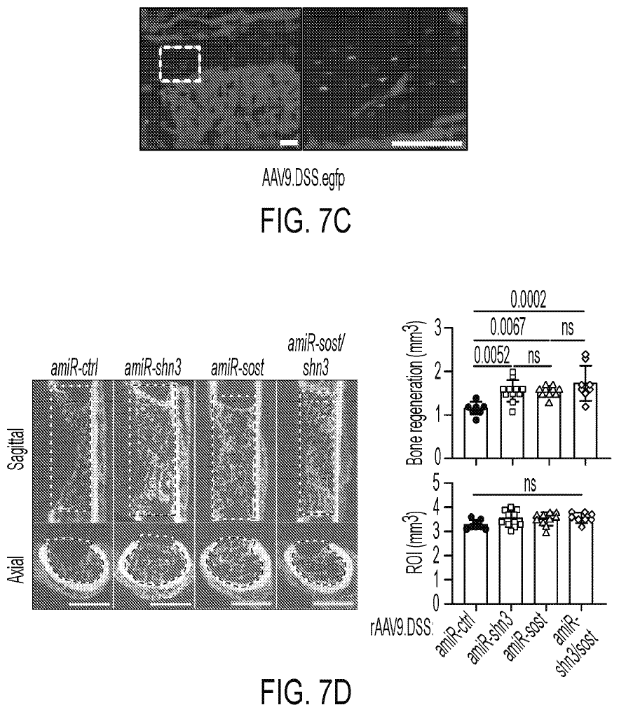

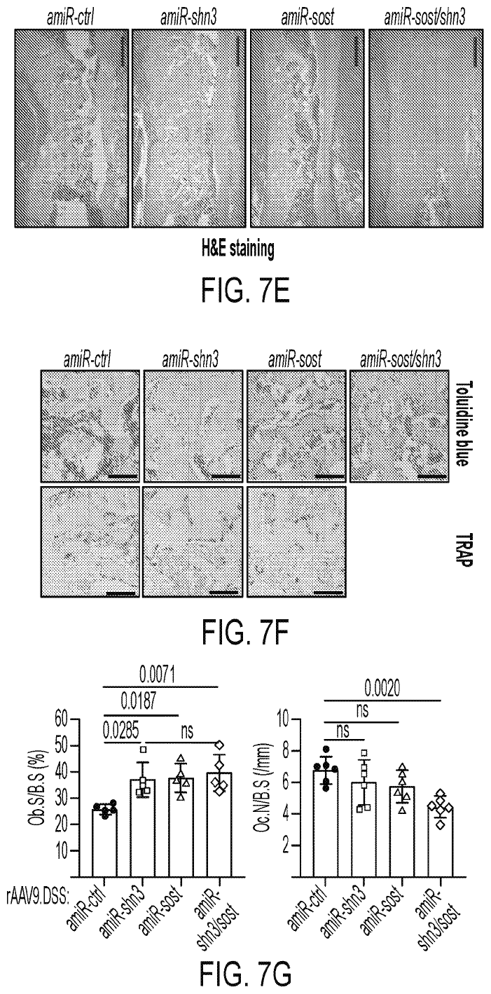

It has been shown that anabolic agents, such as PTH and anti-sclerostin antibody, can enhance bone regeneration and increase the callus volume early in the bone healing process. To investigate the ability of bone regeneration in the early healing process with WNT-modulating AAV gene silencers, the uni-cortical bone defect surgery was performed on the femur in 2-month-old mice. This surgery was designed to eliminate the mechanical instability at the bone-injury site, which is a major factor determining the callus volume. A single dose of AAV9.DSS carrying amiR-ctrl, amiR-shn3, amiR-sost, or amiR-sost/shn3 was injected two weeks before the surgery (FIG. 7A). A 3-mm length uni-cortical defect on the shaft of the femur using a 1-mm-sized motorized burr was used, preserving other cortical bones at the same level of the bone defect (FIG. 7B). The mice were euthanized and evaluated 2 weeks following the surgery. The egfp expression was detected around the bone defect by fluorescence microscopy of bone cryosection, indicating gene silencers could affect bone regeneration in the defect lesion (FIG. 7C). Reduced transcripts of Shn3 and/or Sost were validated in tibias transduced by AAV gene silencers (FIG. 7D). The volume of new bone-forming within the bone defect was measured, a significantly higher bone volume was observed in femurs treated with rAAV9.DSS-amiR-shn3, amiR-sost, or amiR-sost/shn3 than amiR-ctrl (FIG. 7E). Histological sections with hematoxylin and eosin (H&E) stain showed that the bone defect area was filled with woven bone, which exhibited a larger area in amiR-shn3, amiR-sost, or amiR-sost/shn3 expressing femur than a control (amiR-ctrl) (FIG. 7F). Histomorphometric analysis confirmed that the escalated bone formation in gene silencer treated mice was due to the increased number of osteoblast, while the decreased number of osteoclasts might also contribute in terms of rAAV9.DSS-amiR-sost/shn3 treatment (FIGS. 7G and 7H). These results demonstrate that WNT-modulation gene silencer using bone-targeting AAV vectors can promote osteoblast-mediated bone regeneration early in the bone healing process, and it is facilitated by the anti-resorptive effect in the treatment of rAAV9.DSS-amiR-sost/shn3.

### Example 6. WNT-Modulating AAV Gene Silencers Promote Bone Fracture Healing

Bone remodeling, a continuous bone replacement regulated by serial action between bone-forming osteoblast and bone-resorbing osteoclast, is necessary to achieve bone fracture union and restore biomechanical properties. It has been confirmed that WNT-modulating AAV gene silencers enhanced bone regeneration early in bone healing. However, several studies regarding the WNT-modulation with anti-sclerostin antibodies in fracture healing argued that WNT-modulation negatively affected bone remodeling and disturbed bone fracture union.

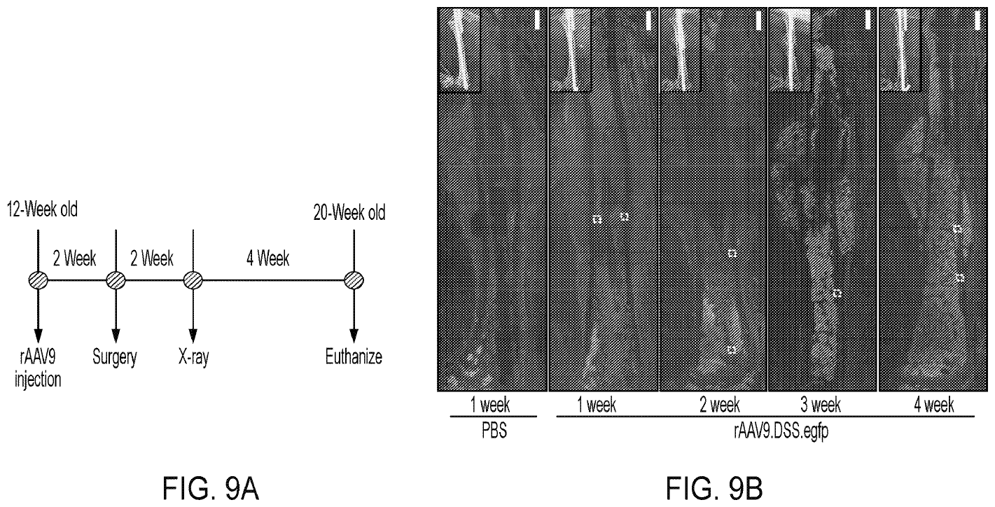

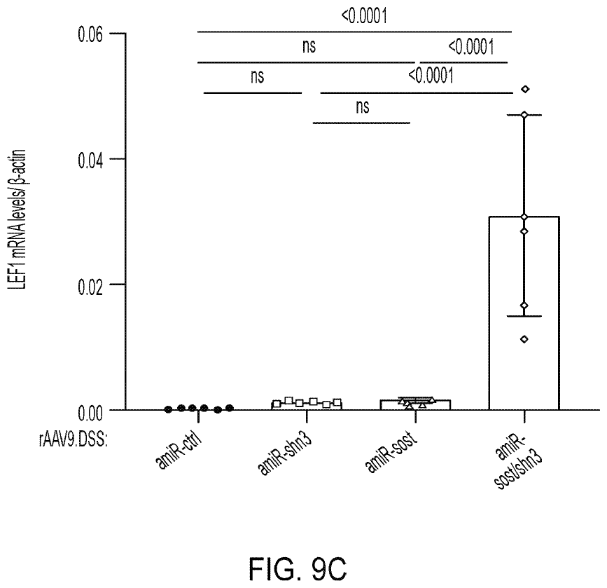

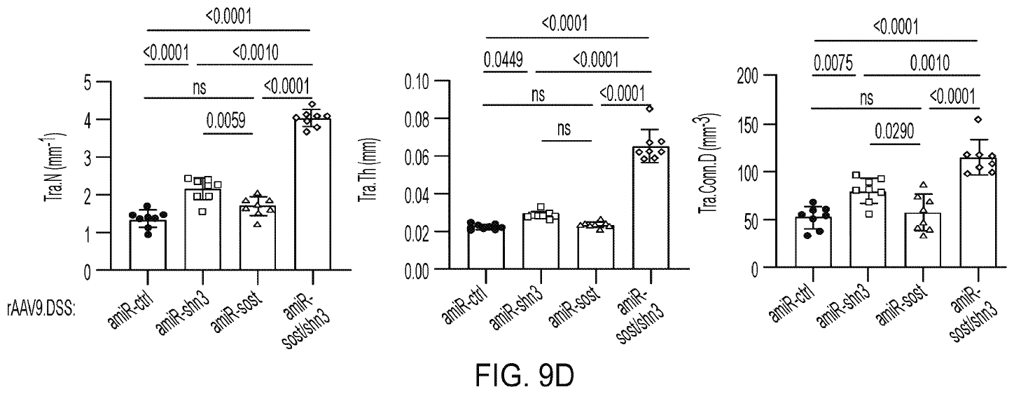

To assess the efficacy of WNT-modulating AAV gene silencers on bone fracture union, the femoral osteotomy and fixation in 3-month-old mice were conducted. Given that most of the fractures could be healed spontaneously, an intramedullary semi-rigid fixation with a 25G needle was made, which showed average 26.9% (7 of 26) union rate at 6 weeks postoperatively in the preliminary test with C57BLU6J mice. A single dose of rAAV9.DSS carrying amiR-ctrl, amiR-shn3, amiR-sost, or amiR-sost/shn3 was given intravenously (FIG. 9A). Two weeks following injection, the middle of the left femur was cut with an oscillating saw and fixed intramedullary with a 25G needle, penetrating from the patella groove of the distal femur to the greater trochanter of the femur.

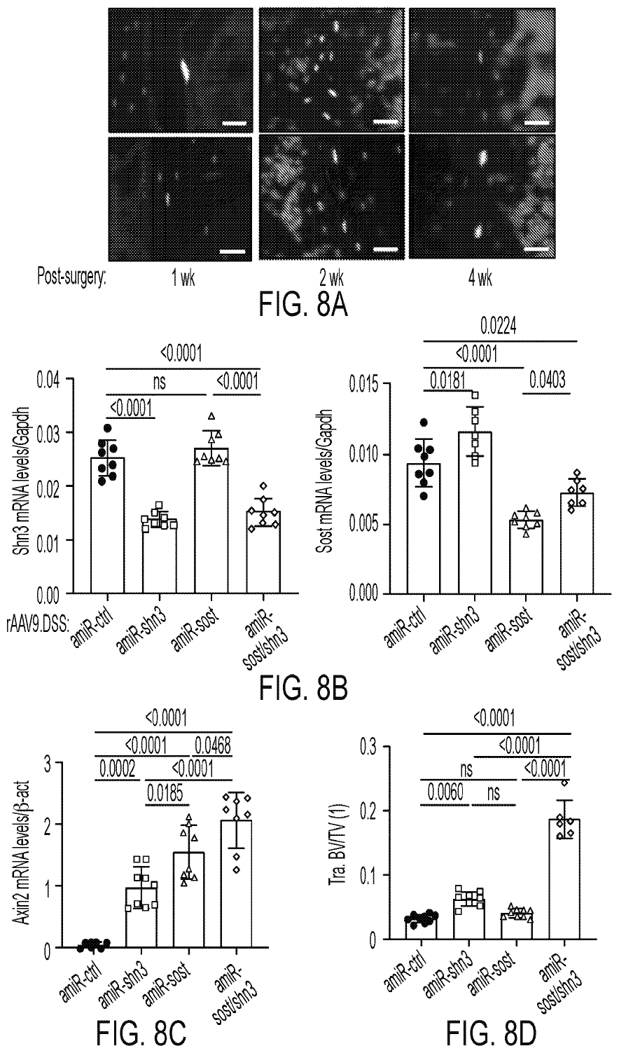

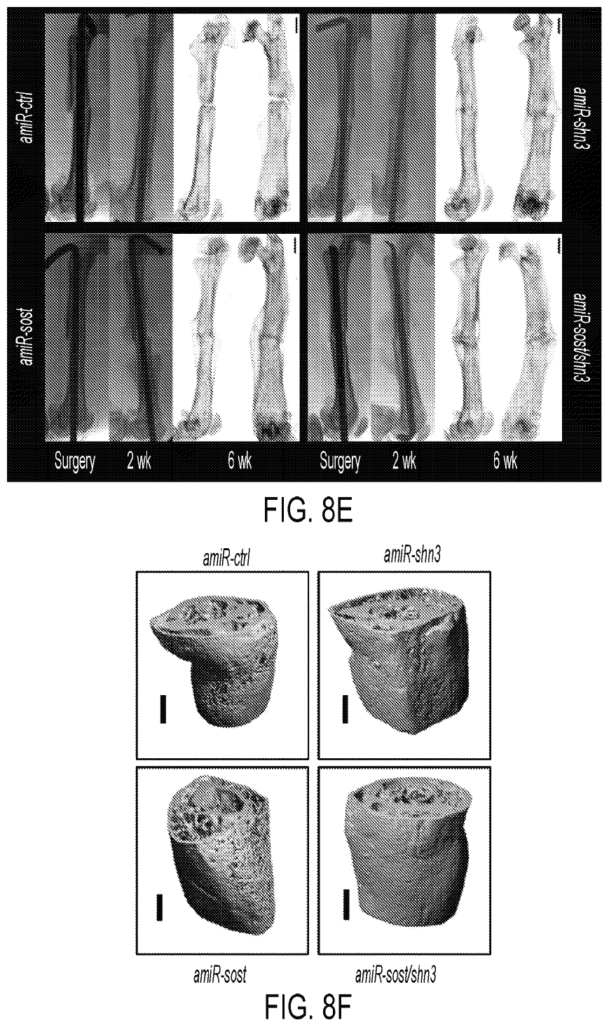

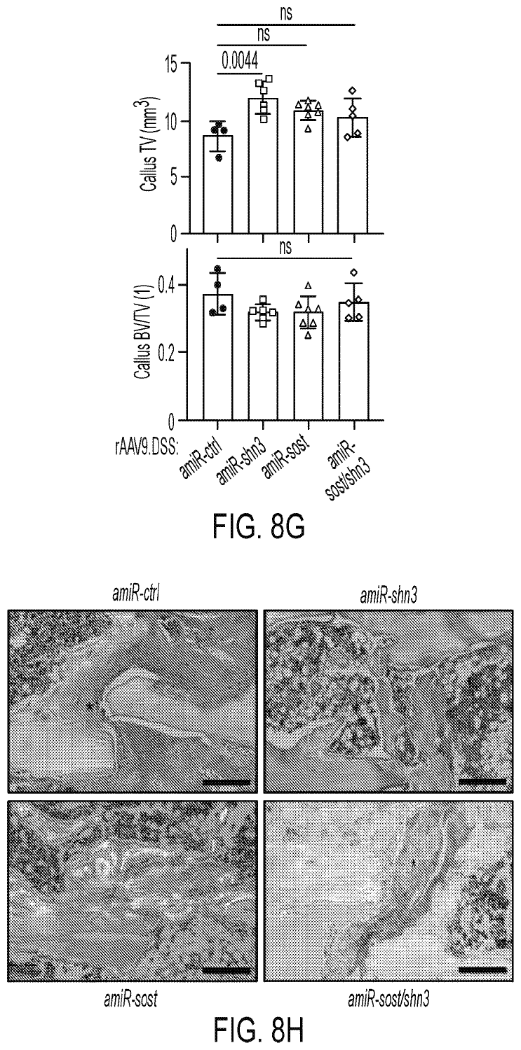

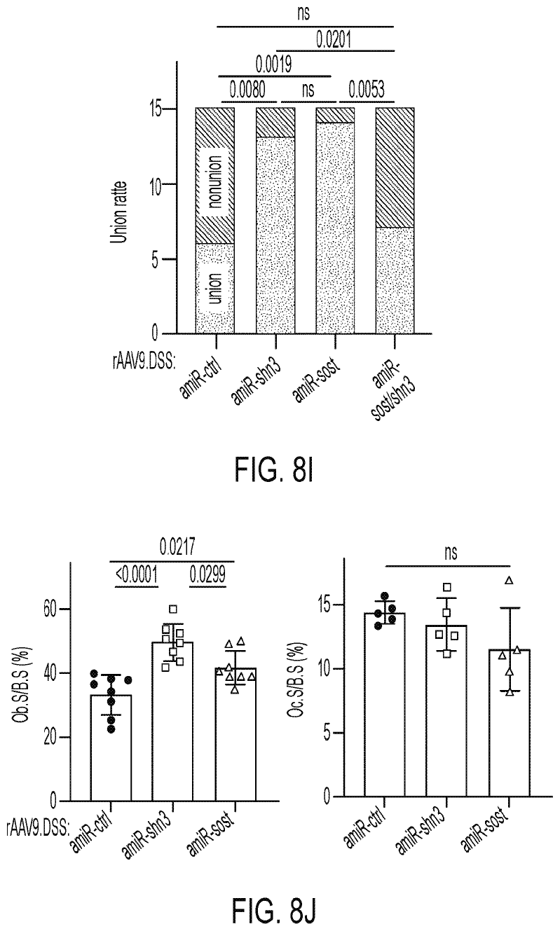

The egfp expression was monitored by fluorescence microscopy of bone cryosection near the fracture site from 1 week throughout 4 weeks postoperatively, suggesting AAV-transduced cells constitutively influenced the fracture healing process (FIG. 8A, FIG. 9B). The quantitative PCR validated suppression of shn3 and/or sost mRNA levels in AAV-treated tibias (FIG. 8B).

The expression levels of Axin2 and Lef1, β-catenin target genes, were markedly increased in treatment of rAAV9.DSS-amiR-sost/shn3, while slightly increased expression of Axin2 was seen in tibias treated with amiR-shn3 or amiR-sost, suggesting the different degree of WNT signaling pathway enhancement (FIG. 8C, FIG. 9C). Accordingly, micro-computed tomography (CT) analysis of the contralateral-side femur showed that Tra. BV/TV, Tra. N and Tra. Th were greater in amiR-sost/shn3 expressing femur (FIG. 8D, FIG. 9D). To examine the callus formation during fracture healing, a simple radiograph was performed at postfracture 2 weeks in live mice and analyzed CT at postfracture 6 weeks after euthanasia. Though early callus formation tended to increase in rAAV9.DSS-amiR-shn3, amiR-sost, and amiR-sost/shn3 in radiographs at 2 weeks, only the callus size in amiR-shn3 revealed a significant increase in CT analysis at postfracture 6 weeks (FIGS. 8E-8G).

Without wishing to be bound by any theory, WNT modulation by AAV gene silencers could affect early callus formation, but not late callus volume, which is unlikely to depend on the bone-forming activity of gene silencers but other factors such as the degree and duration of unstable condition at the fracture site. Finally, the bone fracture union rate was increased in amiR-shn3 and amiR-sost expressing femurs than amiR-ctrl, while it was surprisingly not improved in amiR-sost/shn3 expressing femur (FIG. 8H). Histologic section with H&E stain exhibited the connection between ends of the fracture by woven bone in rAAV9.DSS-amiR-shn3 and amiR-sost, suggesting active bone remodeling (FIG. 8I). Histomorphometric analysis showed an increased osteoblast surface per bone surface (Ob.S/B.S) in all three gene silencers than control. However, osteoclast surface per bone surface (Oc.S/B.S) was decreased only in rAAV9.DSS-amiR-sost/shn3 (FIG. 8J). Collectively, it is inferred that reduced osteoclast activity would negatively affect bone remodeling and lead to worse bone fracture union in rAAV9.DSS-amiR-sost/shn3. rAAV9.DSS-amiR-shn3 or amiR-sost activates osteoblast-mediated bone formation without disturbing osteoclast-mediated bone-resorbing, resulting in apparent improvement of bone fracture union.

### Example 7. Development of a Bone Anabolic Human Skeletal Organoid for Critical-Sized Bone Defect

The healing of critical-sized skeletal defects (CSD) remains one of the most challenging problems in orthopedic management because CSDs are unable to heal without interventions such as insertion of a bone graft. Although autogenous bone graft with excellent osteoconductive, osteoinductive, and osteogenic activities is a gold standard treatment, harvest of autologous bone is limited by donor site morbidity and amount. Thus, allogenous bone graft has been developed to avoid these issues inherent with autograft harvest. However, they also display limited intrinsic osteogenic potential, with allograft fractures commonly resulting in persistent nonunion.

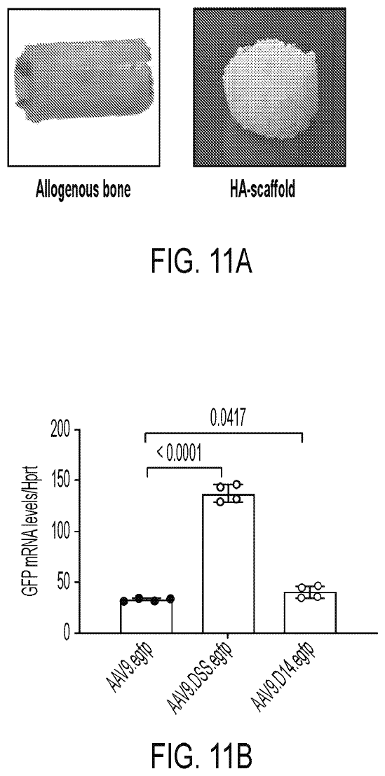

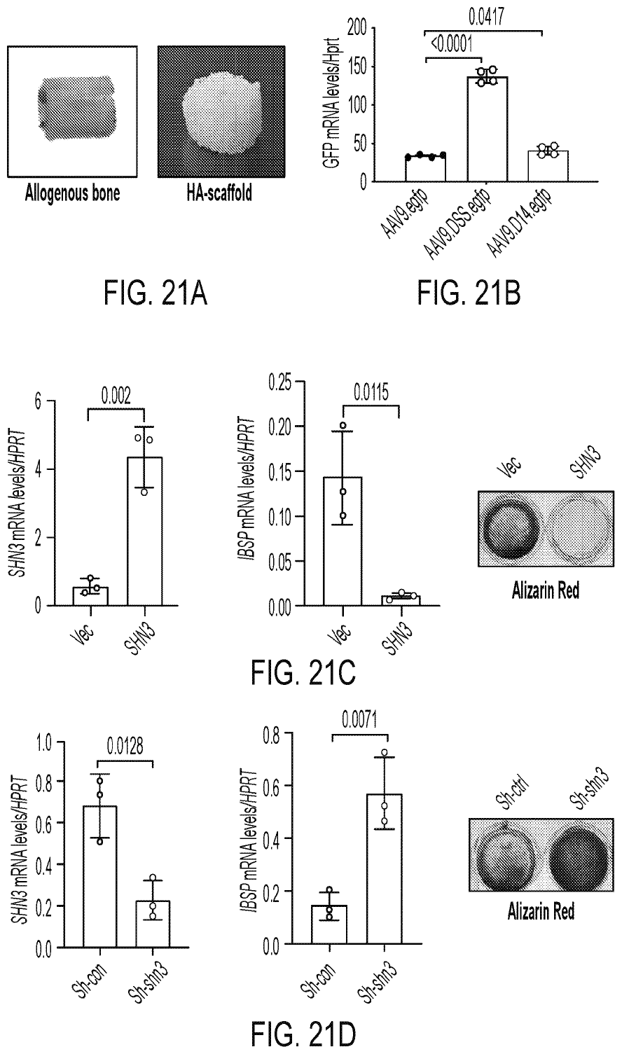

To improve allograft outcomes, development of new adjunct methods to enhance bone formation along with promoting integration with the surrounding skeletal tissue is needed. One potential approach is to implant an OB-seeded allograft (skeletal organoid) into CSD injury sites, in which our WNT-modulating gene silencers may promote bone regeneration of the allograft due to augmented OB function. To attach our AAV vectors to allogenous bone materials, bone-targeting AAV9 capsids were utilized by grafting the peptide motifs homing to OC-enriched bone-forming surfaces, (Asp-Ser-Ser)₆ or OC-enriched bone-resorbing surfaces, (Asp)₁₄, onto the N-terminus of the VP2 subunit of the AAV9 capsid protein. rAAV9, rAAV9.DSS-Nter (rAAV9.DSS), or rAAV9.D14-Nter (rAAV9.D14) vectors were incubated with decellularized allogenous bone or hydroxyapatite (HA)-based scaffold (FIGS. 11A and 21A) and their binding affinity was assessed by measuring AAV's genome copies (GCs). While little to no GCs of rAAV9 in the decellularized bone and HA-scaffold were detected, GCs of rAAV9.DSS were markedly increased relative to those of rAAV9.D14 (FIG. 10A, FIG. 11B, FIG. 21B), demonstrating that the AAV9.DSS-Nter capsid can enhance in vitro binding affinity to allogenous bones via a major organic component HA. Capsid grafting of this peptide motif did not affect rAAV9's transduction efficiency in mouse and human bone marrow-derived stromal cells in vitro (BMSCs, FIG. 10B) and human bone tissue ex vivo (FIG. 10C). Next, the HA-scaffolds attached with rAAV9.DSS or rAAV9.D14 were incubated with mouse BMSCs and their transduction efficiency was assessed by EGFP expression (FIG. 10D), demonstrating that rAAV9.DSS is more effective for BMSC transduction than rAAV9.D14 (FIG. 10E). Likewise, the rAAV9.DSS-attached HA-scaffold was able to transduce human BMSCs as well (FIG. 10F), suggesting that the rAAV9.DSS-attached HA-scaffold may provide an optimal environment for human BMSC culture to develop a human skeletal organoid.

SHN3 is a large (>2,000 amino acids) intracellular adaptor protein, highly expressed in a human bone tissue (FIG. 10G) and BMSCs (FIG. 10I), functions as an inhibitor of WNT signaling in OBs. As SHN3-deficient mice display a progressive increase in bone mass due to augmented OB activity, overexpression of SHN3 similarly ablated OB differentiation in human BMSCs (FIG. 10H), whereas OB differentiation was markedly increased by shRNA-mediated knockdown of SHN3 expression (FIG. 10I). Human miR-33-derived miRNA scaffold (hs-amR)-based cassettes were engineered to silence the expression of human SHN3 mRNA (FIG. 10J).

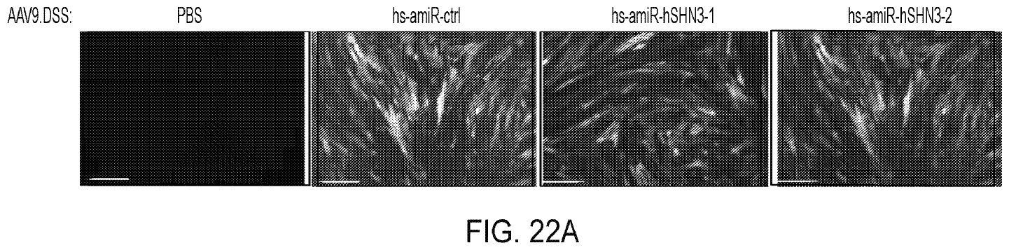

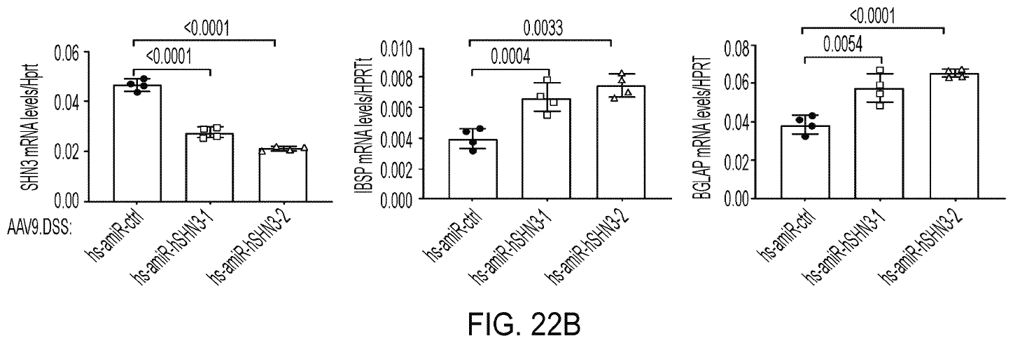

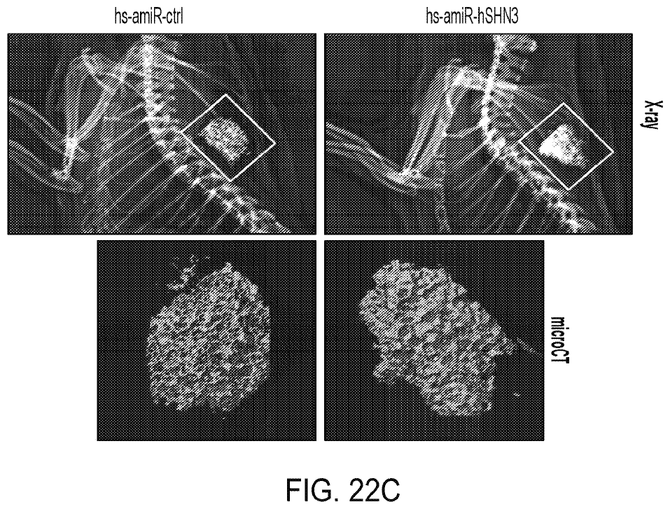

As SHN3 functions as an inhibitor of WNT signaling in osteoblasts, overexpression of SHN3 similarly ablated osteoblast differentiation in human BMSCs (FIG. 21C), whereas osteoblast differentiation was markedly increased by shRNA-mediated knockdown of SHN3 expression (FIG. 21D). As disclosed herein, using human miR-33-derived miRNA scaffold-based cassettes to silence the expression of human SHN3 mRNA (hs-amR-hSHN3), a bone-specific AAV vector carrying a humanized SHN3 silencer was developed. Two rAAV9.DSS.hs-amR-hSHN3s targeting different sequence of human SHN3 mRNA (hs-amR-hSHN3-1 and ˜2) were generated and transduction and knockdown efficiency and osteogenic potentials in human BMSCs were validated (FIGS. 22A and 22B, FIG. 10H). Hereafter and in FIG. 10, hs-amiR-hSHN3-2 is referred to amiR-hSHN3. To test bone anabolic activity of rAAV9.DSS.hs-amiR-hSHN3 in vivo, human BMSCs were seeded onto the HA-scaffolds attached with rAAV9.DSS carrying hs-amiR-ctrl or hs-amiR-hSHN3 and then, these scaffolds were implanted into interscapular fat pads of immune-compromised SCID mice. Four weeks later, skeletal analyses were performed by microCT and histology (FIG. 22C). Compared to hs-amiR-ctrl, hs-amiR-hSHN3-expressing HA-scaffold showed enhanced bone formation, as shown by a significant increase in scaffold thickness, collagen production, and numbers of osteocalcin-positive osteoblasts (FIGS. 101 and 10J, FIG. 22C). These results suggest that the humanized SHN3 silencer-expressing HA-scaffold is effective to promote bone formation and may be useful for the treatment of critical sized bone defect as a human skeletal organoid with a potent bone anabolic activity.

### Example 8. Bone-Attached WNT-Modulating AAV Gene Silencers Promote Healing of the Critical-Sized Skeletal Defect

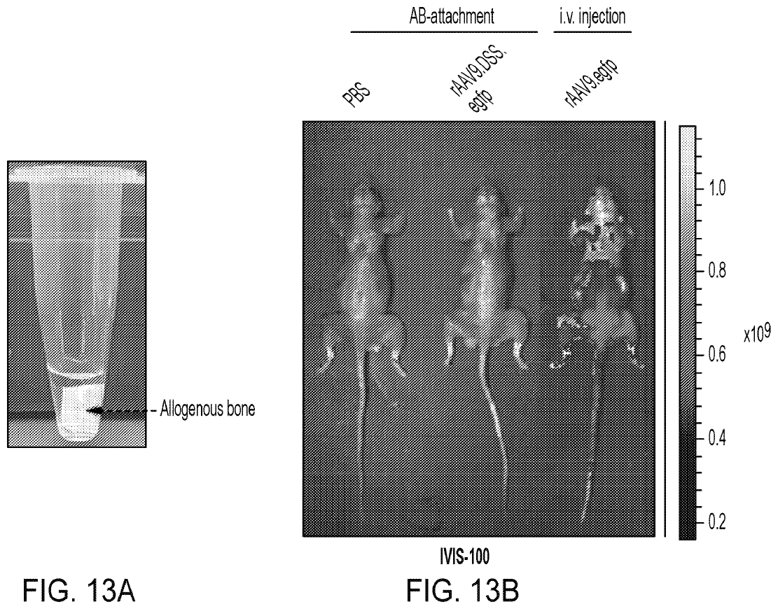

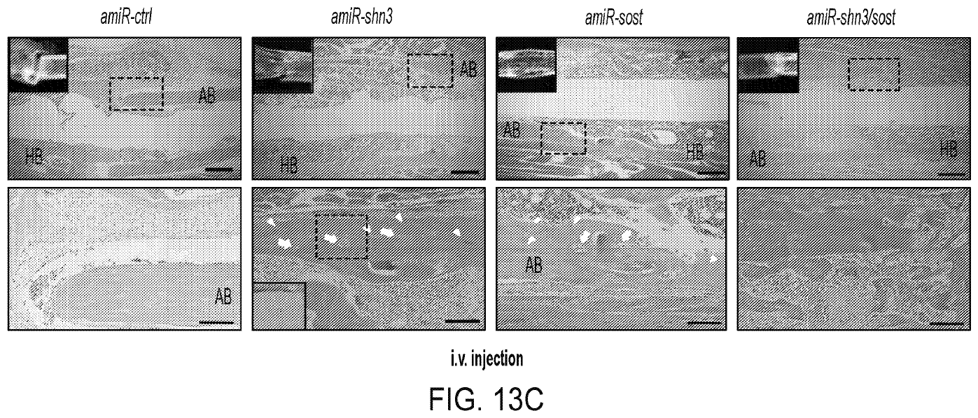

It had been found by Applicant that WNT-modulation with rAAV9.DSS carrying amiR-shn3 or amiR-sost can enhance bone healing and improve fracture union. Given that enhanced bone healing capacity is mainly required to manage further challenged skeletal injury, the critical-sized skeletal defect with C57BL/6J mice was designed. The localized administration of AAV gene silencers as set up in accordance with previous studies. It has been reported by a few other studies that local gene delivery in critical-sized skeletal defect by ex vivo attachment of AAV vectors to the allogenous bone (AB) was attempted. The AB was also used to fill the bone defect site, AB-attached WNT-modulating AAV gene silencers were produced by incubating the AB in 30 μl of sterile PBS containing DSS.rAAV9 (1.0+13 GC/ml) for 30 min at room temperature (FIGS. 11A, 13A). It was investigated whether these bone-attached gene silencers could enhance bone healing in the critical-sized skeletal defect while reducing distant off-target distribution.

Before attaching AAV to the AB, the AB-binding affinity in vitro with rAAV9.DSS, rAAV9.D14 ((Asp)₁₄), and rAAV9 was tested. The bone-targeting peptide motif enhanced the AAV's AB-binding affinity. Genome copies (GC) of AAV9.DSS was markedly increased relative to those of rAAV9.D14, which showed a higher GC than rAAV9. Likewise, in vitro test of hydroxyapatite (HA)-binding affinity showed significantly increased GC levels in rAAV9.DSS and rAAV9.D14 than rAAV9 (FIG. 10A). These results demonstrated that rAAV9.DSS has a better affinity to AB, and AAVs attach to the HA composition within the AB. We also examined bone-attached AAV's transduction test in vitro, and egfp expression was detected by fluorescent microscopy directly on the HA-attached AAV after incubating with mouse BMSCs and fluorescent microscopy after cryosection. The increased egfp expressions in AAV9.DSS and AAV9.D14 were also confirmed by immunoblotting and quantitative PCR (FIG. 10D, FIG. 11B).

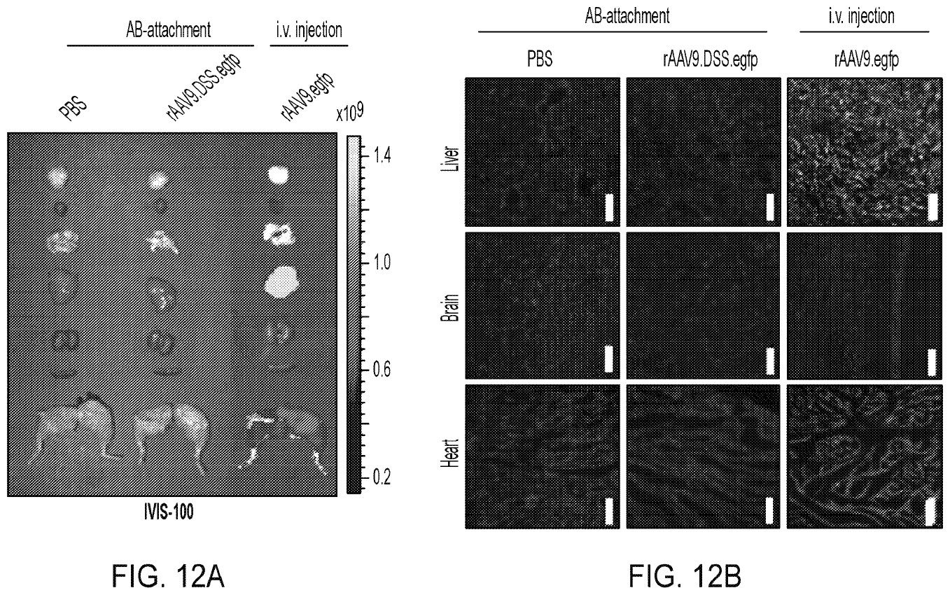

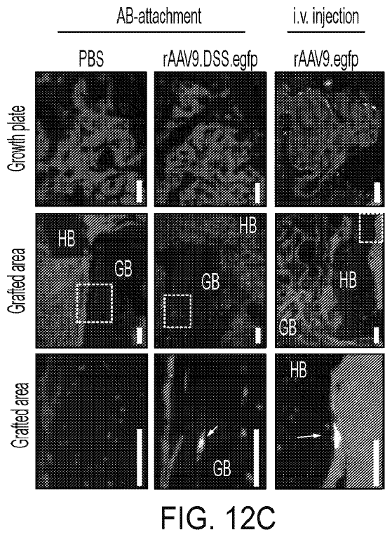

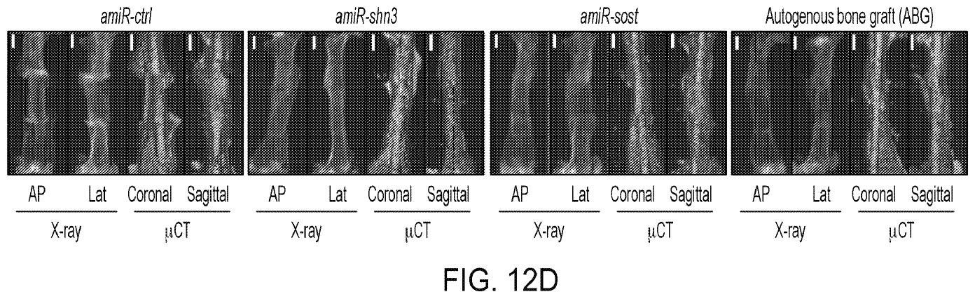

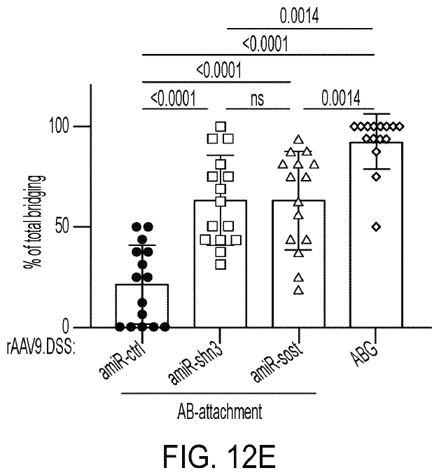

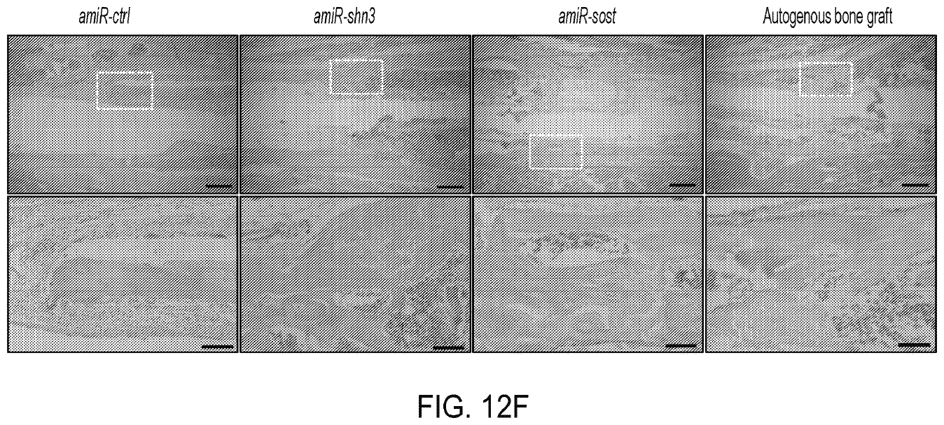

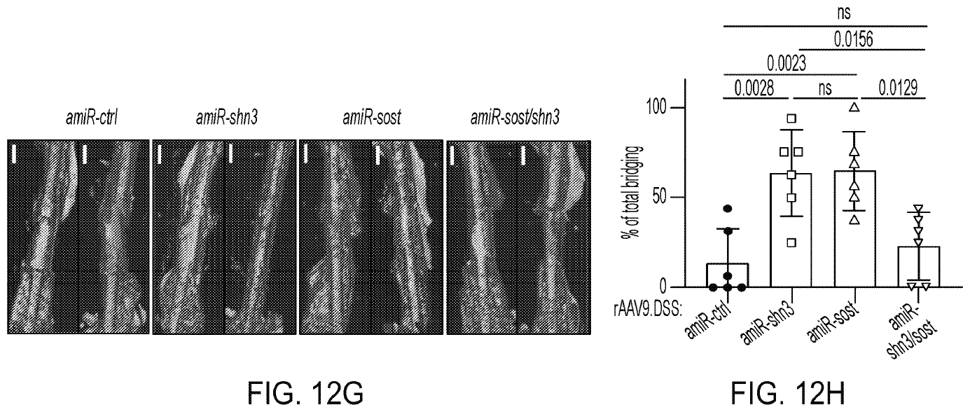

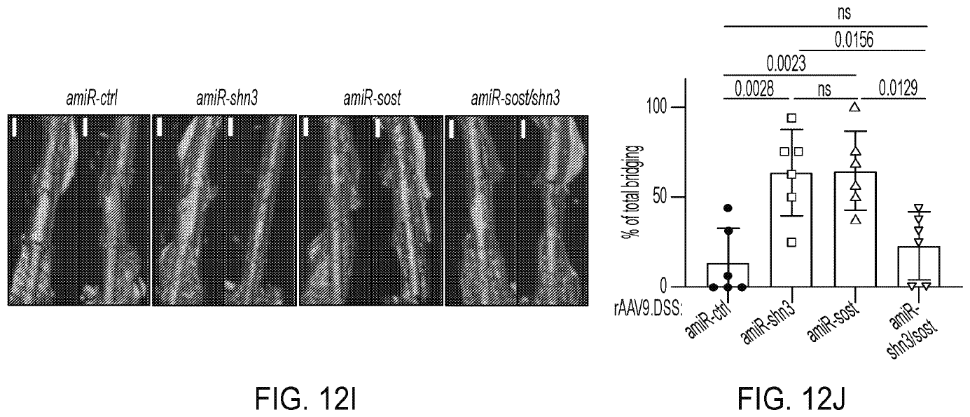

To investigate the off-target distribution of AB-attached AAV in vivo, the AB attached with rAAV9.DSS-egfp was grafted into the critical-sized femoral bone defect of 3-month-old mice. The AB incubated in PBS was used for the negative control, while rAAV9-egfp was intravenously injected 2 weeks before the AB (no treated) graft for the positive control. Two weeks following the surgery, no egfp expression in distant organs was monitored by IVIS-100 optical imaging and by fluorescence microscopy of bone cryosections in AB-attached rAAV9.DSS-egfp treated mice, while systemic injection of rAAV9-egfp showed higher egfp expression in the liver and heart (FIG. 12A, FIG. 13B). On the other hand, egfp expression was detected in the grafted bone treated with bone-attached rAAV9.DSS by fluorescence microscopy of bone cryosections, suggesting that bone-attached AAVs can transduce recruited cells and regulate target-gene expression during bone defect healing (FIG. 12C).

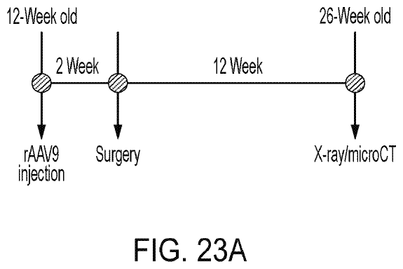

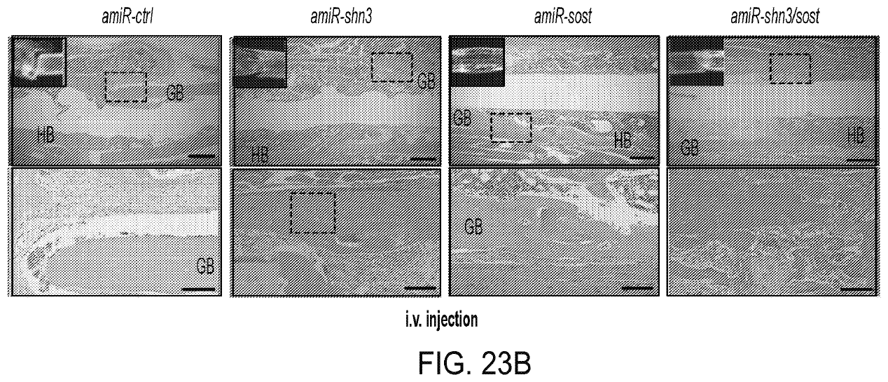

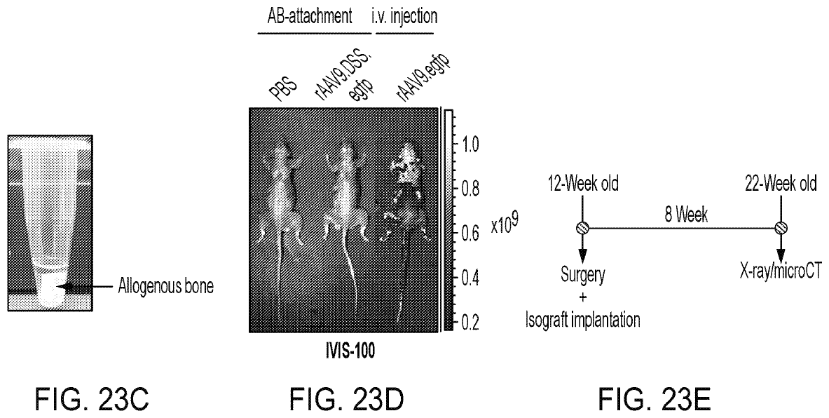

Next, to evaluate the bone healing efficacy of bone-attached WNT-modulation AAV gene silencers in the critical-sized skeletal defect, the AB graft with bone-attached rAAV9.DSS-amiR-ctrl, amiR-shn3, or amiR-sost in 3-month-old mice was carried out and compared to the total bone bridging ratio. Autogenous bone graft was also compared as a positive control. The percentages of total bridging were increased in amiR-shn3, and amiR-sost treated femurs than amiR-ctrl, though they were lower than those of autogenous bone graft (FIG. 12D, 12F). The percentage of total bridging between the implanted isograft and host femur was measured by microCT eight weeks post-surgery (FIG. 23A), demonstrating that single silencing of Shn3 or Sost improved the unionization by ˜60%, whereas little to no improvement was made by dual silencing (FIGS. 121 and 12J, 23B). Histologic sections with H&E stain showed woven bone connecting between the host bone (HB) and graft bone (GB) in bone-attached rAAV9.DSS-amiR-shn3, amiR-sost, and autogenous bone graft (FIG. 12E). The percentages of total bridging in bone-attached gene silencers (near 60%) were similar to those of systemic injection of gene silencers (FIG. 12G, 12H). These results suggest that as seen in bone fracture healing, a single silencer targeting Shn3 or Sost is more effective for healing of critical-sized femoral defect than a dual silencer.

The single silencers were also examined to evaluate its capability to promote healing of critical-sized femoral defect when directly delivered into the osteotomy site. To test transduction efficiency of rAAV9.DSS-attached isograft to host osteoblasts in the osteotomy site, the decellularized isografts were incubated with PBS or rAAV9.DSS.egfp (FIG. 23C) and then, implanted into the osteotomy site of 3-month-old wildtype mice. Three weeks later, tissue distribution of rAAV9.DSS was monitored by IVIS-100 optical imaging and fluorescence microscopy (FIG. 23D).

Conclusively, these outcomes document that bone-attached WNT-modulation AAV gene silencers can improve bone healing in the critical-sized skeletal defect while reducing the off-target distribution to distant organs.

### Example 9. WNT-Modulating Gene Silencers Increase Bone Accrual In Vivo

To test transduction efficiency of the bone-specific AAV vector to WNT-receiving osteoblasts and osteocytes, mCherry-expressing AAV9.DSS vector was i.v. injected into the transgenic TCF/Lef1:H2B-GFP reporter mice, which express a fused protein of histone 2B and GFP in responsive to WNT stimulation (ref). mCherry expression was detected in GFP-positive bone lining osteoblasts and osteocytes embedded in the bone matrix 2 weeks post-injection (FIG. 1N), demonstrating that systemic delivery of AAV9.DSS vector was effective for the transduction of WNT-receiving cells in the bone. Next, a single dose of AAV9.DSS.amiR-sost/shn3 was i.v. injected into these reporter mice to examine its ability to enhance WNT/β-catenin signaling in vivo. Accompanied with ˜45% knockdown of Shn3 and Sost expression (FIG. 1Q), dual silencing of Shn3 and Sost markedly increased WNT/β-catenin signaling in the tibia, as shown by upregulated expression of GFP and Lef1 (FIG. 1R) and β-catenin (FIG. 1S). These results demonstrate that AAV-mediated silencing of Shn3 and Sost is effective to enhance WNT/β-catenin signaling in the bone.

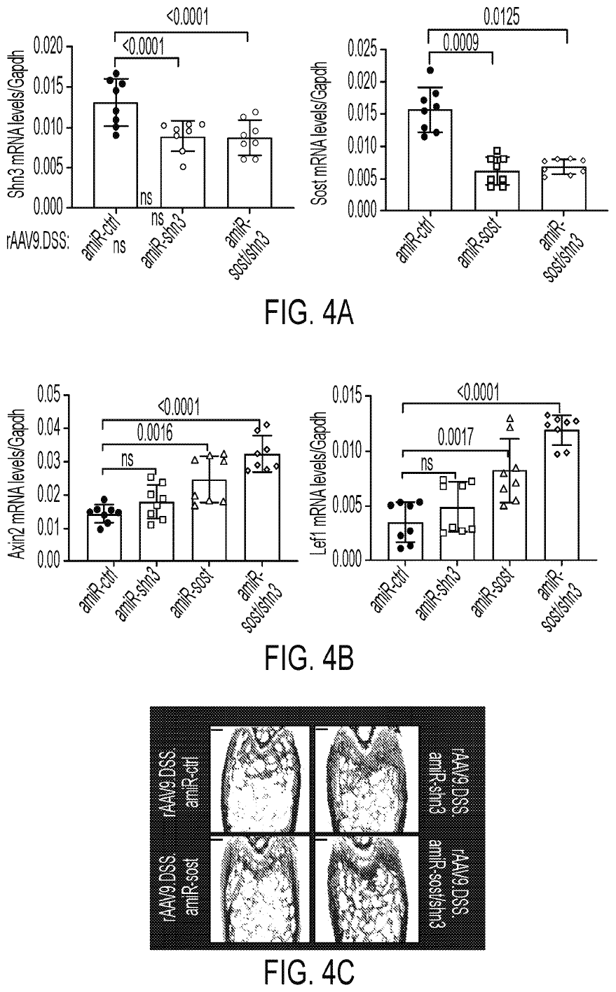

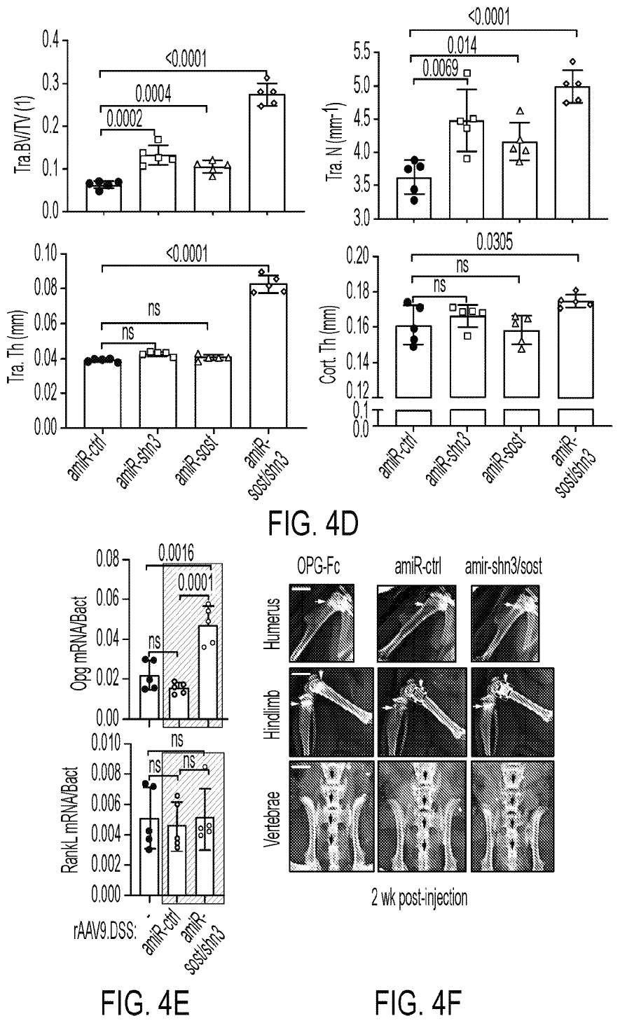

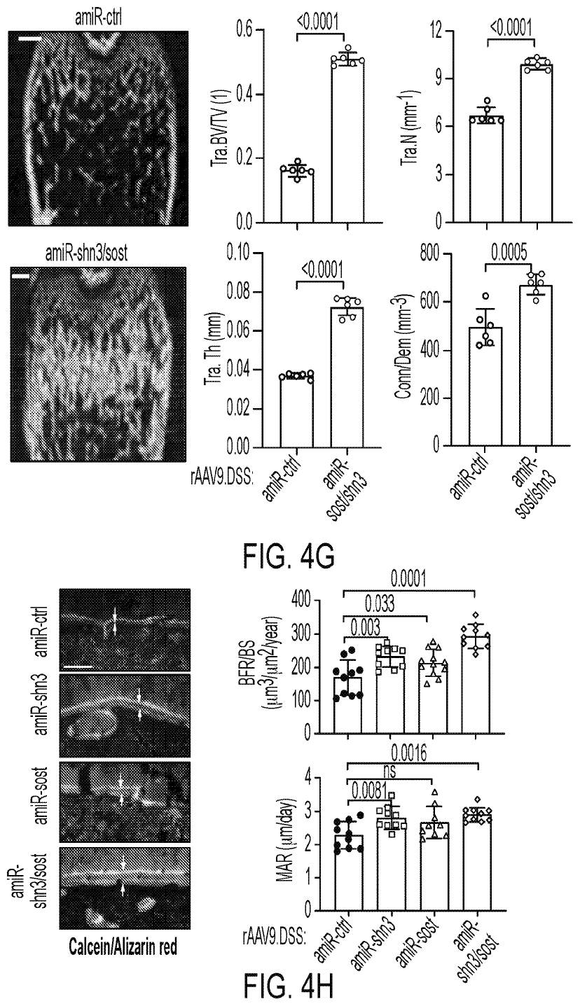

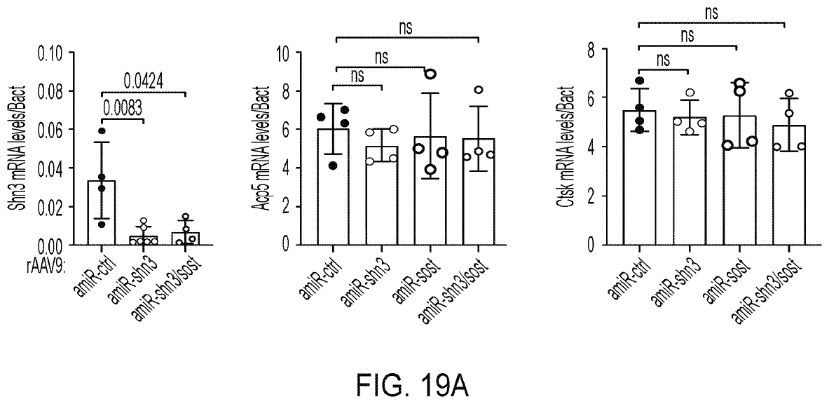

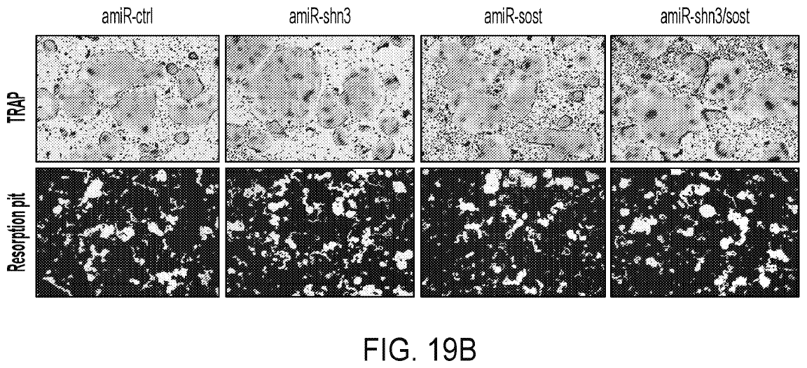

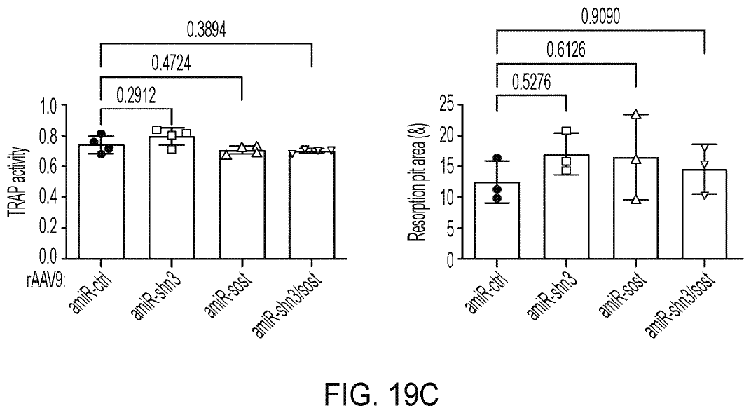

Since the WNT/β-catenin pathway in osteoblasts/osteocytes plays an inhibitory role in osteoclastogenesis via downregulation of receptor activator of nuclear factor kappa-B ligand (RANKL) and upregulation of the Rank decoy receptor osteoprotegerin (OPG), a single dose of AAV9.DSS.amiR-sost/shn3 was i.v. injected into one-month old mice, and RankL and OPG mRNA levels in the treated bones were assessed two weeks post-injection, demonstrating a significant increase in OPG expression without any alteration in RankL expression (FIG. 4E). Compared to amiR-ctrl-treated mice, these mice displayed a significant increase in trabecular bone mass in the epiphyseal area of long bones and vertebrae with high bone remodeling activity, whose effect is as potent as that of OPG-Fc fragment (FIGS. 4F, 17B). Similarly, trabecular bone mass in the dual silencer-treated femur was increased by ˜2.5 fold within two weeks (FIG. 4G). Since osteoclast differentiation and resorption activity were both normal in the dual silencer-treated bone marrow-derived monocytes (BMMs, FIG. 19), AAV9.DSS.amiR-sost/shn3 does not show any intrinsic effects on osteoclastogenesis. Thus, enhanced WNT/β-catenin signaling by AAV-mediated silencing of Shn3 and Sost is likely to upregulate OPG expression in osteoblasts/osteocytes, thereby suppressing osteoclast development in vivo.

In vivo osteoblast activity was increased in the trabecular bone in the metaphysis of these mice, as shown by greater bone formation rate (BFR) and mineral apposition rate (MAR) (FIG. 4H). Of note, the highest BFR and MAR were observed in the dual silencer-treated femurs. While the number of tartrate-resistant acid phosphatase (TRAP)-positive osteoclasts per bone surface and bone resorption activity per bone surface were unchanged in the femurs with single silencing of Sost or Shn3, dual silencing resulted in a significant decrease in osteoclast numbers and resorption activity (FIGS. 41 and 4J). This is accompanied with elevated levels of OPG in the femurs treated with a dual silencer, not a single silencer targeting Shn3 or Sost. These results demonstrate that systemically delivered AAV9.DSS.amiR-sost/shn3 enhanced WNT/β-catenin signaling in osteoblast lineage cells, augmented osteoblast activity and suppressed osteoclast development simultaneously, and increased bone mass. On the other hand, treatment with AAV9.DSS carrying amiR-shn3 or amiR-sost resulted in a mild increase in WNT/β-catenin signaling, osteoblast activity without any alteration in osteoclast number and function, and bone mass.

### Example 10. WNT-Modulating Gene Silencers Promote Bone Regeneration

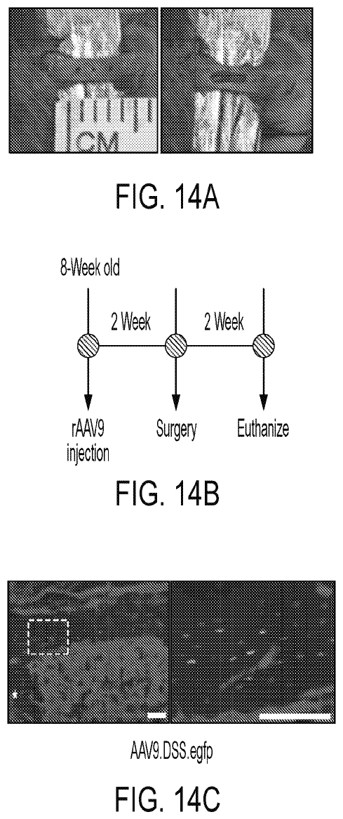

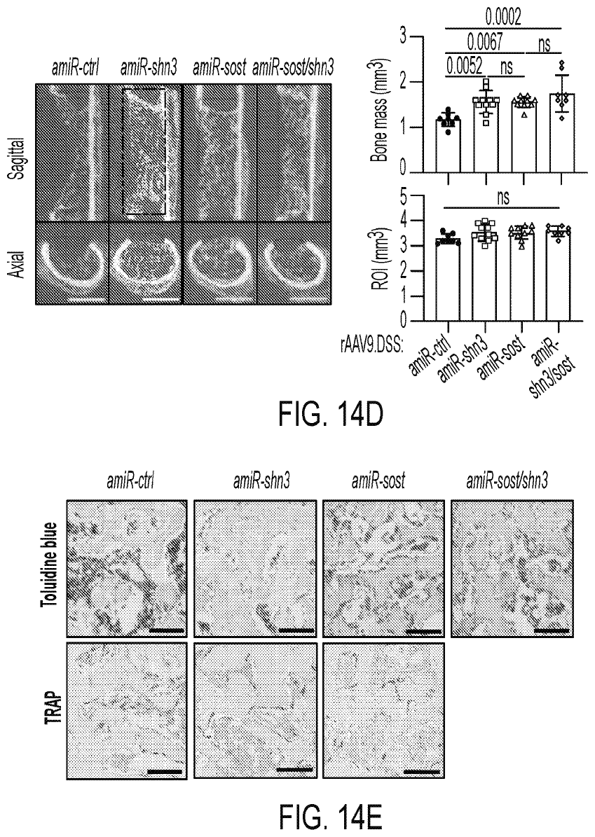

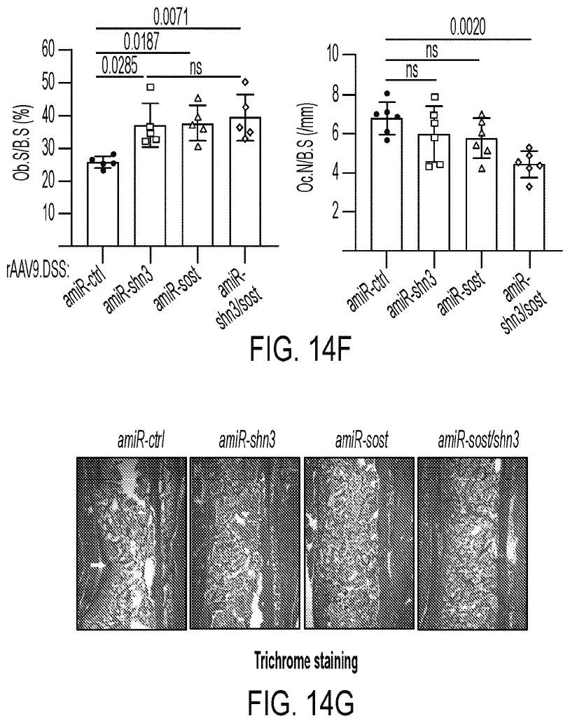

To examine the ability of our WNT-modulating gene silencers to promote bone regeneration, uni-cortical bone defect surgery was performed in the femur of 2-month-old mice This surgery was designed to eliminate the mechanical instability at the bone-injury site while preserving other cortical bones at the same level of the bone defect, which is a major factor determining the callus volume (FIG. 14A). Two weeks after i.v. injection of AAV9.DSS.egfp, GFP expression in the injured sites was monitored by fluorescence microscopy, validating the ability of the bone-specific AAV9 to express a transgene in the lesion of bone defect (FIG. 14B). Next, AAV9.DSS vectors carrying amiR-ctrl, amiR-shn3, amiR-sost, or amiR-sost/shn3 were i.v. injected 2 weeks prior to the surgery, and bone formation was assessed two weeks following the surgery (FIG. 14C). Reduced transcripts of Shn3 and/or Sost were validated in AAV-treated tibias (FIG. 14D). Bone formation was markedly increased in the bone defect areas when treated with a single or a dual silencer targeting Shn3 and/or Sost, as shown by increased volume of newly formed bones (FIG. 14E). This was accompanied with increased numbers of osteoblasts in the bone defect areas (FIGS. 14F and 14G). Notably, unlike a single silencer targeting Shn3 or Sost, amiR-sost/shn3-treated mice showed a significant decrease in osteoclast numbers (FIGS. 14F and 14G) while collagen formation in the bone defect areas was markedly increased (FIG. 14H). These results demonstrate that AAV-mediated silencing of Shn3 or/and Sost can promote osteoblast-mediated bone regeneration at early stage of bone healing process, and however, anti-resorptive effect occurs only when Shn3 and Sost are both silenced. Thus, the WNT-modulating gene silencers may be useful for the treatment of skeletal diseases with low bone mass as a potent bone anabolic agent.

### Example 11. WNT-Modulating Gene Silencers Promote Bone Fracture Repair

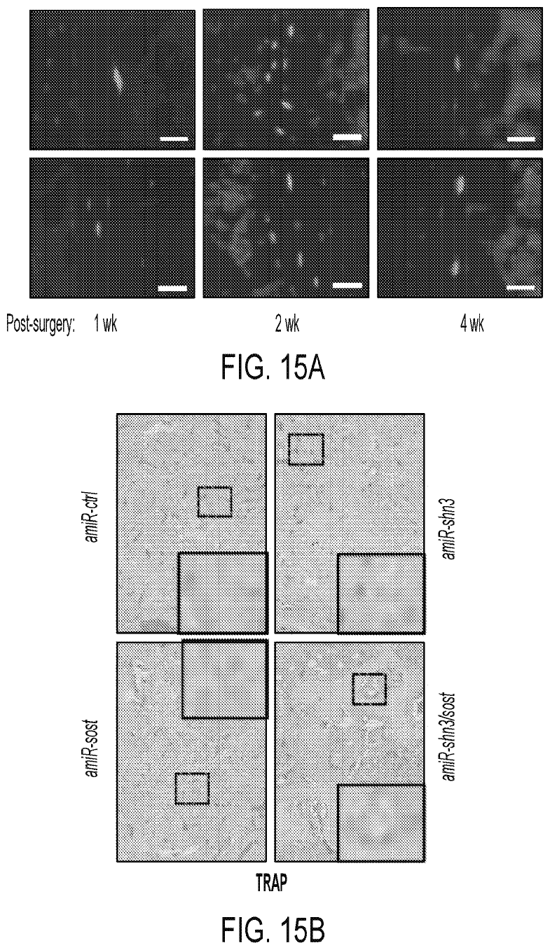

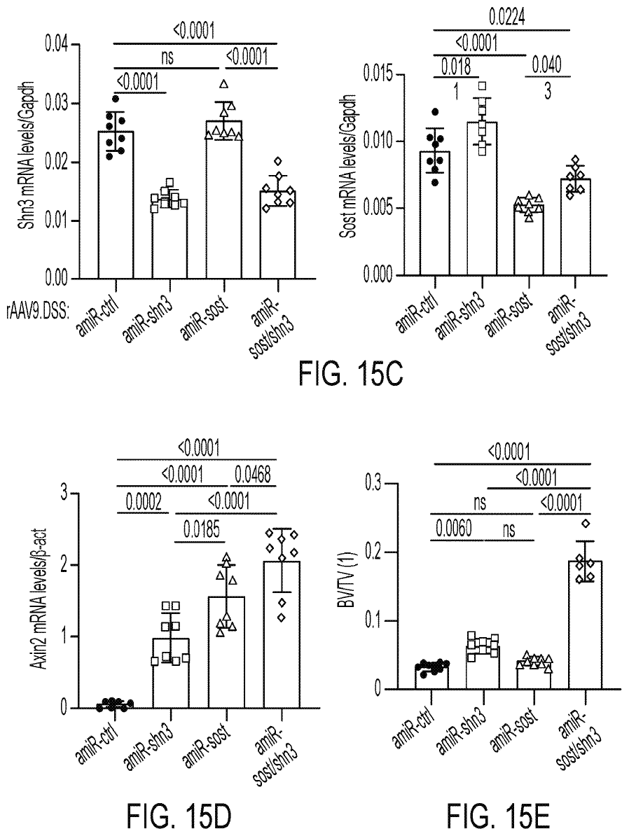

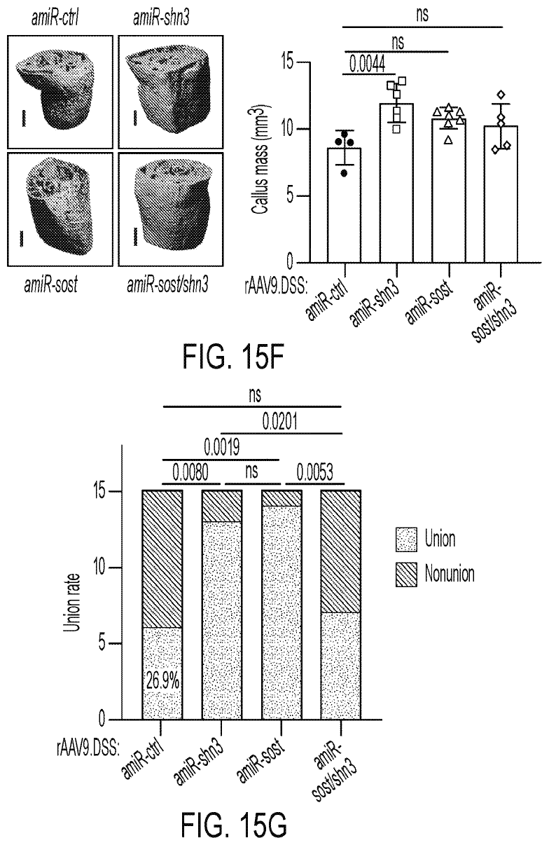

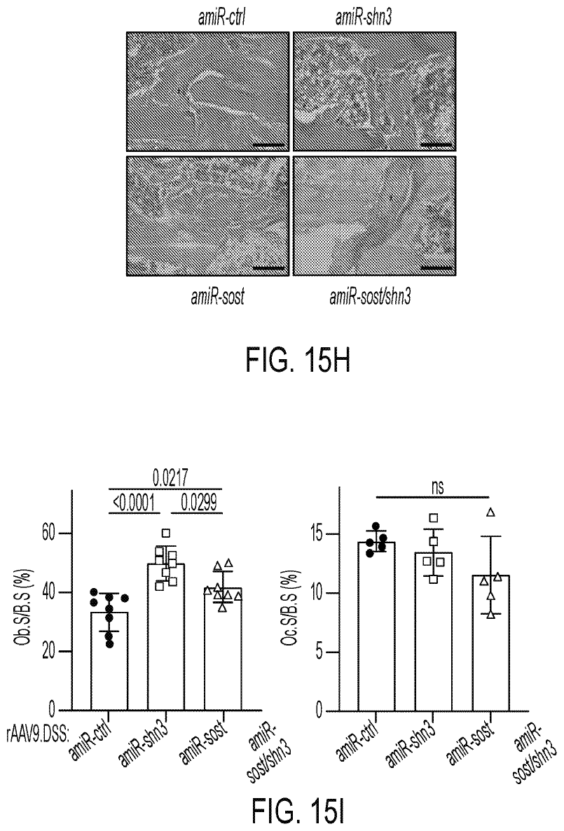

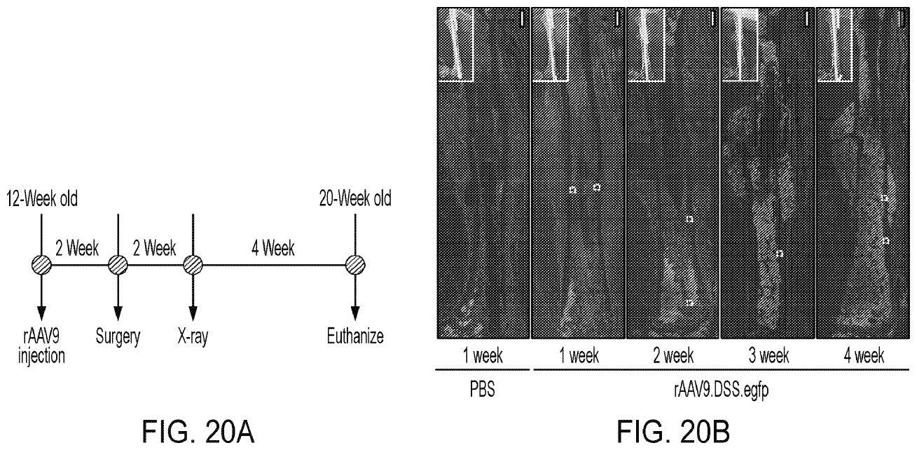

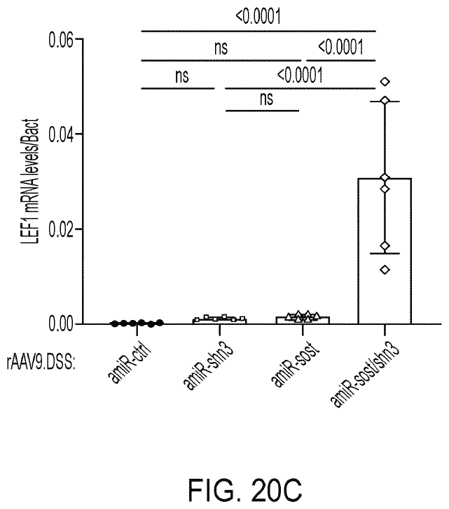

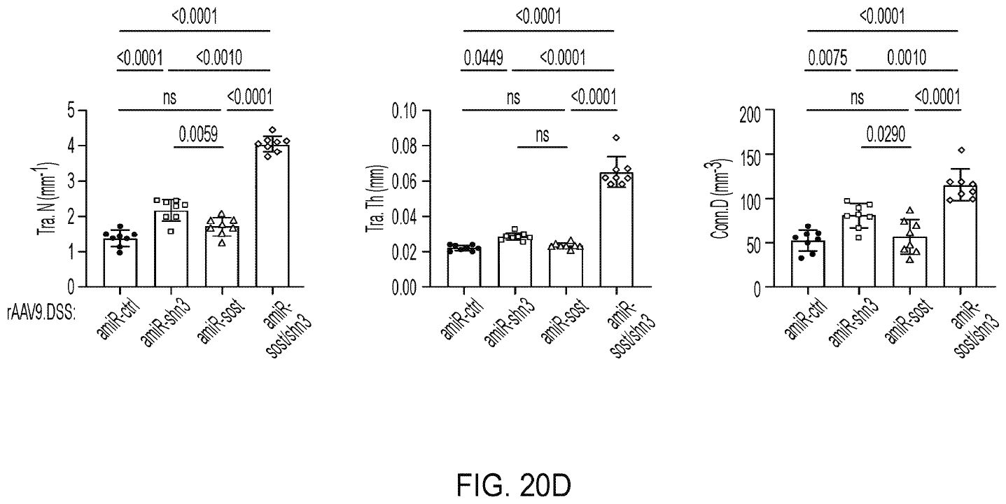

Bone regeneration and remodeling are important for bone formation at early process of fracture healing and for fracture unionization and restoration of biomechanical properties, respectively. Since the effects of anti-sclerostin antibody on bone fracture healing process are controversial, the effects of the WNT-modulating gene silencers disclosed herein on this process were examined using a mouse model of bone fracture. Two weeks after i.v. injection of AAV9.DSS.egfp, the fractured bones were fixed with an intramedullary semi-rigid following femoral osteotomy, and fracture healing was assessed using X-radiography 2 weeks post-fracture in live mice and microCT 6 weeks post-fracture after euthanasia. (FIGS. 15A, 20A). GFP expression in the fractured sites was monitored at different time points by fluorescence microscopy, validating the ability of the bone-specific AAV9 to transduce the cells residing in the lesion of bone fracture (FIGS. 15B, 20B). Next, AAV9.DSS vectors carrying amiR-ctrl, amiR-shn3, amiR-sost, or amiR-sost/shn3 were i.v. injected 2 weeks prior to the surgery, reduced transcripts of Shn3 and/or Sost were validated in AAV-treated tibias (FIG. 15C). In counterpart limbs with non-surgery, single silencing of Shn3 or Sost resulted a mild increase in the expression of Axin2 and Lef1 (FIGS. 15D, 20C) and trabecular bone mass (FIG. 15E, 20D), which was further upregulated when Shn3 and Sost were both silenced. These results confirmed the effects of our AAV vectors on WNT/β-catenin signaling and bone accrual. While early callus formation 2 weeks post-surgery was comparable in the fractured bones treated with amiR-shn3, amiR-sost, and amiR-sost/shn3, at 6 weeks post-surgery, the callus size of amiR-shn3-treated bone was bigger than that of amiR-sost- or amiR-sost/shn3-treated bones (FIG. 15F). These results suggest that unlike amiR-sost and amiR-sost/shn3, amiR-shn3 contributes to early callus formation. Compare to amiR-ctrl-treated femurs showing ˜26.9% fracture union rate 6 weeks post-surgery, fracture union rate was markedly increased by ??in amiR-shn3- or amiR-sost-treated femurs, respectively (FIG. 15G).

Likewise, histologic analysis of amiR-shn3- or amiR-sost-treated femurs showed enhanced connectivity between fracture ends by woven bone formation, whereas fibrosis occurred in the persistent nonunion sites of amiR-ctrl-treated femurs (FIG. 15H). This was accompanied with increased numbers of osteoblasts in the fractured sites of amiR-shn3- or amiR-sost-treated femurs while osteoclast numbers were relatively unchanged (FIG. 15I). Surprisingly, little to no improvement of fracture unionization was observed in amiR-sost/shn3-treated femurs (FIG. 15G) and similar to amiR-ctrl-treated femurs, fibrosis developed in persistent nonunion fracture sites of these femurs (FIG. 15H). Unlike amiR-shn3- or amiR-sost-treated femurs, osteoclast numbers were markedly decreased without any alteration in osteoblast numbers (FIG. 15I). Given the importance of bone remodeling in fracture unionization, reduced osteoclastogenesis by dual silencing slows down bone remodeling, hampering fracture unionization. On the other hand, single silencing of Shn3 or Sost promotes osteoblast-mediated bone formation without disturbing osteoclast-mediated bone resorption, improving fracture repair. It is speculated that WNT modulation by AAV gene silencers could affect early callus formation, but not late callus volume, which was unlikely to depend on the bone-forming activity of gene silencers but other factors such as the degree and duration of unstable condition at the fracture site.

TABLE 2
Sequences

| Gene | Forward | Reverse |
|---|---|---|
| Human Shn3 | GCCCTATGTGTGCAAGCACT | AGTCCTGGAACAGGTCGTCACT |
| | GT (SEQ ID NO: 19) | (SEQ ID NO: 28) |
| Mouse Shn3 | AGAGGCCATTCAGACGAGTG | CTGCGGAAGCTGAGAGATGT |
| | T (SEQ ID NO: 20) | (SEQ ID NO: 29) |
| Mouse Gapdh | ACTGAGCAAGAGAGGCCCTA | TATGGGGGTCTGGGATGGAA |
| | (SEQ ID NO: 21) | (SEQ ID NO: 30) |
| Mouse Actb | AGGGAAATCGTGCGTGACAT | GAACCGCTCGTTGCCAATAG |
| | (SEQ ID NO: 22) | (SEQ ID NO: 31) |
| Mouse Lef1 | CCAAGCAAGGCATGTCCAGA | GCCTGACAGTGAGGATGGGTAG |
| | CACC (SEQ ID NO: 23) | GG (SEQ ID NO: 32) |
| Mouse Axin2 | GCAGATGAACCTGAAGGATA | TTGATGCCATCTCGTATGTAGG |
| | CC (SEQ ID NO: 24) | (SEQ ID NO: 33) |
| Mouse Sost | CTTCAGGAATGATGCCACAG | ATCTTTGGCGTCATAGGGATGG |
| | AGGT (SEQ ID NO: 25) | TG (SEQ ID NO: 34) |
| Mouse Hprt | CTGGTGAAAAGGACCTCTCG | CCAGTTTCACTAATGACACAAA |
| | AAG (SEQ ID NO: 26) | CG (SEQ ID NO: 35) |
| EGFP | AGCAAAGACCCCAACGAGAA | GGCGGCGGTCACGAA (SEQ ID |
| | (SEQ ID NO: 27) | NO: 36) |

| | |
|---|---|
| EGFP-probe | 6FAM-CGCGATCACATGGTCCTGCTGG-TAMRA (SEQ ID NO: 37) |
| (AspSerSer)₆ | GATTCATCAGATTCTTCTGATTCATCCGACTCTTCTGACAGTTCA |
| | GACAGCTCT (SEQ ID NO: 1) |
| (Asp)₁₄ | GATGATGATGATGATGATGATGATGATGATGATGATGATGAT |
| | (SEQ ID NO: 2) |
| amiR-33-ctrl | tttgtcttttatttcaggtcccagatctagggctctgcgtttgctccaggtagtccgctgctcccttgggcctg |
| (amiR-ctrl) | ggcccactgacagccctggtgcctctggccggctgcacacctcctggcgggcagctgtgttactgtaggatcg |
| | agagggatgttctggcaatacctgtccctctccttactacagtaacacggaggcctgccctgactgcccacg |
| | gtgccgtggccaaagaggatctaagggcaccgctgagggcctacctaaccatcgtggggaataaggaca |
| | gtgtcacccctgcaggggatccggtggtggtgcaaatca (SEQ ID NO: 3) |
| Human amiR-33-mouse | gatctggcagccttggagtgggttcctgccccctcgggcacacaaacagagctgaagaccaccctgggca |
| shn3 (hs-amiR-shn3) | cctccttggctggccgcatacctcctggcgggcagctgtgtacaaactacttgagagcaggtgttctggtgg |
| | tacccacctgctctgtaatagtttgtacacagaggcctgcctggccctcgagagactgccctgactgaaggc |
| | cctatcaggtgggggaggggatcctgatagagggcactgctgccactgttggggcccaagctgca |
| | (SEQ ID NO: 4) |
| amiR-33-mouse | tttgtcttttatttcaggtcccagatctagggctctgcgtttgctccaggtagtccgctgctcccttgggcctg |
| shn3 (amiR-shn3) | ggcccactgacagccctggtgcctctggccggctgcacacctcctggcgggcagctgtgtacaaactacttga |
| | gagcaggtgttctggcaatacctgcctgctctgtaatagtttgtacacggaggcctgccctgactgcccacg |
| | gtgccgtggccaaagaggatctaagggcaccgctgagggcctacctaaccatcgtggggaataagg |
| | acagtgtcacccctgcaggggatccggtggtggtgcaaatca (SEQ ID NO: 5) |
| amiR-33-human | gtcttttatttcaggtcccagatcttagggctctgcgtttgctccaggtagtccgctgctcccttgggcctggg |
| shn3 (amiR-hshn3) | cccactgacagccctggtgcctctggccggctgcacacctcctggcgggcagctgtgtttccatggtaagttc |
| | aaggctgttctggcaatacctggccttgaagatgccatggaaacacggaggcctgccctgactgcccacg |
| | gtgccgtggccaaagaggatctaagggcaccgctgagggcctacctaaccatcgtggggaataaggaca |
| | gtgtcacccccctgcaggggatccggtggtggtgcaaat (SEQ ID NO: 6) |
| amiR-33-mouse | gatctagggctctgcgtttgctccaggtagtccgctgctcccttgggcctgggcccactgacagccctggtg |
| sost (amiR-sost) | cctctggccggctgcacacctcctggcgggcagctgtgtgacctctgtggcatcattcctgttctggcaatac |
| | ctgggaatgatcgcgcagaggtcacacggaggcctgccctgactgcccacggtgccgtggccaaagag |
| | gatctaagggcaccgctgagggcctacctaaccatcgtggggaataaggacagtgtcacccctgca |
| | (SEQ ID NO: 7) |
| amiR-33-mouse | gatctagggctctgcgtttgctccaggtagtccgctgctcccttgggcctgggcccactgacagccctggtg |
| sost (amiR-sost) | cctctggccggctgcacacctcctggcgggcagctgtgtgacctctgtggcatcattcctgttctggcaatac |
| and | ctgggaatgatcgcgcagaggtcacacggaggcctgccctgactgcccacggtgccgtggccaaagag |
| human amiR-33- | gatctaagggcaccgctgagggcctacctaaccatcgtggggaataaggacagtgtcacccctgcaggg |
| mouse shn3 | gatccggtggtggtgcaaatcaaagaactgctcctcagtggatgttgcctttacttctaggcctgtacggaag |
| (hs-amiR-shn3) | tgttacttctgctctaaaagctgcggaattgtacccgcggccgatccaccggtcgccaccatggggcagcct |
| | tggagtgggttcctgccccctcgggcacacaaacagagctgaagaccaccctgggcacctccttggctgg |
| | ccgcatacctcctggcgggcagctgtgtacaaactacttgagagcaggtgttctggtggtacccacctgctc |
| | tgtaatagtttgtacacagaggcctgcctggccctcgagagactgccctgactgaaggccctatcaggtgg |
| | gggaggggatcctgatagagggcactgctgccactgttggggcccaagaagct (SEQ ID NO: 8) |
| Human amiR-33-human | ggcagccttggagtgggttcctgccccctcgggcacacaaacagagctgaagaccaccctgggcacctc |
| shn3-1 | cttggctggccgcatacctcctggcgggcagctgtgtttccatggtaagttcaaggctgttctggtggtaccc |
| (hs-amiR-hshn3-1) | agccttgaagatgccatggaaacacagaggcctgcctggccctcgagagactgccctgactgaaggccct |
| | atcaggtgggggaggggatcctgatagagggcactgctgccactgttggggcccaag (SEQ ID |
| | NO: 9) |
| Human amiR-33-human | ggcagccttggagtgggttcctgccccctcgggcacacaaacagagctgaagaccaccctgggcacctc |
| shn3-2 | cttggctggccgcatacctcctggcgggcagctgtgtccatggtaagttcaaggctgtgttctggtggtaccc |
| (hs-amiR-hshn3-2) | acagccttgttcctaccatggacacagaggcctgcctggccctcgagagactgccctgactgaaggcccta |
| | tcaggtgggggaggggatcctgatagagggcactgctgccactgttggggcccaag (SEQ ID NO: |
| | 10) |
| DSS.AAV9 | DSSDSSDSSDSSDSSDSS (SEQ ID NO: 11) |

### Exemplary Embodiments

The following are some non-limiting embodiments of this disclosure:
1. A bone graft substitute comprising a recombinant adeno-associated virus (rAAV) and hydroxyapatite (HA) attached to the bone graft substitute, wherein the rAAV comprises a capsid protein comprising a peptide motif and an isolated nucleic acid comprising a nucleic acid sequence encoding an inhibitory nucleic acid targeting sclerostin (SOST), schnurri-3 (SHN3), or SOTS and SHN3, wherein the bone graft substitute is for the implantation to a subject.
2. The bone graft substitute of embodiment 1, wherein the peptide motif comprises the amino acid sequence DSSDSSDSSDSSDSSDSS (SEQ ID NO: 11).
3. The bone graft substitute of embodiment 1 or 2, wherein the bone graft substitute is an allogeneic bone graft.
4. The bone graft substitute of any one of embodiments 1-3, wherein the capsid protein is an AAV9 capsid protein.
5. The bone graft substitute of any one of embodiments 1-4, wherein the bone graft substitute is incubated ex vivo with the rAAV prior to implantation to the subject.
6. The bone graft substitute of any one of embodiments 1-4, wherein the bone graft substitute is incubated ex vivo with human bone marrow-derived stromal cells prior to implantation to the subject.
7. The bone graft substitute of any one of embodiments 1-4, wherein the bone graft substitute is incubated ex vivo with a composition comprising cells of osteoblastic lineage prior to implantation to the subject.
8. The bone graft substitute of any one of embodiments 1-7, wherein the inhibitory nucleic acid is an ami-RNA comprising a human miRNA backbone, optionally a human miR-33 backbone.
9. The bone graft substitute of any one of embodiments 1-7, wherein the inhibitory nucleic acid is an ami-RNA comprising a mouse miRNA backbone, optionally a mouse miR-33 backbone.
10. The bone graft substitute of any one of embodiments 1-9, wherein the inhibitory nucleic acid targets SOST, optionally wherein the inhibitory nucleic acid is encoded by a nucleic acid comprising the sequence set forth in SEQ ID NO: 7.
11. The bone graft substitute of any one of embodiments 1-9, wherein the inhibitory nucleic acid targets SHN3 and SOST, optionally wherein the inhibitory nucleic acid is encoded by a nucleic acid comprising the sequence set forth in SEQ ID NO: 8
12. The bone graft substitute of any one of embodiments 1-11, wherein the subject is a human.
13. An isolated nucleic acid comprising a transgene comprising a chicken D-actin (CB) promoter operably linked to a nucleic acid sequence encoding an inhibitory nucleic acid targeting sclerostin (SOST), schnurri-3 (SHN3), or SOST and SHN3. 14. The isolated nucleic acid of embodiment 13, wherein the transgene encodes an inhibitory nucleic acid selected from the group consisting of dsRNA, siRNA, shRNA, miRNA, and artificial miRNA (amiRNA).
15. The isolated nucleic acid of embodiment 13 or 14, wherein the inhibitory nucleic acid is an ami-RNA comprising a human miRNA backbone, optionally a human miR-33 backbone.
16. The isolated nucleic acid of embodiment 12 or 14, wherein the inhibitory nucleic acid is an ami-RNA comprising a mouse miRNA backbone, optionally a mouse miR-33 backbone.
17. The isolated nucleic acid of any one of embodiments 13 to 16, wherein the inhibitory nucleic acid targets SHN3, optionally wherein the inhibitory nucleic acid is encoded by a nucleic acid comprising the sequence set forth in any one of SEQ ID NOs: 4, 5, 6, 9 and 10.
18. The isolated nucleic acid of any one of embodiments 13 to 16, wherein the inhibitory nucleic acid targets SOST, optionally wherein the inhibitory nucleic acid is encoded by a nucleic acid comprising the sequence set forth in SEQ ID NO: 7.
19. The isolated nucleic acid of any one of embodiments 13 to 16, wherein the inhibitory nucleic acid targets SHN3 and SOST, optionally wherein the inhibitory nucleic acid is encoded by a nucleic acid comprising the sequence set forth in SEQ ID NO: 8.
20. The isolated nucleic acid of any one of embodiments 13-9, wherein the transgene further comprises a CMV enhancer sequence.
21. The isolated nucleic acid of any one of embodiments 13 to 20, wherein the transgene is flanked by adeno-associated virus (AAV) inverted terminal repeats (ITRs).
22. The isolated nucleic acid of embodiment 21, wherein the AAV ITRs are AAV2 ITRs.
23. An isolated nucleic acid comprising or encoding a sequence set forth in any one of SEQ ID NOs: 1-11.
24. A vector comprising the isolated nucleic acid of any one of embodiments 1 to 23.
25. The vector of embodiment 24, wherein the vector is a plasmid, bacmid, cosmid, viral, closed-ended linear DNA (ceDNA), or Baculovirus vector.
26. The vector of embodiment 24 or 25, wherein the vector is a recombinant adeno-associated virus (rAAV) vector, retroviral vector, or adenoviral vector.
27. A recombinant adeno-associated virus (rAAV) comprising:
   (i) the isolated nucleic acid of any one of embodiments 1 to 11; and
   (ii) at least one AAV capsid protein.
28. The rAAV of embodiment 27, wherein the AAV capsid protein is of a serotype selected from AAV1, AAV2, AAV3, AAV4, AAV5, AAV6, AAV6.2, AAV7, AAV8, AAV9, AAV.rh8, AAV.rh10, AAV.rh39, AAV.rh43, AAV2/2-66, AAV2/2-84, and AAV2/2-125, or a variant of any of the foregoing.
29. The rAAV of any one of embodiments 27 to 28, wherein the AAV capsid protein comprises the amino acid sequence DSSDSSDSSDSSDSSDSS (SEQ ID NO: 11).
30. The rAAV of any one of embodiments 27 to 29, further comprising attaching to a hydroxyapatite (HA) scaffold.
31. The rAAV of embodiment 30, wherein the attachment to the HA scaffold improves the bone-specific tropism of the rAAV, optionally wherein the rAAV is rAAV9.
32. A composition comprising the rAAV of any one of embodiments 27 to 31, and a pharmaceutically acceptable excipient.
33. A method for delivering a transgene to a bone tissue in a subject, the method comprising administering to a subject the isolated nucleic acid of any one of embodiments 13 to 23, the vector of any one of embodiments 24-26, the rAAV of any one of embodiments 27 to 31, or the bone graft substitute of embodiments 1-12.
34. A method for treating a disease or disorder associated with bone fracture and critical-sized bone defect in a subject, the method comprising administering to a subject the isolated nucleic acid of any one of embodiments 13 to 23, the vector of any one of embodiments 24-26, the rAAV of any one of embodiments 27 to 31, or the bone graft substitute of embodiments 1-12.
35. A method for treating a disease or disorder associated with osteoporosis in a subject, the method comprising administering to a subject the isolated nucleic acid of any one of embodiments 13 to 23, the vector of any one of embodiments 24 to 26, the rAAV of any one of embodiments 27 to 31, or the bone graft substitute of embodiments 1-12, optionally wherein the osteoporosis is senile osteoporosis.
36. A method for improving bone formation and/or bone healing in a subject, the method comprising administering to a subject the isolated nucleic acid of any one of embodiments 13 to 23, the vector of any one of embodiments 24-26, the rAAV of any one of embodiments 27 to 31, or the bone graft substitute of embodiments 1-12.
37. A method for stimulating bone regeneration and/or reversing bone loss in a subject, the method comprising administering to a subject the isolated nucleic acid of any one of embodiments 13 to 23, the vector of any one of embodiments 24-26, the rAAV of any one of embodiments 27 to 31, or the bone graft substitute of embodiments 1-12.
38. The method of any one of embodiments 33 to 37, wherein the administration occurs by injection, optionally wherein the injection is systemic injection or local injection.
39. The method of embodiment 38, wherein the systemic injection comprises intravenous 10 injection.
40. The method of embodiment 38, wherein the local injection comprises intramuscular (IM) injection, knee injection, or femoral intramedullary injection.
41. The method of any one of embodiments 33-40, wherein the administration results in an increase of receptor activator of nuclear factor kappa-B ligand (RANKL), osteoprotegerin (OPG), Axin2 and/or Lef1 compared to a control.
42. The method of any one of embodiments 33-41, wherein the subject is a human.

What is claimed is:

1. A bone graft substitute comprising a recombinant adeno-associated virus (rAAV) and hydroxyapatite (HA) attached to the bone graft substitute, wherein the rAAV comprises a capsid protein comprising a peptide motif and an isolated nucleic acid comprising a nucleic acid sequence encoding an inhibitory nucleic acid targeting sclerostin (SOST), schnurri-3 (SHN3), or SOTS and SHN3, wherein the bone graft substitute is for the implantation to a subject.

2. The bone graft substitute of claim 1, wherein the peptide motif comprises the amino acid sequence DSSDSSDSSDSSDSSDSS (SEQ ID NO: 11).

3. The bone graft substitute of claim 1, wherein the bone graft substitute is an allogeneic bone graft.

4. The bone graft substitute of claim 1, wherein the capsid protein is an AAV9 capsid protein.

5. The bone graft substitute of claim 1, wherein the bone graft substitute is incubated ex vivo with the rAAV prior to implantation to the subject.

6. The bone graft substitute of claim 1, wherein the bone graft substitute is incubated ex vivo with human bone marrow-derived stromal cells prior to implantation to the subject.

7. The bone graft substitute of claim 1, wherein the inhibitory nucleic acid is an ami-RNA comprising a human miRNA backbone, optionally a human miR-33 backbone.

8. An isolated nucleic acid comprising a transgene comprising a chicken β-actin (CB) promoter operably linked to a nucleic acid sequence encoding an inhibitory nucleic acid targeting sclerostin (SOST), schnurri-3 (SHN3), or SOST and SHN3.

9. The isolated nucleic acid of claim 8, wherein the transgene encodes an inhibitory nucleic acid selected from the group consisting of dsRNA, siRNA, shRNA, miRNA, and artificial miRNA (amiRNA).

10. The isolated nucleic acid of claim 8, wherein the inhibitory nucleic acid is an ami-RNA comprising a human miRNA backbone, optionally a human miR-33 backbone.

11. The isolated nucleic acid of claim 9, wherein the inhibitory nucleic acid is an ami-RNA comprising a mouse miRNA backbone, optionally a mouse miR-33 backbone.

12. The isolated nucleic acid of claim 8, wherein the inhibitory nucleic acid targets SHN3, optionally wherein the inhibitory nucleic acid is encoded by a nucleic acid comprising the sequence set forth in any one of SEQ ID NOs: 4, 5, 6, 9 and 10.

13. The isolated nucleic acid of claim 8, wherein the inhibitory nucleic acid targets SOST, optionally wherein the inhibitory nucleic acid is encoded by a nucleic acid comprising the sequence set forth in SEQ ID NO: 7.

14. The isolated nucleic acid of claim 8, wherein the transgene further comprises a CMV enhancer sequence.

15. The isolated nucleic acid of claim 8, wherein the transgene is flanked by adeno-associated virus (AAV) inverted terminal repeats (ITRs).

16. The isolated nucleic acid of claim 15, wherein the AAV ITRs are AAV2 ITRs.

17. An isolated nucleic acid comprising or encoding a sequence set forth in any one of SEQ ID NOs: 1-11.

18. A vector comprising the isolated nucleic acid of claim 17.

19. The vector of claim 18, wherein the vector is a plasmid, bacmid, cosmid, viral, closed-ended linear DNA (ceDNA), or Baculovirus vector.

20. The vector of claim 18, wherein the vector is a recombinant adeno-associated virus (rAAV) vector, retroviral vector, or adenoviral vector.
